SEQUENCE LISTING (61 sequences)

SEQ ID NO: 1 - protein, 398 aa, Homo sapiens
  REGION 1..398: note = misc_feature - GAL matureprotein sequence (32-429 aa of NP_000160)
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - DNA, 1197 bp, Homo sapiens
  misc_feature 1..1197: note = GAL mature peptide nucleic acid sequence
  source 1..1197: mol_type = genomic DNA
ctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 3 - DNA, 1197 bp, synthetic construct
  misc_feature 1..1197: note = Synthetic: GAL mature peptide codon optimized nucleic acid sequenceversion A
  source 1..1197: mol_type = other DNA
ctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctatgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtga

SEQ ID NO: 4 - DNA, 1197 bp, synthetic construct
  misc_feature 1..1197: note = Synthetic: GAL mature peptide codon optimized nucleic acid sequenceversion B
  source 1..1197: mol_type = other DNA
ctcgataatgggctcgctcggactccaaccatgggctggctccactgggaacgcttcatgtgtaatctggactgccaagaagaaccagatagctgtatcagcgaaaaactgtttatggaaatggctgagctcatggtcagcgagggctggaaggacgccggctacgagtatctgtgtatcgacgactgctggatggcccctcagcgggattcagagggcaggctccaggctgacccacagcggtttccccacggaattcggcagctggccaattacgtgcacagcaaaggcctgaagctggggatctacgctgatgtggggaacaagacctgcgctggatttccaggcagcttcggctactacgatatcgacgcacagaccttcgccgattggggcgtcgacctcctgaaatttgatggatgctattgcgatagcctcgaaaacctggccgacgggtacaaacacatgagcctcgctctgaaccggaccggccggagcatcgtgtacagctgcgaatggcctctgtacatgtggcccttccagaagccaaattacacagagattagacagtattgcaatcattggcggaacttcgccgacatcgatgacagctggaagagcatcaagagcatcctcgactggacctcattcaatcaggaacgcattgtggacgtggcaggaccaggcgggtggaatgatcccgacatgctggtgattggcaactttggactgagctggaatcagcaggtcacccagatggcactgtgggctatcatggccgcccccctgtttatgagcaacgacctcaggcacatctcaccacaagccaaggcactgctccaggataaggatgtcattgcaattaatcaggatccactgggcaaacaaggctatcagctgagacagggagataatttcgaagtgtgggagcggcctctgagcggactggcatgggccgtggctatgatcaacagacaggagatcggcggcccaagatcatacacaatcgccgtggcatcactgggcaagggagtcgcctgcaaccccgcatgctttatcactcagctgctgcctgtgaaaaggaagctgggcttctacgaatggacaagcagactgaggagccacatcaaccccacaggaacagtcctgctccaactggaaaatactatgcaaatgtcactgaaagatctgctgtga

SEQ ID NO: 5 - DNA, 1197 bp, synthetic construct
  misc_feature 1..1197: note = Synthetic: GAL mature peptide codon optimized nucleic acid sequenceversion B CpG reduced
  source 1..1197: mol_type = other DNA
ctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtga

SEQ ID NO: 6 - protein, 31 aa, Homo sapiens
  REGION 1..31: note = misc_feature - GAL native signal peptide
MQLRNPELHLGCALALRFLALVSWDIPGARA

SEQ ID NO: 7 - DNA, 93 bp, Homo sapiens
  misc_feature 1..93: note = GAL native signal peptide coding sequence
  source 1..93: mol_type = genomic DNA
atgcagctgaggaacccagaactacatctgggctgcgcgcttgcgcttcgcttcctggccctcgtttcctgggacatccctggggctagagca

SEQ ID NO: 8 - DNA, 93 bp, synthetic construct
  misc_feature 1..93: note = Synthetic: GAL native signal peptide codon optimized version A
  source 1..93: mol_type = other DNA
atgcagctgagaaatcctgagctgcacctgggctgtgccctggctctgagatttctggctctggtgtcctgggacatccctggcgctagagcc

SEQ ID NO: 9 - DNA, 93 bp, synthetic construct
  misc_feature 1..93: note = Synthetic: GAL native signal peptide codon optimized version B
  source 1..93: mol_type = other DNA
atgcagctcagaaacccagaactccatctggggtgcgctctcgccctccgcttcctggcactcgtcagctgggatattcctggggctagagcc

SEQ ID NO: 10 - DNA, 93 bp, synthetic construct
  misc_feature 1..93: note = Synthetic: GAL native signal peptide codon optimized version B CpG reduced
  source 1..93: mol_type = other DNA
atgcagctcagaaacccagaactccatctggggtgtgccctggccctgaggttcctggcactggtgagctgggatattcctggggctagagcc

SEQ ID NO: 11 - protein, 19 aa, synthetic construct
  REGION 1..19: note = Synthetic: Human and mouse IgG1 signal peptideMIgG1 (Genbank ID: AAA51634) and hIgG1 (GenBank ID: QBK47409 heavy chain
MGWSCIILFLVATATGVHS

SEQ ID NO: 12 - DNA, 57 bp, Homo sapiens
  misc_feature 1..57: note = Human IgG1 reverse translated coding sequence
  source 1..57: mol_type = genomic DNA
atgggctggagctgcatcatcctgttcctggtggccaccgccaccggcgtgcacagc

SEQ ID NO: 13 - DNA, 57 bp, synthetic construct
  misc_feature 1..57: note = Synthetic: Human IgG1 codon optimized C
  source 1..57: mol_type = other DNA
atggggtggtcatgtattatcctgtttctggtcgctaccgcaaccggagtgcacagc

SEQ ID NO: 14 - DNA, 57 bp, synthetic construct
  misc_feature 1..57: note = Synthetic: Human IgG1 codon optimized C CpG reduced
  source 1..57: mol_type = other DNA
atggggtggtcatgtattatcctgtttctggtggccacagccacaggcgtgcacagc

SEQ ID NO: 15 - protein, 18 aa, synthetic construct
  REGION 1..18: note = Synthetic: Synthetic IgG1 signal peptide
MGWSCILFLVATATGVHS

SEQ ID NO: 16 - DNA, 54 bp, synthetic construct
  misc_feature 1..54: note = Synthetic: Synthetic IgG1 reverse transcribed
  source 1..54: mol_type = other DNA
atgggctggagctgcattctgtttctggtggcgaccgcgaccggcgtgcatagc

SEQ ID NO: 17 - DNA, 145 bp, synthetic construct
  misc_feature 1..145: note = Synthetic: 5 ITR
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 18 - DNA, 145 bp, synthetic construct
  misc_feature 1..145: note = Synthetic: 3 ITR
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 19 - DNA, 321 bp, synthetic construct
  misc_feature 1..321: note = Synthetic: Apo E/C-I
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 20 - DNA, 393 bp, synthetic construct
  misc_feature 1..393: note = Synthetic: A1AT promoter
  source 1..393: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 21 - DNA, 476 bp, synthetic construct
  misc_feature 1..476: note = Synthetic: b-globin intron
  source 1..476: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacag

SEQ ID NO: 22 - DNA, 12 bp, synthetic construct
  misc_feature 1..12: note = Synthetic: Kozak sequence
  source 1..12: mol_type = other DNA
gccgccaccatg

SEQ ID NO: 23 - DNA, 225 bp, synthetic construct
  misc_feature 1..225: note = Synthetic: Bovine growth hormone poly A
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 24 - DNA, 134 bp, synthetic construct
  misc_feature 1..134: note = Synthetic: SV40 polyA
  source 1..134: mol_type = other DNA
gctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 25 - DNA, 590 bp, synthetic construct
  misc_feature 1..590: note = Synthetic: WPRE 590bp
  source 1..590: mol_type = other DNA
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 26 - DNA, 249 bp, synthetic construct
  misc_feature 1..249: note = Synthetic: W3SL 249bp
  source 1..249: mol_type = other DNA
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 27 - protein, 429 aa, synthetic construct
  REGION 1..429: note = Synthetic: GAL with native signal
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 28 - protein, 417 aa, synthetic construct
  REGION 1..417: note = Synthetic: GAL with IgG1 signal
MGWSCIILFLVATATGVHSLDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 29 - protein, 429 aa, Homo sapiens; identical to SEQ ID NO: 27
  REGION 1..429: note = misc_feature - GAL protein- > NP_000160.1 alpha-galactosidase A precursor

SEQ ID NO: 30 - DNA, 1318 bp, Homo sapiens
  misc_feature 1..1318: note = GLA - NM_000169.3 Homo sapiens galactosidase alpha (GLA),transcript variant 1, mRNA
  source 1..1318: mol_type = genomic DNA
atgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaaatgtt

SEQ ID NO: 31 - DNA, 3246 bp, synthetic construct
  misc_feature 1..3246: note = Synthetic: Construct 1A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), hGLA signal peptide (SEQ ID 7), mature peptide (SEQ ID 2), bGH polyA (SEQ ID NO 23), 3ITR (SEQ ID NO: 18)
  source 1..3246: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 32 - DNA, 3246 bp, synthetic construct
  misc_feature 1..3246: note = Synthetic: Construct 2A- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), GLA coA signal peptide (SEQ ID 8), coA mature peptide (SEQ ID 3), bGH polyA (SEQ ID NO 23), 3ITR (SEQ ID NO: 18)
  source 1..3246: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctgagaaatcctgagctgcacctgggctgtgccctggctctgagatttctggctctggtgtcctgggacatccctggcgctagagccctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctatgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 33 - DNA, 3246 bp, synthetic construct
  misc_feature 1..3246: note = Synthetic: Construct 3A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), GLA coB signal peptide (SEQ ID 9), coB mature peptide (SEQ ID 4), bGH polyA (SEQ ID NO 23), 3ITR (SEQ ID NO: 18)
  source 1..3246: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctcagaaacccagaactccatctggggtgcgctctcgccctccgcttcctggcactcgtcagctgggatattcctggggctagagccctcgataatgggctcgctcggactccaaccatgggctggctccactgggaacgcttcatgtgtaatctggactgccaagaagaaccagatagctgtatcagcgaaaaactgtttatggaaatggctgagctcatggtcagcgagggctggaaggacgccggctacgagtatctgtgtatcgacgactgctggatggcccctcagcgggattcagagggcaggctccaggctgacccacagcggtttccccacggaattcggcagctggccaattacgtgcacagcaaaggcctgaagctggggatctacgctgatgtggggaacaagacctgcgctggatttccaggcagcttcggctactacgatatcgacgcacagaccttcgccgattggggcgtcgacctcctgaaatttgatggatgctattgcgatagcctcgaaaacctggccgacgggtacaaacacatgagcctcgctctgaaccggaccggccggagcatcgtgtacagctgcgaatggcctctgtacatgtggcccttccagaagccaaattacacagagattagacagtattgcaatcattggcggaacttcgccgacatcgatgacagctggaagagcatcaagagcatcctcgactggacctcattcaatcaggaacgcattgtggacgtggcaggaccaggcgggtggaatgatcccgacatgctggtgattggcaactttggactgagctggaatcagcaggtcacccagatggcactgtgggctatcatggccgcccccctgtttatgagcaacgacctcaggcacatctcaccacaagccaaggcactgctccaggataaggatgtcattgcaattaatcaggatccactgggcaaacaaggctatcagctgagacagggagataatttcgaagtgtgggagcggcctctgagcggactggcatgggccgtggctatgatcaacagacaggagatcggcggcccaagatcatacacaatcgccgtggcatcactgggcaagggagtcgcctgcaaccccgcatgctttatcactcagctgctgcctgtgaaaaggaagctgggcttctacgaatggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 34 - DNA, 3246 bp, synthetic construct
  misc_feature 1..3246: note = Synthetic: Construct 4A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), GLA coB-CpG signal peptide (SEQ ID 10), coB-CpG mature peptide (SEQ ID 5), bGH polyA (SEQ ID NO 23), 3ITR (SEQ ID NO: 18)
  source 1..3246: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctcagaaacccagaactccatctggggtgtgccctggccctgaggttcctggcactggtgagctgggatattcctggggctagagccctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 35 - DNA, 3210 bp, synthetic construct
  misc_feature 1..3210: note = Synthetic: Construct 5A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 signal peptide (SEQ ID 11 and 12), mature peptide (SEQ ID 2), bGH polyA (SEQ ID NO 23), 3ITR (SEQ ID NO: 18)
  source 1..3210: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgggctggagctgcatcatcctgttcctggtggccaccgccaccggcgtgcacagcctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 36 - DNA, 3210 bp, synthetic construct
  misc_feature 1..3210: note = Synthetic: Construct 6A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC signal peptide (SEQ ID 13), coA mature peptide (SEQ ID 3), bGH polyA (SEQ ID NO 23), 3ITR (SEQ ID NO: 18)
  source 1..3210: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtcgctaccgcaaccggagtgcacagcctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctatgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 37 - DNA, 3210 bp, synthetic construct
  misc_feature 1..3210: note = Synthetic: Construct 7A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC signal peptide (SEQ ID 13), coB mature peptide (SEQ ID 4), bGH polyA (SEQ ID NO 23), 3ITR (SEQ ID NO: 18)
  source 1..3210: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtcgctaccgcaaccggagtgcacagcctcgataatgggctcgctcggactccaaccatgggctggctccactgggaacgcttcatgtgtaatctggactgccaagaagaaccagatagctgtatcagcgaaaaactgtttatggaaatggctgagctcatggtcagcgagggctggaaggacgccggctacgagtatctgtgtatcgacgactgctggatggcccctcagcgggattcagagggcaggctccaggctgacccacagcggtttccccacggaattcggcagctggccaattacgtgcacagcaaaggcctgaagctggggatctacgctgatgtggggaacaagacctgcgctggatttccaggcagcttcggctactacgatatcgacgcacagaccttcgccgattggggcgtcgacctcctgaaatttgatggatgctattgcgatagcctcgaaaacctggccgacgggtacaaacacatgagcctcgctctgaaccggaccggccggagcatcgtgtacagctgcgaatggcctctgtacatgtggcccttccagaagccaaattacacagagattagacagtattgcaatcattggcggaacttcgccgacatcgatgacagctggaagagcatcaagagcatcctcgactggacctcattcaatcaggaacgcattgtggacgtggcaggaccaggcgggtggaatgatcccgacatgctggtgattggcaactttggactgagctggaatcagcaggtcacccagatggcactgtgggctatcatggccgcccccctgtttatgagcaacgacctcaggcacatctcaccacaagccaaggcactgctccaggataaggatgtcattgcaattaatcaggatccactgggcaaacaaggctatcagctgagacagggagataatttcgaagtgtgggagcggcctctgagcggactggcatgggccgtggctatgatcaacagacaggagatcggcggcccaagatcatacacaatcgccgtggcatcactgggcaagggagtcgcctgcaaccccgcatgctttatcactcagctgctgcctgtgaaaaggaagctgggcttctacgaatggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 38 - DNA, 3210 bp, synthetic construct
  misc_feature 1..3210: note = Synthetic: Construct 8A- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), bGH polyA (SEQ ID NO 23), 3ITR (SEQ ID NO: 18)
  source 1..3210: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 39 - DNA, 3827 bp, synthetic construct
  misc_feature 1..3827: note = Synthetic: Construct 9A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), WPRE (SEQ ID NO 25),bGH polyA (SEQ ID NO 23), 3ITR (SEQ ID NO: 18)
  source 1..3827: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctatttgtgaaatttgtgatgctattgctttatttgtaaccatctactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 40 - DNA, 3733 bp, synthetic construct
  misc_feature 1..3733: note = Synthetic: Construct 10A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), WPRE (SEQ ID NO 25),SV40 polyA (SEQ ID NO 24), 3ITR (SEQ ID NO: 18)
  source 1..3733: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 41 - DNA, 3395 bp, synthetic construct
  misc_feature 1..3395: note = Synthetic: Construct 11A - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), W3SL (SEQ ID NO 26),SV40 polyA (SEQ ID NO 24), 3ITR (SEQ ID NO: 18)
  source 1..3395: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctatgcattacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggcggccgccctaggggatccatgcattacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 42 - DNA, 10 bp, synthetic construct
  source 1..10: mol_type = other DNA
gccaccatgg

SEQ ID NO: 43-44 - skipped (2 entries)

SEQ ID NO: 45 - protein, 735 aa, synthetic construct
  REGION 1..735: note = Synthetic: SL65
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 46 - DNA, 2208 bp, synthetic construct
  misc_feature 1..2208: note = Synthetic: SL65 coding sequence
  source 1..2208: mol_type = other DNA
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccccagtacggctacctaacactcaacaacggtagtcaggccgtgggacgctcctccttttactgcctggaatatttcccatcgcagatgctgagaacgggcaataactttgagttcagctacagcttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttaccttacccgtcccctgtaa

SEQ ID NO: 47-50 - skipped (4 entries)

SEQ ID NO: 51 - DNA, 3242 bp, synthetic construct
  misc_feature 1..3242: note = Synthetic: Construct 1B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), hGLA signal peptide (SEQ ID 7), mature peptide (SEQ ID 2), bGH polyA (SEQ ID NO 23), 3ITR (SEQ ID NO: 18)
  source 1..3242: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 52 - DNA, 3242 bp, synthetic construct
  misc_feature 1..3242: note = Synthetic: Construct 2B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), GLA coA signal peptide (SEQ ID 8), coA mature peptide (SEQ ID 3), bGH polyA (SEQ ID NO 23), 3ITR (SEQ ID NO: 18)
  source 1..3242: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctgagaaatcctgagctgcacctgggctgtgccctggctctgagatttctggctctggtgtcctgggacatccctggcgctagagccctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctatgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 53 - DNA, 3242 bp, synthetic construct
  misc_feature 1..3242: note = Synthetic: Construct 3B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), GLA coB signal peptide (SEQ ID 9), coB mature peptide (SEQ ID 4), bGH polyA (SEQ ID NO 23), 3ITR (SEQ ID NO: 18)
  source 1..3242: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctcagaaacccagaactccatctggggtgcgctctcgccctccgcttcctggcactcgtcagctgggatattcctggggctagagccctcgataatgggctcgctcggactccaaccatgggctggctccactgggaacgcttcatgtgtaatctggactgccaagaagaaccagatagctgtatcagcgaaaaactgtttatggaaatggctgagctcatggtcagcgagggctggaaggacgccggctacgagtatctgtgtatcgacgactgctggatggcccctcagcgggattcagagggcaggctccaggctgacccacagcggtttccccacggaattcggcagctggccaattacgtgcacagcaaaggcctgaagctggggatctacgctgatgtggggaacaagacctgcgctggatttccaggcagcttcggctactacgatatcgacgcacagaccttcgccgattggggcgtcgacctcctgaaatttgatggatgctattgcgatagcctcgaaaacctggccgacgggtacaaacacatgagcctcgctctgaaccggaccggccggagcatcgtgtacagctgcgaatggcctctgtacatgtggcccttccagaagccaaattacacagagattagacagtattgcaatcattggcggaacttcgccgacatcgatgacagctggaagagcatcaagagcatcctcgactggacctcattcaatcaggaacgcattgtggacgtggcaggaccaggcgggtggaatgatcccgacatgctggtgattggcaactttggactgagctggaatcagcaggtcacccagatggcactgtgggctatcatggccgcccccctgtttatgagcaacgacctcaggcacatctcaccacaagccaaggcactgctccaggataaggatgtcattgcaattaatcaggatccactgggcaaacaaggctatcagctgagacagggagataatttcgaagtgtgggagcggcctctgagcggactggcatgggccgtggctatgatcaacagacaggagatcggcggcccaagatcatacacaatcgccgtggcatcactgggcaagggagtcgcctgcaaccccgcatgctttatcactcagctgctgcctgtgaaaaggaagctgggcttctacgaatggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 54 - DNA, 3242 bp, synthetic construct
  misc_feature 1..3242: note = Synthetic: Construct 4B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), GLA coB-CpG signal peptide (SEQ ID 10), coB-CpG mature peptide (SEQ ID 5), bGH polyA (SEQ ID NO 23), 3ITR (SEQ ID NO: 18)
  source 1..3242: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgcagctcagaaacccagaactccatctggggtgtgccctggccctgaggttcctggcactggtgagctgggatattcctggggctagagccctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 55 - DNA, 3206 bp, synthetic construct
  misc_feature 1..3206: note = Synthetic: Construct 5B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 signal peptide (SEQ ID 11 and 12), mature peptide (SEQ ID 2), bGH polyA (SEQ ID NO 23), 3ITR (SEQ ID NO: 18)
  source 1..3206: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatgggctggagctgcatcatcctgttcctggtggccaccgccaccggcgtgcacagcctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 56 - DNA, 3206 bp, synthetic construct
  misc_feature 1..3206: note = Synthetic: Construct 6B - 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC signal peptide (SEQ ID 13), coA mature peptide (SEQ ID 3), bGH polyA (SEQ ID NO 23), 3ITR (SEQ ID NO: 18)
  source 1..3206: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtcgctaccgcaaccggagtgcacagcctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctatgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 57 - DNA, 3206 bp, synthetic construct
  misc_feature 1..3206: note = Synthetic: Construct 7B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC signal peptide (SEQ ID 13), coB mature peptide (SEQ ID 4), bGH polyA (SEQ ID NO 23), 3ITR (SEQ ID NO: 18)
  source 1..3206: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtcgctaccgcaaccggagtgcacagcctcgataatgggctcgctcggactccaaccatgggctggctccactgggaacgcttcatgtgtaatctggactgccaagaagaaccagatagctgtatcagcgaaaaactgtttatggaaatggctgagctcatggtcagcgagggctggaaggacgccggctacgagtatctgtgtatcgacgactgctggatggcccctcagcgggattcagagggcaggctccaggctgacccacagcggtttccccacggaattcggcagctggccaattacgtgcacagcaaaggcctgaagctggggatctacgctgatgtggggaacaagacctgcgctggatttccaggcagcttcggctactacgatatcgacgcacagaccttcgccgattggggcgtcgacctcctgaaatttgatggatgctattgcgatagcctcgaaaacctggccgacgggtacaaacacatgagcctcgctctgaaccggaccggccggagcatcgtgtacagctgcgaatggcctctgtacatgtggcccttccagaagccaaattacacagagattagacagtattgcaatcattggcggaacttcgccgacatcgatgacagctggaagagcatcaagagcatcctcgactggacctcattcaatcaggaacgcattgtggacgtggcaggaccaggcgggtggaatgatcccgacatgctggtgattggcaactttggactgagctggaatcagcaggtcacccagatggcactgtgggctatcatggccgcccccctgtttatgagcaacgacctcaggcacatctcaccacaagccaaggcactgctccaggataaggatgtcattgcaattaatcaggatccactgggcaaacaaggctatcagctgagacagggagataatttcgaagtgtgggagcggcctctgagcggactggcatgggccgtggctatgatcaacagacaggagatcggcggcccaagatcatacacaatcgccgtggcatcactgggcaagggagtcgcctgcaaccccgcatgctttatcactcagctgctgcctgtgaaaaggaagctgggcttctacgaatggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 58 - DNA, 3206 bp, synthetic construct
  misc_feature 1..3206: note = Synthetic: Construct 8B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), bGH polyA (SEQ ID NO 23), 3ITR (SEQ ID NO: 18)
  source 1..3206: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaatcattaactacagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 59 - DNA, 3823 bp, synthetic construct
  misc_feature 1..3823: note = Synthetic: Construct 9B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), WPRE (SEQ ID NO 25),bGH polyA (SEQ ID NO 23), 3ITR (SEQ ID NO: 18)
  source 1..3823: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctatttgtgaaatttgtgatgctattgctttatttgtaaccatctactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 60 - DNA, 3729 bp, synthetic construct
  misc_feature 1..3729: note = Synthetic: Construct 10B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), WPRE (SEQ ID NO 25),SV40 polyA (SEQ ID NO 24), 3ITR (SEQ ID NO: 18)
  source 1..3729: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 61 - DNA, 3391 bp, synthetic construct
  misc_feature 1..3391: note = Synthetic: Construct 11B- 5ITR (SEQ ID NO:17), Apo E/C-I (SEQ ID NO: 19), A1AT (SEQ ID NO: 20), Beta-globin intron (SEQ ID NO: 21) , KOZAK consensus leader sequence (SEQ ID NO: 22), IgG1 coC-CpG signal peptide (SEQ ID 14), coB-CpG mature peptide (SEQ ID 5), W3SL (SEQ ID NO 26),SV40 polyA (SEQ ID NO 24), 3ITR (SEQ ID NO: 18)
  source 1..3391: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaggacgtcccctgcaggcagtgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtcgccgccaccatggggtggtcatgtattatcctgtttctggtggccacagccacaggcgtgcacagcctggacaatgggctggccaggactccaaccatgggctggctccactgggagaggttcatgtgtaatctggactgccaagaagaaccagatagctgtatctctgagaaactgtttatggaaatggctgagctcatggtcagcgagggctggaaggatgccggctacgagtatctgtgtatcgatgactgctggatggcccctcagagggattcagagggcaggctccaggctgacccacagaggtttccccatggcatcaggcagctggccaattacgtgcacagcaaaggcctgaagctggggatctatgccgatgtggggaacaagacctgtgccggatttccaggcagctttggctactatgacatcgatgcccagacctttgccgattggggggtggatctcctgaaatttgatggatgctattgtgacagcctggagaacctggccgatggctacaaacacatgagcctggccctgaacaggaccggcaggagcatcgtgtacagctgtgagtggcctctgtacatgtggcccttccagaagccaaattacacagagattagacagtattgcaatcattggaggaactttgccgacattgacgacagctggaagagcatcaagagcatcctggactggacctcattcaatcaggagaggattgtggatgtggcaggaccagggggctggaatgatcctgacatgctggtgattggcaactttggactgagctggaatcagcaggtcacccagatggcactgtgggctatcatggccgcccccctgtttatgagcaatgacctcaggcacatctcaccacaagccaaggcactgctccaggataaggatgtcattgcaattaatcaggatccactgggcaaacaaggctatcagctgagacagggagataattttgaggtgtgggagaggcctctgtctggcctggcatgggccgtggctatgatcaacagacaggagatcggcggcccaagatcatacacaatcgccgtggcatcactgggcaagggagtggcctgcaaccctgcctgctttatcactcagctgctgcctgtgaaaaggaagctgggcttctatgagtggacaagcagactgaggagccacatcaaccccacaggaacagtcctgctccaactggaaaatactatgcaaatgtcactgaaagatctgctgtgagctagctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggcggccgccctaggggatccatgcattacgtgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2024/0424141 A1
(43) Pub. Date: Dec. 26, 2024

(54) AAV PARTICLES COMPRISING A LIVER-TROPIC CAPSID PROTEIN AND ALPHA-GALACTOSIDASE AND THEIR USE TO TREAT FABRY DISEASE

(71) Applicants: CANbridge Pharmaceuticals, Inc., Cambridge, MA (US); LogicBio Therapeutics, Inc., Lexington, MA (US)

(72) Inventors: Yunxiang Zhu, Wayland, MA (US); Peter Pechan, Newton, MA (US); Anannya Banga, Lexington, MA (US); Susana Gordo Villoslada, Lexington, MA (US)

(21) Appl. No.: 18/686,355

(22) PCT Filed: Aug. 25, 2022
(86) PCT No.: PCT/US2022/075474

§ 371 Date: Feb. 23, 2024

Related U.S. Application Data
(60) Provisional application No. 63/237,122, filed on Aug. 25, 2021.

(51) Int. Cl. A61K 48/00; A61K 38/45; A61P 3/00; C12N 15/86

(52) U.S. Cl. CPC A61K 48/0058; A61K 38/45; A61P 3/00; C12N 15/86; C12Y 204/01082; C12N 2750/14122; C12N 2750/14143; C12N 2750/14151; C12N 2800/22; C12N 2830/008; C12N 2830/48; C12N 2830/50

(57) ABSTRACT

The present disclosure provides compositions comprising isolated, e.g., recombinant, adeno-associated viruses (AAV) particles, comprising a liver tropic capsid protein, e.g., an sL65 capsid protein, for delivery of a GALprotein. The present disclosure also provides compositions comprising a first nucleic acid encoding a liver tropic capsid protein, e.g., an sL65 capsid protein, and a second nucleic acid comprising a transgene encoding a GAL protein. The present disclosure also provides methods for making an isolated, e.g., recombinant, AAV particles, and methods for delivering an exogenous GALprotein into a subject and/or methods for treating a subject having a GAL-associated disease or disorder, e.g., a lysosomal storage disorder, e.g., Fabry disease.

### RELATED APPLICATION

The present application claims priority to U.S. Provisional Application No. 63/237,122, filed on Aug. 25, 2021, the entire contents of which are incorporated herein by reference.

### BACKGROUND

Lysosomal storage disorders are a group of autosomal recessive diseases caused by the accumulation of cellular glycosphingolipids, glycogen, or mucopolysaccharides, due to defective hydrolytic enzymes. Fabry disease is one of several lysosomal storage disorders caused by a deficiency in the enzyme alpha-galactosidase (α-GAL). This enzyme is necessary for the daily metabolism of glycosphingolipids in the body such as globotriaosylceramide (also known as GL-3 or GB-3) and related glycosphingolipids. When proper metabolism of this lipid and other similar lipids does not occur, GL-3 accumulates in the majority of cells throughout the body. The resulting progressive lipid accumulation leads to cell damage. The cell damage causes a wide range of mild to severe symptoms including potentially life-threatening consequences such as kidney failure, heart failure and strokes often at a relatively early age.

There are generally two types of Fabry disease which reflect a person's age when symptoms first appear. For the classic type, symptoms appear during childhood or the teenage years. One hallmark disease symptom, a painful burning sensation in the hands and feet, may be noticeable as early as age two. Symptoms get progressively worse over time. For late-onset/atypical type, people don't have symptoms until they're in their 30s or older. The first indication of a problem may be kidney failure or heart disease.

Treatment of Fabry disease is predominantly by enzyme replacement therapy (ERT) with recombinant GAL, e.g., with the product marketed as Fabrazyme® (Genzyme, Inc.) and Replagal® (TKT, Inc.). ERT typically involves intravenous, subcutaneous or intramuscular infusion of a purified form of the corresponding wild-type protein, or implantation of the protein in a bio-erodable solid form for extended-release. One of the main complications with ERT is attainment and maintenance of therapeutically effective amounts of protein due to rapid degradation of the infused protein. As a result, ERT requires numerous, high-dose infusions and as a result, is costly and time consuming. In addition, ERT therapy has several other caveats, such as difficulties with large-scale generation, purification and storage of properly folded protein, obtaining properly glycosylated native protein, generation of an anti-protein immune response in some patients, and failure of protein to cross the blood-brain barrier in sufficient quantities to affect diseases having significant central nervous system involvement.

Accordingly, there remains a long felt-need to develop new therapies to treat Fabry disease and to ameliorate GALdeficiencies in patients afflicted with GAL-associated disorders.

### SUMMARY OF THE DISCLOSURE

The present disclosure provides AAV-based compositions and methods for treating a GAL-associated disease in patients. Specifically, by utilizing a recombinant adeno-associated virus (rAAV) particle comprising a liver-tropic capsid protein, e.g., an sL65 capsid protein or an LK03 capsid protein, a superior and highly specific liver transduction and expression of a target protein, e.g., GAL, is achieved, thus making the rAAV particles of the present disclosure a promising gene therapy candidate for treating a GAL-associated disease, such as Fabry disease.

Accordingly, in one aspect, the present disclosure provides an isolated recombinant adeno-associated virus (rAAV) particle comprising an AAV capsid protein, wherein the capsid protein comprises an amino acid sequence of SEQ ID NO: 45, or an amino acid sequence at least 85% identical thereto, and a nucleic acid comprising a transgene encoding an alpha-galactosidase (GAL) protein.

In some embodiments, the nucleic acid encoding the capsid protein comprises a nucleotide sequence of SEQ ID NO: 46, or a nucleotide sequence at least 85% identical thereto.

In some embodiments, the encoded GAL protein comprises the amino acid sequence of SEQ ID NO: 1, or an amino acid sequence at least 85% identical thereto.

In some embodiments, the transgene encoding the GAL protein comprises the nucleotide sequence of SEQ ID NO: 2, or a nucleotide sequence at least 85% identical thereto.

In some embodiments, the transgene encoding the GAL protein is codon optimized.

In some embodiments, the transgene encoding the GAL protein comprises the nucleotide sequence of any one of SEQ ID NOs: 3-5, or a nucleotide sequence at least 85% identical thereto.

In some embodiments, the transgene encoding the GAL protein further encodes a signal sequence.

In some embodiments, the encoded signal sequence comprises the amino acid sequence of SEQ ID NO: 6, or an amino acid sequence at least 70% identical thereto.

In some embodiments, the encoded signal sequence is encoded by a nucleic acid comprising the nucleotide sequence of SEQ ID NO: 7, or a nucleotide sequence at least 70% identical thereto.

In some embodiments, the encoded signal sequence is encoded by a codon optimized nucleic acid.

In some embodiments, the codon optimized nucleic acid comprises the nucleotide sequence of any one of SEQ ID NOs: 8-10, or a nucleotide sequence at least 70% identical thereto.

In some embodiments, the encoded signal sequence comprises the amino acid sequence of SEQ ID NO: 11, or an amino acid sequence at least 70% identical thereto.

In some embodiments, the encoded signal sequence is encoded by a nucleic acid comprising the nucleotide sequence of SEQ ID NO: 12, or a nucleotide sequence at least 70% identical thereto.

In some embodiments, the encoded signal sequence is encoded by a codon optimized nucleic acid.

In some embodiments, the codon optimized nucleic acid comprises the nucleotide sequence of any one of SEQ ID NOs: 13-14, or a nucleotide sequence at least 70% identical thereto.

In some embodiments, the transgene encoding the GAL protein comprises in 5′ to 3′ order:
(i) a nucleotide sequence encoding a signal sequence comprising the nucleotide sequence of any one of SEQ ID NOs: 7-10, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto; or
(ii) a nucleotide sequence encoding a signal sequence comprising the nucleotide sequence of any one of SEQ ID NOs: 12-14, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto.

In some embodiments, the transgene encoding the GAL protein comprises in 5′ to 3′ order:
(i) a nucleotide sequence encoding a signal sequence comprising the nucleotide sequence of SEQ ID NO: 7, or a nucleotide sequence at least 70% identical thereto; and a nucleotide sequence encoding a GAL protein comprising the nucleotide sequence of SEQ ID NO: 2, or a nucleotide sequence at least 85% identical thereto;
(ii) a nucleotide sequence encoding a signal sequence comprising the nucleotide sequence of SEQ ID NO: 8, or a nucleotide sequence at least 70% identical thereto; and a nucleotide sequence encoding a GAL protein comprising the nucleotide sequence of SEQ ID NO: 3, or a nucleotide sequence at least 85% identical thereto;
(iii) a nucleotide sequence encoding a signal sequence comprising the nucleotide sequence of SEQ ID NO: 9, or a nucleotide sequence at least 70% identical thereto; and a nucleotide sequence encoding a GAL protein comprising the nucleotide sequence of SEQ ID NO: 4, or a nucleotide sequence at least 85% identical thereto;
(iv) a nucleotide sequence encoding a signal sequence comprising the nucleotide sequence of SEQ ID NO: 10, or a nucleotide sequence at least 70% identical thereto; and a nucleotide sequence encoding a GAL protein comprising the nucleotide sequence of SEQ ID NO: 5, or a nucleotide sequence at least 85% identical thereto;
(v) a nucleotide sequence encoding a signal sequence comprising the nucleotide sequence of SEQ ID NO: 12, or a nucleotide sequence at least 70% identical thereto; and a nucleotide sequence encoding a GAL protein comprising the nucleotide sequence of SEQ ID NO: 2;
(vi) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 3, or a nucleotide sequence at least 85% identical thereto;
(vii) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 4, or a nucleotide sequence at least 85% identical thereto; or
(viii) a nucleotide sequence encoding a signal sequence comprising the nucleotide sequence of SEQ ID NO: 14, or a nucleotide sequence at least 70% identical thereto; and a nucleotide sequence encoding a GAL protein comprising the nucleotide sequence of SEQ ID NO: 5, or a nucleotide sequence at least 85% identical thereto; In some embodiments, the transgene encoding the GAL protein encodes in 5′ to 3′ order:
(i) a signal sequence comprising the amino acid sequence of SEQ ID NO: 6, or an amino acid sequence at least 70% identical thereto; and a GAL protein comprising the amino acid sequence of SEQ ID NO: 1, or an amino acid sequence at least 85% identical thereto; or
(ii) a signal sequence comprising the amino acid sequence of SEQ ID NO: 11, or an amino acid sequence at least 70% identical thereto; and a GAL protein comprising the amino acid sequence of SEQ ID NO: 1, or an amino acid sequence at least 85% identical thereto.

In some embodiments, the isolated rAAV particle further comprises a promoter operably linked to the nucleic acid comprising the transgene encoding the GAL protein.

In some embodiments, the promoter comprises a tissue specific promoter or a ubiquitous promoter.

In some embodiments, the promoter comprises:
(i) an A1AT promoter, an EF-1a promoter, a chicken β-actin (CBA) promoter and/or its derivative CAG, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a 0-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter), a liver promoter, a skeletal muscle promoter or a fragment, e.g., a truncation, or a functional variant thereof; and/or
(ii) the nucleotide sequence of SEQ ID NOs: 20, or a nucleotide sequence at least 95% identical thereto.

In some embodiments, the isolated rAAV particle further comprises an inverted terminal repeat (ITR) sequence.

In some embodiments, the ITR sequence is positioned 5′ relative to the nucleic acid comprising the transgene encoding the GAL protein.

In some embodiments, the ITR sequence is positioned 3′ relative to the nucleic acid comprising the transgene encoding the GAL protein.

In some embodiments, the isolated rAAV particle comprises an ITR sequence positioned 5′ relative to the nucleic acid comprising the transgene encoding the GAL protein and an ITR sequence positioned 3′ relative to the nucleic acid comprising the transgene encoding the GAL protein.

In some embodiments, the ITR sequence comprises a nucleotide sequence of SEQ ID NO: 17 and/or 18, or a nucleotide sequence at least 95% identical thereto.

In some embodiments, the isolated rAAV particle further comprises an enhancer.

In some embodiments, the enhancer comprises the nucleotide sequence of SEQ ID NO: 19, or a nucleotide sequence at least 95% identical thereto.

In some embodiments, the isolated rAAV particle further comprises an intron region.

In some embodiments, the intron region comprises the nucleotide sequence of SEQ ID NO:21, or a nucleotide sequence at least 95% identical thereto.

In some embodiments, the isolated rAAV particle further comprises a Kozak sequence.

In some embodiments, the Kozak sequence comprises the nucleotide sequence of SEQ ID NO: 22, or a nucleotide sequence at least 95% identical thereto.

In some embodiments, the isolated rAAV particle further comprises a polyadenylation (polyA) signal region.

In some embodiments, the polyA signal region comprises the nucleotide sequence of SEQ ID NO:23, or a nucleotide sequence at least 95% identical thereto.

In some embodiments, the polyA signal region comprises the nucleotide sequence of SEQ ID NO:24, or a nucleotide sequence at least 95% identical thereto.

In some embodiments, the isolated rAAV particle further comprises a Woodchuck Hepatitis Virus Posttranscriptional Regulatory Element (WPRE) sequence.

In some embodiments, the WPRE sequence comprises the nucleotide sequence of SEQ ID NO: 25, or a nucleotide sequence at least 95% identical thereto. In some embodiments, the WPRE sequence comprises the nucleotide sequence of SEQ ID NO: 26, or a nucleotide sequence at least 95% identical thereto.

In some embodiments, the isolated rAAV particle comprises in 5′ to 3′ order, a 5′ ITR sequence, an enhancer, a promoter sequence, a Kozak sequence, a nucleotide sequence encoding a signal sequence, a nucleotide sequence encoding a GAL protein, a polyA signal region, and a 3′ ITR sequence.

In some embodiments, the isolated rAAV particle comprises in 5′ to 3′ order, a 5′ ITR sequence, an enhancer, a promoter sequence, a Kozak sequence, a nucleotide sequence encoding a signal sequence, a nucleotide sequence encoding a GAL protein, a WPRE sequence, a polyA signal region, and a 3′ ITR sequence.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 7, or a nucleotide sequence at least 85% identical thereto;
(vii) a nucleotide sequence encoding a GAL protein comprising the nucleotide sequence of SEQ ID NO: 2 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto;

In some embodiments, the isolated rAAV particle comprises in 5′ to 3′ order
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 8, or a nucleotide sequence at least 85% identical thereto;
(vii) a nucleotide sequence encoding a GAL protein comprising the nucleotide sequence of SEQ ID NO: 3 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 9, or a nucleotide sequence at least 85% identical thereto;
(vii) a nucleotide sequence encoding a GAL protein comprising the nucleotide sequence of SEQ ID NO: 4 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 10, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 12, or a nucleotide sequence at least 85% identical thereto;
(vii) a nucleotide sequence encoding a GAL protein comprising the nucleotide sequence of SEQ ID NO: 2 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 13, or a nucleotide sequence at least 85% identical thereto;
(vii) a nucleotide sequence encoding a GAL protein comprising the nucleotide sequence of SEQ ID NO: 3 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 13, or a nucleotide sequence at least 85% identical thereto;
(vii) a nucleotide sequence encoding a GAL protein comprising the nucleotide sequence of SEQ ID NO: 4 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order,
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a WPRE sequence comprising the nucleotide sequence of SEQ ID NO: 25, or a nucleotide sequence at least 95% identical thereto;
(ix) a polyadenylation sequence comprising the nucleotide sequence of SEQ ID NO: 23, or a nucleotide sequence at least 95% identical thereto; and
(x) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a WPRE sequence comprising the nucleotide sequence of SEQ ID NO: 25, or a nucleotide sequence at least 95% identical thereto;
(ix) a polyadenylation sequence comprising the nucleotide sequence of SEQ ID NO: 24, or a nucleotide sequence at least 95% identical thereto; and
(x) a 3′ ITR sequence region comprising the nucleotide sequence of SEQ ID NO: 18, or a nucleotide sequence at least 95% identical thereto.

In some embodiments, the isolated rAAV particle comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a WPRE sequence comprising the nucleotide sequence of SEQ ID NO: 26, or a nucleotide sequence at least 95% identical thereto;
(ix) a polyadenylation sequence comprising the nucleotide sequence of SEQ ID NO: 24, or a nucleotide sequence at least 95% identical thereto; and
(x) a 3′ ITR sequence region comprising the nucleotide sequence of SEQ ID NO: 18, or a nucleotide sequence at least 95% identical thereto.

In one aspect, the present invention provides a composition comprising a first nucleic acid encoding an AAV capsid protein, wherein the capsid protein comprises an amino acid sequence of SEQ ID NO: 45, or an amino acid sequence at least 85% identical thereto, and a second nucleic acid comprising a transgene encoding an alpha-glucosidase (GAL) protein.

In some embodiments, the first nucleic acid encoding the capsid protein comprises a nucleotide sequence of SEQ ID NO: 46, or a nucleotide sequence at least 85% identical thereto.

In some embodiments, the encoded GAL protein comprises the amino acid sequence of SEQ ID NO: 1, or an amino acid sequence at least 85% identical thereto.

In some embodiments, the transgene encoding the GAL protein comprises the nucleotide sequence of SEQ ID NO: 2, or a nucleotide sequence at least 85% identical thereto.

In some embodiments, the transgene encoding the GAL protein is codon optimized.

In some embodiments, the transgene encoding the GAL protein comprises the nucleotide sequence of any one of SEQ ID NOs: 3-5, or a nucleotide sequence at least 85% identical thereto.

In some embodiments, the transgene encoding the GAL protein further encodes a signal sequence.

In some embodiments, the encoded signal sequence comprises the amino acid sequence of SEQ ID NO: 6, or an amino acid sequence at least 70% identical thereto.

In some embodiments, the encoded signal sequence is encoded by a nucleic acid comprising the nucleotide sequence of SEQ ID NO: 7, or a nucleotide sequence at least 70% identical thereto.

In some embodiments, the encoded signal sequence is encoded by a codon optimized nucleic acid.

In some embodiments, the codon optimized nucleic acid comprises the nucleotide sequence of any one of SEQ ID NOs: 8-10, or a nucleotide sequence at least 70% identical thereto.

In some embodiments, the encoded signal sequence comprises the amino acid sequence of SEQ ID NO: 11, or an amino acid sequence at least 70% identical thereto.

In some embodiments, the encoded signal sequence is encoded by a nucleic acid comprising the nucleotide sequence of SEQ ID NO: 12, or a nucleotide sequence at least 70% identical thereto.

In some embodiments, the encoded signal sequence is encoded by a codon optimized nucleic acid.

In some embodiments, the codon optimized nucleic acid comprises the nucleotide sequence of any one of SEQ ID NOs: 13-14, or a nucleotide sequence at least 70% identical thereto.

In some embodiments, the transgene encoding the GAL protein comprises in 5′ to 3′ order:
(i) a nucleotide sequence encoding a signal sequence comprising the nucleotide sequence of any one of SEQ ID NOs: 7-10, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto; or
(ii) a nucleotide sequence encoding a signal sequence comprising the nucleotide sequence of any one of SEQ ID NOs: 12-14, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto.

In some embodiments, the transgene encoding the GAL protein comprises in 5′ to 3′ order:
(i) a nucleotide sequence encoding a signal sequence comprising the nucleotide sequence of SEQ ID NO: 7, or a nucleotide sequence at least 70% identical thereto; and a nucleotide sequence encoding a GAL protein comprising the nucleotide sequence of SEQ ID NO: 2, or a nucleotide sequence at least 85% identical thereto;
(ii) a nucleotide sequence encoding a signal sequence comprising the nucleotide sequence of SEQ ID NO: 8, or a nucleotide sequence at least 70% identical thereto; and a nucleotide sequence encoding a GAL protein comprising the nucleotide sequence of SEQ ID NO: 3, or a nucleotide sequence at least 85% identical thereto;
(iii) a nucleotide sequence encoding a signal sequence comprising the nucleotide sequence of SEQ ID NO: 9, or a nucleotide sequence at least 70% identical thereto; and a nucleotide sequence encoding a GAL protein comprising the nucleotide sequence of SEQ ID NO: 4, or a nucleotide sequence at least 85% identical thereto;
(iv) a nucleotide sequence encoding a signal sequence comprising the nucleotide sequence of SEQ ID NO: 10, or a nucleotide sequence at least 70% identical thereto; and a nucleotide sequence encoding a GAL protein comprising the nucleotide sequence of SEQ ID NO: 5, or a nucleotide sequence at least 85% identical thereto;
(v) a nucleotide sequence encoding a signal sequence comprising the nucleotide sequence of SEQ ID NO: 12, or a nucleotide sequence at least 70% identical thereto; and a nucleotide sequence encoding a GAL protein comprising the nucleotide sequence of SEQ ID NO: 2;
(vi) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 3, or a nucleotide sequence at least 85% identical thereto;
(vii) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 4, or a nucleotide sequence at least 85% identical thereto; or
(vi) a nucleotide sequence encoding a signal sequence comprising the nucleotide sequence of SEQ ID NO: 14, or a nucleotide sequence at least 70% identical thereto; and a nucleotide sequence encoding a GAL protein comprising the nucleotide sequence of SEQ ID NO: 5, or a nucleotide sequence at least 85% identical thereto;

In some embodiments, the transgene encoding the GAL protein encodes in 5′ to 3′ order:
(i) a signal sequence comprising the amino acid sequence of SEQ ID NO: 6, an amino acid sequence at least 70% identical thereto; and a GAL protein comprising the amino acid sequence of SEQ ID NO: 1, or an amino acid sequence at least 85% identical thereto; or
(ii) a signal sequence comprising the amino acid sequence of SEQ ID NO: 11, an amino acid sequence at least 70% identical thereto; and a GAL protein comprising the amino acid sequence of SEQ ID NO: 1, or an amino acid sequence at least 85% identical thereto.

In one aspect, the present invention provides an isolated nucleic acid comprising a transgene encoding an alpha-glucosidase (GAL) protein, wherein the transgene encoding the GAL protein comprises the nucleotide sequence of any one of SEQ ID NOs: 3-5, or a nucleotide sequence at least 85% identical thereto.

In some embodiments, the transgene further encodes a signal sequence.

In some embodiments, the encoded signal sequence is encoded by a nucleic acid comprising a nucleotide sequence of any one of SEQ ID NOs: 8-10, or a nucleotide sequence at least 70% identical thereto.

In some embodiments, the encoded signal sequence is encoded by a nucleic acid comprising a nucleotide sequence of any one of SEQ ID NOs: 12-14, or a nucleotide sequence at least 70% identical thereto.

In another aspect, the present invention provides a composition comprising the nucleic acid of the invention.

In one aspect, the present invention provides a cell comprising the isolated rAAV particle of the invention, or the composition of the invention, or the nucleic acid of the invention.

In some embodiments, the cell is a mammalian cell, an insect cell, or a bacterial cell.

In one aspect, the present invention provides a method of making an isolated recombinant adeno-associated virus (rAAV) particle, the method comprising (i) providing a host cell comprising a nucleic acid comprising a transgene encoding an alpha-glucosidase (GAL) protein; and (ii) incubating the host cell under conditions suitable to enclose the transgene in an AAV capsid protein, wherein the capsid protein comprises an amino acid sequence of SEQ ID NO: 45, or an amino acid sequence at least 85% identical thereto; thereby making the isolated rAAV particle.

In another aspect, the present invention provides a method of making an isolated recombinant adeno-associated virus (rAAV) particle, the method comprising (i) providing a host cell comprising a first nucleic acid comprising a transgene encoding an alpha-glucosidase (GAL) protein; (ii) introducing into the host cell a second nucleic acid encoding an AAV capsid protein, wherein the capsid protein comprises an amino acid sequence of SEQ ID NO: 45, or an amino acid sequence at least 85% identical thereto; and (iii) incubating the host cell under conditions suitable to enclose the transgene in the AAV capsid protein; thereby making the isolated rAAV particle.

In some embodiments, the host cell comprises a mammalian cell, an insect cell or a bacterial cell.

In one aspect, the present invention provides a pharmaceutical composition comprising an rAAV particle of the invention, and a pharmaceutically acceptable excipient.

In one aspect, the present invention provides a method of delivering an exogenous GAL protein to a subject, comprising administering an effective amount of the pharmaceutical composition of the invention, or the isolated rAAV particle of the invention, thereby delivering the exogenous GAL to the subject.

In some embodiments, the subject has, has been diagnosed with having, or is at risk of having a GAL-associated disease.

In some embodiments, the GAL-associated disease is a lysosomal storage disease.

In another aspect, the present invention provides a method of treating a subject having or diagnosed with having a GAL-associated disease comprising administering an effective amount of the pharmaceutical composition of the invention, or the isolated rAAV particle of the invention, thereby treating the GAL-associated disease in the subject.

In one aspect, the present invention provides a method of treating a subject having or diagnosed with having a lysosomal storage disease, comprising administering an effective amount of the pharmaceutical composition of the invention, or the isolated rAAV particle of the invention, thereby treating the lysosomal storage disease in the subject.

In some embodiments, the GAL-associated disease or the lysosomal storage disease is Fabry disease.

In one aspect, the present invention provides an isolated recombinant adeno-associated virus (rAAV) particle comprising an AAV viral genome comprising or consisting of the nucleic acid sequence of any one of SEQ ID NO: 31-41 and 51-61, and a capsid protein comprising the amino acid sequence of SEQ ID NO: 45.

In another aspect, the present invention provides an isolated recombinant viral genome comprising or consisting of the nucleic acid sequence of any one of SEQ ID NO: 31-41, and 51-61.

The details of various aspects or embodiments of the present disclosure are set forth below. Other features, objects, and advantages of the disclosure will be apparent from the description and the claims. In the description, the singular forms also include the plural unless the context clearly dictates otherwise. Unless defined otherwise, all technical and scientific terms used herein have the same meaning as commonly understood by one of ordinary skill in the art in the field of this disclosure. In the case of conflict, the present description will control.

### BRIEF DESCRIPTION OF THE DRAWINGS

FIG. 1 provides the schematic of exemplary constructs encoding a human alpha galactosidase (GAL) protein and a signal peptide.

FIG. 2 depicts the schematic of AAV-GLA construct combinations. Shown are the AAV genomes with various combinations of native and codon-optimized signal peptides and mature peptides of human alpha galactosidase (GAL) under control of ApoE-A1AT liver-specific promoter and bGH poly-adenylation sequence. 1-8=Constructs #1-8; ITR=AAV2 inverted terminal repeats; ApoE=Apo E/C-I HCR-1 enhancer; A1AT=A1AT promoter; bgi=beta-globin intron; bGH A+=bovine growth hormone poly-adenylation sequence; sp=signal peptide; hGLA=native human GAL; coA-GLA=codon optimized version A of sp and GAL; coB-GLA=codon optimized version B of sp and GAL; coB-CpG-GLA=CpG reduced codon optimized version B of sp and GAL; sp-IgG1=reverse translated sequence of human IgG1 signal peptide; coC-IgG1=codon optimized version C of IgG1 signal peptide; sp-coC-CpG-IgG1=CpG reduced codon optimized version C of IgG1 signal peptide.

FIG. 3 depicts the Western blot visualization of GAL mature peptide in supernatants and precursor and mature peptide in lysates harvested from transfected HepG2 cells with plasmids 72 hrs post-transfection. Constructs #1-8 (specifically, constructs 1A-8A), expressing native GLA and codon-optimized GLA.

FIG. 4 depicts the quantification of GAL mature peptide by ELISA in supernatants and lysates harvested from transfected HepG2 cells with plasmids 72 hrs post-transfection. Constructs #1-8 (specifically, constructs 1A-8A), expressing native GLA and codon-optimized GLA.

FIG. 5 depicts the assessment of GAL protein activity in supernatants harvested from transfected HepG2 cells with plasmids 72 hrs post-transfection. Constructs #1-8 (specifically, constructs 1A-8A), expressing native GLA and codon-optimized GLA.

FIG. 6 depicts the assessment of GAL protein activity in plasma from Fabry mice Activity was assessed in samples harvested at the basal time point (day 0) and post-injection (day 7).

FIG. 7 depicts exemplary AAV-GLA construct combinations. Shown are the AAV genomes with various combinations of native and codon-optimized signal peptides and mature peptides of human alpha galactosidase (GAL). 1-11=Constructs #1-11; ITR=AAV2 inverted terminal repeats; bGH polyA=bovine growth hormone poly-adenylation sequence; sp=signal peptide; hGLA=native human GAL; coA-GLA=codon optimized version A of sp and GAL; coB-GLA=codon optimized version B of sp and GAL; coB-CpG-GLA=CpG reduced codon optimized version B of sp and GAL; sp-IgG1=reverse translated sequence of human IgG1 signal peptide; sp-IgG1co=codon optimized version C of IgG1 signal peptide; sp-IgG1coCpG=CpG reduced codon optimized version C of IgG1 signal peptide; WPRE=Woodchuck Hepatitis Virus Posttranscriptional Regulatory Element; W3SL=internally truncated WPRE sequence; SV40=SV40 polyA sequence.

FIG. 8 depicts the Western blot visualization of GAL mature peptide in supernatants and precursor and mature peptide in lysates harvested from transfected HepG2 cells with plasmids 72 hrs post-transfection. Constructs #1-11 (specifically, constructs 1B-11B), expressing native GLA and codon-optimized GLA.

FIG. 9 depicts the assessment of GAL protein activity in supernatants harvested from transfected HepG2 cells with plasmids 72 hrs post-transfection. Constructs #1-11 (specifically, constructs 1B-11B), expressing native GLA and codon-optimized GLA.

FIG. 10 depicts the Western blot visualization of GAL mature peptide in supernatants harvested from HepG2 cells transduced with selected constructs packaged into AAV/DJ at three different MOIs, i.e., 5E4, 1D5 and 2E5.

FIG. 11 depicts the assessment of GAL protein activity in supernatants harvested from HepG2 cells transduced with selected constructs packaged into AAV/DJ at three different MOIs, i.e., 5E4, 1D5 and 2E5.

FIG. 12 depicts the assessment of GAL protein activity (left) and lyso-GB3 clearance (right) in plasma samples from Fabry mice. Fabry mice (n=5 per group) were administered with codon optimized constructs (constructs 2, 4, 6, 7, 8 and 9) in AAV-DJ at a dose of 5E11 vg/kg. GAL activity and lyso-GB3 levels were assessed in samples harvested at week 6.

FIG. 13 depicts the correlation between the plasma GAL activity and plasma lyso-GB3 levels.

FIG. 14 depicts the assessment of GAL protein activity in plasma samples from Fabry mice. Fabry mice (n=4 per group) were administered with codon optimized constructs (constructs 6 and 9) in AAV-DJ at a dose of 1E13 vg/kg. GAL activity levels were assessed in samples harvested pre-dose, and at week 1, week 4 and week 6.

FIG. 15 depicts the assessment of GAL protein activity (left) and albumin (right) levels in plasma samples from PXB mice. PXB mice (n=4 per group) were administered with codon optimized constructs (constructs 6 and 9) in AAV-SL65 at a dose of 3E13 vg/kg. GAL activity and albumin levels were assessed in samples harvested pre-dose, and at week 2, and week 4.

FIG. 16 depicts the plasma GAL protein activity normalized by the albumin control.

### DETAILED DESCRIPTION OF THE DISCLOSURE

The present disclosure provides compositions comprising isolated, e.g., recombinant, viral particles, e.g., adeno-associated virus (AAV) particles, comprising a liver tropic capsid protein, e.g., an sL65 capsid protein or an LK03 capsid protein, for delivery of a target protein, e.g., a GAL protein, and methods for delivering an exogenous GAL protein in a subject, and/or methods for treating a subject having a GAL-associated disease or disorder, e.g., a lysosomal storage disorder, e.g., Fabry disease, using the AAV particles of the disclosure.

The present disclosure also provides compositions comprising a first nucleic acid encoding an AAV capsid protein, e.g., an sL65 capsid protein, wherein the capsid protein comprises an amino acid sequence of SEQ ID NO: 45, or an amino acid sequence at least 85% identical thereto, and a second nucleic acid comprising a transgene encoding a GAL protein.

Adeno-associated viruses are small non-enveloped icosahedral capsid viruses of the Parvoviridae family characterized by a single stranded DNA viral genome. Parvoviridae family viruses consist of two subfamilies: Parvoviridae, which infect vertebrates, and Denoviridae, which infect invertebrates. AAV are capable of replication in vertebrate hosts including, but not limited to, human, primate, bovine, canine, equine, and ovine species. The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be modified to contain a minimum of components for the assembly of a functional recombinant virus, or viral particle, which is loaded with or engineered to express or deliver a desired nucleic acid construct or payload, e.g., a transgene, polypeptide-encoding polynucleotide, e.g., a GAL protein, which may be delivered to a target cell, tissue, or organism. In some embodiments, the target cell is a hepatic cell. In some embodiments, the target tissue is a hepatic tissue.

Gene therapy presents an alternative approach for Fabry disease. AAVs are commonly used in gene therapy approaches as a result of a number of advantageous features. Without wishing to be bound by theory, it is believed that in some embodiments, the AAV particles described herein can be used to administer and/or deliver a GAL protein (e.g., GAL and related proteins), in order to achieve sustained and high concentrations, allowing for longer lasting efficacy, fewer dose treatments, broad biodistribution, and/or more consistent levels of the GAL protein, relative to a non-AAV therapy.

The compositions and methods described herein provide improved features compared to prior enzyme replacement approaches, including (i) increased GAL activity in a cell, tissue, (e.g., a liver cell or tissue); (ii) increased and uniform biodistribution throughout the liver, and/or (iii) elevated payload expression, e.g., GLA mRNA expression, in liver. The compositions and methods described herein can be used in the treatment of disorders associated with a lack of a GAL protein and/or GAL activity, such as lysosomal storage diseases, e.g., Fabry disease.

### I. Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

Alpha-galactosidase (GAL): As used herein, the term “alpha-galactosidase (GAL)”, also known as α-GAL, GALA, galactosylgalactosylglucosylceramidase, gelibiase, alpha-D-galactosidase A, alpha-D-galactoside galactohydrolase 1, agalsidase alfa, alpha-gal A, and agalsidase, refers to a lysosomal enzyme which hydrolyses the terminal alpha-galactosyl moieties from glycolipids and glycoproteins. As used herein, the terms “GAL”, “GAL protein,” “GAL enzyme,” “α-GAL”, “alpha-GAL”, and the like refer to protein products or portions of protein products including peptides of the GLA gene (Ensemble gene ID: ENSG00000102393), homologs or variants thereof, and orthologs thereof, including non-human proteins and homologs thereof. GAL proteins include fragments, derivatives, and modifications of GLA gene products. Exemplary amino acid and nucleotide sequences of human GLA are shown in Table 1.

Adeno-associated virus (AAV): As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to a particle or a virion comprising an AAV capsid, e.g., an AAV capsid variant, and a polynucleotide, e.g., a viral genome. In some embodiments, the viral genome of the AAV particle comprises at least one payload region and at least one ITR. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. In some embodiments, the AAV particle may comprises a peptide, e.g., targeting peptide, present, e.g., inserted into, the capsid to enhance tropism for a desired target tissue. It is to be understood that reference to the AAV particle of the disclosure also includes pharmaceutical compositions thereof, even if not explicitly recited.

AAV vector: As used herein, the term “AAV vector” or “AAV construct” refers to a vector derived from an adeno-associated virus serotype. “AAV vector” refers to a vector that includes AAV nucleotide sequences as well as heterologous nucleotide sequences. AAV vectors require only the 145 base terminal repeats in cis to generate virus. All other viral sequences are dispensable and may be supplied in trans (Muzyczka (1992) Curr. Topics Microbiol. Immunol. 158:97-129). Typically, the rAAV vector genome will only retain the inverted terminal repeat (ITR) sequences so as to maximize the size of the transgene that can be efficiently packaged by the vector. The ITRs need not be the wild-type nucleotide sequences, and may be altered, e.g., by the insertion, deletion or substitution of nucleotides, as long as the sequences provide for functional rescue, replication and packaging.

Administering: As used herein, the term “administering” to a subject includes dispensing, delivering or applying a composition of the disclosure to a subject by any suitable route for delivery of the composition to the desired location in the subject. Alternatively or in combination, delivery is by the topical, parenteral or oral route, intracerebral injection, intramuscular injection, subcutaneous/intradermal injection, intravenous injection, buccal administration, transdermal delivery and administration by the rectal, colonic, vaginal, intranasal or respiratory tract route.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the capsid protein is an sL65 capsid protein, as described herein.

Codon optimization: As used herein, the term “codon optimization” refers to a process of changing codons of a given gene in such a manner that the polypeptide sequence encoded by the gene remains the same while the changed codons improve the process of expression of the polypeptide sequence. For example, if the polypeptide is of a human protein sequence and expressed in E. coli, expression will often be improved if codon optimization is performed on the DNA sequence to change the human codons to codons that are more effective for expression in E. coli.

Contacting: As used herein, the term “contacting” (i.e., contacting a cell with an agent) is intended to include incubating the agent and the cell together in vitro (e.g., adding the agent to cells in culture) or administering the agent to a subject such that the agent and cells of the subject are contacted in vivo. The term “contacting” is not intended to include exposure of cells to an agent that may occur naturally in a subject (i.e., exposure that may occur as a result of a natural physiological process).

GAL-associated disorder: The terms “GAL-associated disorder,” “GAL-associated disease,” and the like refer to diseases or disorders having a deficiency in the GLA gene, such as a heritable, e.g., X-linked, mutation in GLA resulting in deficient or defective GAL protein expression in patient cells. GAL-associated disorders include, but are not limited to lysosomal storage diseases, e.g., Fabry disease.

Fabry disease: As used herein, the term “Fabry disease,” also known as alpha-galactosidase A deficiency, Anderson-Fabry disease, angiokeratoma corporis diffusum, angiokeratoma diffuse, and GAL deficiency, refers to a rare inherited disorder of glycosphingolipid metabolism resulting from the absent or markedly deficient activity of the lysosomal enzyme, alpha-galactosidase A (GAL). This enzyme is necessary for the daily metabolism of glycosphingolipids in the body such as globotriaosylceramide (also known as GL-3 or GB-3) and related glycosphingolipids. When proper metabolism of this lipid and other similar lipids does not occur, GL-3 accumulates in the majority of cells throughout the body. The resulting progressive lipid accumulation leads to cell damage. The cell damage causes a wide range of mild to severe symptoms including potentially life-threatening consequences such as kidney failure, heart failure and strokes often at a relatively early age. There are generally two types of Fabry disease which reflects a person's age when symptoms first appear. For the classic type, symptoms appear during childhood or the teenage years. One hallmark disease symptom, a painful burning sensation in the hands and feet, may be noticeable as early as age two. Symptoms get progressively worse over time. For late-onset/atypical type, people don't have symptoms until they're in their 30s or older. The first indication of a problem may be kidney failure or heart disease.

Helper functions: As used herein, the term “helper functions”, as used herein, refers to genes encoding polypeptides which perform functions upon which AAV is dependent for replication (i.e. “helper functions”). The helper functions include those functions required for AAV replication including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus, baculovirus, and vaccinia virus. Helper functions include, without limitation, adenovirus E1, E2a, VA, and E4 or herpesvirus UL5, UL8, UL52, and UL29, and herpesvirus polymerase. In one embodiment, a helper function does not include adenovirus E1.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Lysosomal storage disease: As used herein, the term “lysosomal storage disease” or “lysosomal storage disorder” refers to an inherited metabolic disease that is characterized by an abnormal build-up of various toxic materials in the body's cells as a result of enzyme deficiencies. Lysosomal storage diseases affect different parts of the body, including the skeleton, brain, skin, heart, and central nervous system. Exemplary lysosomal storage diseases include, but are not limited to, Fabry disease, Pompe disease, Gaucher disease, Tay Sachs disease, Cystinosis, Batten disease, Aspartylglucosaminuria, Sandhoff disease, Metachromatic leukodystrophy, Mucolipidosis, Schindler disease, and Niemann-Pick disease. In each case, lysosomal storage diseases are caused by an inborn error of metabolism that results in the absence or deficiency of an enzyme, leading to the inappropriate storage of material in various cells of the body. Most lysosomal storage disorders are inherited in an autosomal recessive manner.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides. One or more mutations may result in a “mutant,” “derivative,” or “variant,” e.g., of a nucleic acid sequence or polypeptide or protein sequence.

Naturally occurring: As used herein, “naturally occurring” or “wild-type” means existing in nature without artificial aid, or involvement of the hand of man. “Naturally occurring” or “wild-type” may refer to a native form of a biomolecule, sequence, or entity.

Nucleic acid: As used herein, the terms “nucleic acid,” “polynucleotide,” and “oligonucleotide” refer to any nucleic acid polymers composed of either polydeoxyribonucleotides (containing 2-deoxy-D-ribose), or polyribonucleotides (containing D-ribose), or any other type of polynucleotide that is an N glycoside of a purine or pyrimidine base, or modified purine or pyrimidine bases. There is no intended distinction in length between the term “nucleic acid,” “polynucleotide,” and “oligonucleotide,” and these terms will be used interchangeably. These terms refer only to the primary structure of the molecule. Thus, these terms include double- and single-stranded DNA, as well as double- and single-stranded RNA.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” or “payload region” or “transgene” refers to one or more polynucleotides or polynucleotide regions encoded by or within a viral genome or an expression product of such polynucleotide or polynucleotide region, e.g., a transgene, a polynucleotide encoding a polypeptide.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells. The payload construct vector may also comprise a component for viral expression in a viral replication cell.

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and/or xylitol.

Pharmaceutical Composition: As used herein, the term “pharmaceutical composition” or pharmaceutically acceptable composition” comprises AAV polynucleotides, AAV genomes, or AAV particle and one or more pharmaceutically acceptable excipients, solvents, adjuvants, and/or the like.

Polypeptide: As used herein, the term “polypeptide” refers to an organic polymer consisting of a large number of amino-acid residues bonded together in a chain. A monomeric protein molecule is a polypeptide.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Promoter: As used herein, the term “promoter” refers to a nucleic acid site to which a polymerase enzyme will bind to initiate transcription (DNA to RNA) or reverse transcription (RNA to DNA).

Regulatory sequence: As used herein, the term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells, those which are constitutively active, those which are inducible, and those which direct expression of the nucleotide sequence only in certain host cells (e.g., tissue-specific regulatory sequences). The expression vectors of the disclosure can be introduced into host cells to thereby produce proteins or portions thereof, including fusion proteins or portions thereof, encoded by nucleic acids as described herein.

Serotype: As used herein, the term “serotype” refers to distinct variations in a capsid of an AAV based on surface antigens which allow epidemiologic classifications of the AAVs at the sub-species level.

Signal Sequences: As used herein, the phrase “signal sequences” refers to a sequence which can direct the transport or localization of a protein to the endoplasmic reticulum during protein synthesis.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: As used herein, the term “subject” or “patient” refers to any organism to which a composition in accordance with the disclosure may be administered, e.g., for experimental, diagnostic, prophylactic, and/or therapeutic purposes. Similarly, “subject” or “patient” refers to an organism who may seek, who may require, who is receiving, or who will receive treatment or who is under care by a trained professional for a particular disease or condition. Typical subjects include animals (e.g., mammals such as mice, rats, rabbits, non-human primates, and humans). In certain embodiments, a subject or patient may be susceptible to or suspected of having a GAL-associated disorder, e.g., a lysosomal storage disorder, e.g., Fabry disease. In certain embodiments, a subject or patient may be diagnosed with Fabry disease.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Treating: As used herein, the term “treating” refers to partially or completely alleviating, ameliorating, improving, relieving, reversing, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence(s). Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, having a sequence that may be wild-type or modified from wild-type and which sequence may encode full-length or partial sequence of a protein, protein domain, or one or more subunits of GAL protein and variants thereof; a polynucleotide encoding GAL protein and variants thereof, having a sequence that may be wild-type or modified from wild-type; and a transgene encoding GAL protein and variants thereof that may or may not be modified from wild-type sequence.

Viral construct vector: As used herein, a “viral construct vector” is a vector which comprises one or more polynucleotide regions encoding or comprising Rep and or Cap protein. A viral construct vector may also comprise one or more polynucleotide region encoding or comprising components for viral expression in a viral replication cell.

Viral genome: As used herein, a “viral genome” or “vector genome” is a polynucleotide comprising at least one inverted terminal repeat (ITR) and at least one encoded payload. A viral genome encodes at least one copy of the payload.

Wild-type: As used herein, “wild-type” is a native form of a biomolecule, sequence, or entity.

Various additional aspects of the methods of the disclosure are described in further detail in the following subsections.

### II. Compositions of the Disclosure

The present disclosure provides compositions comprising isolated, e.g., recombinant, viral particles, e.g., adeno-associated virus (AAV) particles, comprising a liver tropic capsid protein, e.g., an sL65 capsid protein or an LK03 capsid protein, for delivery of a protein, e.g., a GAL protein, and the use of the compositions for treating a subject having a GAL-associated disease or disorder, e.g., a lysosomal storage disorder, e.g., Fabry disease.

The present disclosure also provides compositions comprising a first nucleic acid encoding an AAV capsid protein, e.g., an sL65 capsid protein, wherein the capsid protein comprises an amino acid sequence of SEQ ID NO: 45, or an amino acid sequence at least 85% identical thereto, and a second nucleic acid comprising a transgene encoding a GAL protein.

AAV viruses belonging to the genus Dependovirus of the Parvoviridae family and, as used herein, include any serotype of the over 100 serotypes of AAV viruses known. In general, serotypes of AAV viruses have genomic sequences with a significant homology at the level of amino acids and nucleic acids, provide an identical series of genetic functions, produce virions that are essentially equivalent in physical and functional terms, and replicate and assemble through practically identical mechanisms.

The AAV genome is approximately 4.7 kilobases long and is composed of single-stranded deoxyribonucleic acid (ssDNA) which may be either positive- or negative-sensed. The genome comprises two open reading frames (ORFs) encoding the proteins responsible for replication (Rep) and the structural protein of the capsid (Cap). The open reading frames are flanked by two inverted terminal repeats (ITRs), which serve as the origin of replication of the viral genome. The rep frame is made of four overlapping genes encoding Rep proteins ((Rep78, Rep68, Rep52, Rep40). The cap frame contains overlapping nucleotide sequences of three capsid proteins: VP1, VP2 and VP3. The Rep proteins are important for replication and packaging, while the capsid proteins are assembled to create the protein shell of the AAV, or AAV capsid. See Carter B, Adeno-associated virus and adeno-associated virus vectors for gene delivery, Lassie D, et ah, Eds., “Gene Therapy: Therapeutic Mechanisms and Strategies” (Marcel Dekker, Inc., New York, NY, US, 2000) and Gao G, et al, J. Virol. 2004; 78(12):6381-6388.

The AAV vector typically requires a co-helper to undergo productive infection in cells. In the absence of such helper functions, the AAV virions essentially enter host cells but do not integrate into the cells' genome.

AAV vectors have been investigated for delivery of gene therapeutics because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector, and (v) the non-integrative nature in a host chromosome thereby reducing potential for long-term genetic alterations. Moreover, infection with AAV vectors has minimal influence on changing the pattern of cellular gene expression (Stilwell and Samulski et al., Biotechniques, 2003, 34, 148, the contents of which are herein incorporated by reference in their entirety).

Typically, AAV vectors for GAL protein delivery may be recombinant viral vectors which are replication defective as they lack sequences encoding functional Rep and Cap proteins within the viral genome. In some cases, the defective AAV vectors may lack most or all coding sequences and essentially only contain one or two AAV ITR sequences and a payload sequence.

In certain embodiments, the isolated, e.g., recombinant AAV particles comprises a capsid protein, e.g., a liver tropic capsid protein, e.g., an sL65 capsid protein, and a nucleic acid comprising a transgene encoding a GAL protein. In some embodiments, the transgene further encodes a signal sequence.

In some embodiments, the AAV particles of the present disclosure may be introduced into a mammalian cell, an insect cell or a bacterial cell.

AAV vectors may be modified to enhance the efficiency of delivery. Such modified AAV vectors of the present disclosure can be packaged efficiently and can be used to successfully infect the target cells at high frequency and with minimal toxicity.

In some embodiments, AAV particles of the present disclosure may be used to deliver GAL protein to a specific organ or tissue, e.g., liver (see, e.g., International Patent Application No. PCT/AU2021/050158; the contents of which are herein incorporated by reference in their entirety).

As used herein, the term “AAV vector” or “AAV particle” comprises a capsid and a viral genome comprising a payload. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, e.g., GAL protein.

It is understood that the compositions described herein may have additional conservative or non-essential amino acid substitutions, which do not have a substantial effect on their functions.

### AAV Serotypes

As used herein, an “AAV serotype” is defined primarily by the AAV capsid. The AAV particles of the present disclosure may comprise or be derived from any natural or recombinant AAV serotype. In particular, the AAV particles may utilize or be based on a serotype or include an amino acid sequence of a liver-tropic AAV capsid, e.g., an sL65 capsid protein, and variants thereof.

AAV particles comprising an sL65 capsid protein were demonstrated to possess several essential attributes for liver-targeted capsids. In particular, AAV particles comprising an sL65 capsid protein have a superior liver transduction and transgene expression in non-human primates. In addition, AAV particles comprising an sL65 capsid protein are shown to have a high liver-specific transduction which reduces safety concern risk caused by transgene expression in off-target tissues. Furthermore, AAV particles comprising an sL65 capsid protein result in a broad and uniform distribution throughout the liver, which makes them desirable for both intracellular and secreted protein-based gene therapies. Lastly, AAV particles comprising an sL65 capsid protein can achieve a high yield production in scalable bioreactors, thus enabling manufacturing of cost-effective products.

In one aspect, the present disclosure provides an isolated, e.g., recombinant, AAV particle comprising a capsid protein and a nucleic acid comprising a transgene encoding a GAL protein described herein. In some embodiments, the capsid protein comprises an AAV capsid protein. In some embodiments, the capsid protein comprises an sL65 VP1 capsid protein, or a functional variant thereof.

In some embodiments, the AAV capsid may comprise a sequence, fragment or variant thereof, as described in International Patent Application No. PCT/AU2021/050158, the contents of which are herein incorporated by reference in their entirety, such as, AAV-C11.11 (aka SEQ ID NO: 12) of PCT/AU2021/050158. The nucleic acid encoding the capsid protein comprises the nucleotide sequence, as described in International Patent Application No. PCT/AU2021/050158, such as, AAV-C11.11 (aka SEQ ID NO: 31).

In some embodiments, the AAV capsid protein may comprise an amino acid sequence, fragment or variant thereof, of SEQ ID NO: 45. In some embodiments, the AAV capsid protein may be encoded by a nucleic acid sequence, fragment or variant thereof, of SEQ ID NO: 46.

In some embodiments the AAV serotype of an AAV particle, e.g., an AAV particle for the vectorized delivery of a GAL protein described herein, is sL65, or a variant thereof. In some embodiments, the AAV particle, e.g., a recombinant AAV particle described herein, comprises an sL65 capsid protein.

In some embodiments, the capsid protein, e.g., an sL65 capsid protein, comprises the amino acid sequence of SEQ ID NO: 45 or an amino acid sequence substantially identical (e.g., having at least 70%, 75%, 80%, 85%, 90%, 92%, 95%, 97%, 98%, or 99% sequence identity) thereto. In some embodiments, the capsid protein comprises an amino acid sequence encoded by the nucleotide sequence of SEQ ID NO: 46 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the capsid protein comprises the nucleotide sequence of SEQ ID NO: 46 or a sequence substantially identical (e.g., having at least 70%, 75%, 80%, 85%, 90%, 92%, 95%, 97%, 98%, or 99% sequence identity) thereto.

In some embodiments, the capsid protein comprises an LK03 capsid protein, or a functional variant thereof. In some embodiments, the AAV capsid may comprise a sequence, fragment or variant thereof, as described in International Patent Application Publication No. WO2013029030A1, the contents of which are herein incorporated by reference in their entirety, such as, SEQ ID NO: 31 of WO2013029030A1. The nucleic acid encoding the capsid protein comprises the nucleotide sequence, as described in International Patent Application No. WO2013029030A1, such as, SEQ ID NO: 4.

### AAV Viral Genome

In some aspects, the recombinant AAV particle of the present disclosure serves as an expression vector comprising a viral genome which encodes a GAL protein. In some embodiments, the viral genome may encode a GAL protein and a signal peptide.

In some embodiments, a recombinant AAV particle, e.g., a recombinant AAV particle for the vectorized delivery of a GAL protein described herein, comprises an AAV viral genome, or an AAV vector comprising the viral genome. In some embodiments, the viral genome further comprises one or more of the following: an inverted terminal repeat (ITR) region, an enhancer (e.g., an ApoE/C1 enhancer), a promoter (e.g., an hA1At promoter), an intron region (e.g., a human beta-globin truncated intron), a Kozak sequence, a nucleic acid encoding a transgene encoding a payload (e.g., a GAL protein described herein), a Woodchuck Hepatitis Virus Posttranscriptional Regulatory Element (WPRE) sequence, a poly A signal region, or a combination thereof.

### Viral Genome Component: Inverted Terminal Repeats (ITRs)

In some embodiments, the viral genome may comprise at least one inverted terminal repeat (ITR) region. The AAV particles of the present disclosure comprise a viral genome with at least one ITR region and a payload region, i.e., a transgene encoding a protein, e.g., a GAL protein. The ITR sequence is positioned either 5′ or 3′ relative to the payload region. In some embodiments, the viral genome has two ITRs. These two ITRs flank the payload region at the 5′ and 3′ ends. In some embodiments, the ITR functions as an origin of replication comprising a recognition site for replication. In some embodiments, the ITR comprises a sequence region which can be complementary and symmetrically arranged. In some embodiments, the ITR incorporated into a viral genome described herein may be comprised of a naturally occurring polynucleotide sequence or a recombinantly derived polynucleotide sequence.

The ITRs may be derived from the same serotype as the capsid, or a derivative thereof. The ITR may be of a different serotype than the capsid. In some embodiments, the AAV particle has more than one ITR. In a non-limiting example, the AAV particle has a viral genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the viral genome of the AAV particle are AAV2 ITRs.

Independently, each ITR may be about 100 to about 150 nucleotides in length. In some embodiments, the ITR comprises 100-180 nucleotides in length, e.g., about 100-115, about 100-120, about 100-130, about 100-140, about 100-150, about 100-160, about 100-170, about 100-180, about 110-120, about 110-130, about 110-140, about 110-150, about 110-160, about 110-170, about 110-180, about 120-130, about 120-140, about 120-150, about 120-160, about 120-170, about 120-180, about 130-140, about 130-150, about 130-160, about 130-170, about 130-180, about 140-150, about 140-160, about 140-170, about 140-180, about 150-160, about 150-170, about 150-180, about 160-170, about 160-180, or about 170-180 nucleotides in length. Non-limiting examples of ITR length are 120, 130, 140, 141, 142, 145 nucleotides in length.

In some embodiments, the ITR comprises the nucleotide sequence of any one of SEQ ID NOs: 17-18, or a nucleotide sequence substantially identical (e.g., at least 70%, 75%, 80%, 85%, 90%, 95%, 99% or 100% identical) to any of the aforesaid sequences.

### Viral Genome Component: Promoters and Expression Enhancers

In some embodiments, the viral genome comprises at least one element to enhance the transgene target specificity and expression. Non-limiting examples of elements to enhance the transgene target specificity and expression include promoters, endogenous miRNAs, post-transcriptional regulatory elements (PREs), polyadenylation (PolyA) signal sequences, upstream enhancers (USEs), CMV enhancers, and introns. See, e.g., Powell et al. Viral Expression Cassette Elements to Enhance Transgene Target Specificity and Expression in Gene Therapy, 2015; the contents of which are herein incorporated by reference in their entirety.

In some embodiments, expression of the polypeptides in a target cell may be driven by a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In some embodiments, the viral genome comprises a promoter that is sufficient for expression, e.g., in a target cell, of a payload (e.g., a GAL protein) encoded by a transgene. In some embodiments, the promoter is deemed to be efficient when it drives expression of the polypeptide(s) encoded in the payload region of the viral genome of the AAV particle.

In some embodiments, the promoter is a promoter deemed to be efficient when it drives expression in the cell or tissue being targeted.

In some embodiments, the promoter drives expression of the GAL protein for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiments, the promoter drives expression of a polypeptide (e.g., a GAL polypeptide) for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

In some embodiments, the viral genome comprises a promoter that results in expression in one or more, e.g., multiple, cells and/or tissues, e.g., a ubiquitous promoter. In some embodiments, a promoter which drives or promotes expression in most mammalian tissues includes, but is not limited to, human elongation factor 1α-subunit (EF1α), cytomegalovirus (CMV) immediate-early enhancer and/or promoter, chicken β-actin (CBA) and its derivative CAG, β glucuronidase (GUSB), and ubiquitin C (UBC). Tissue-specific expression elements can be used to restrict expression to certain cell types such as, but not limited to, liver-specific promoters, CNS-specific promoters, B cell promoters, monocyte promoters, leukocyte promoters, macrophage promoters, pancreatic acinar cell promoters, endothelial cell promoters, lung tissue promoters, astrocyte promoters, or various specific nervous system cell- or tissue-type promoters which can be used to restrict expression to neurons, astrocytes, or oligodendrocytes, for example. Exemplary promoters include, but are not limited to, an EF-1a promoter, a chicken β-actin (CBA) promoter and/or its derivative CAG, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hA1AT, TBG), a skeletal muscle promoter (e.g., desmin, MCK, C512) or a fragment, e.g., a truncation, or a functional variant thereof.

In some embodiments, the promoter is a ubiquitous promoter as described in Yu et al. (Molecular Pain 2011, 7:63), Soderblom et al. (E. Neuro 2015), Gill et al., (Gene Therapy 2001, Vol. 8, 1539-1546), and Husain et al. (Gene Therapy 2009), each of which are incorporated by reference in their entirety.

In some embodiments, the viral genome comprises a liver-specific promoter, e.g., a promoter that results in expression of a payload in a hepatic cell and/or tissue. In some embodiments, the liver-specific promoter is a human alpha-1-antitrypsin (A1AT) promoter. In some embodiments, the promoter comprises the nucleotide sequence of SEQ ID NO: 20, or a nucleotide sequence substantially identical (e.g., at least 70%, 75%, 80%, 85%, 90%, 95%, 99% or 100% identical) to the aforesaid sequence.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800, or 700-800 nucleotides.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 nucleotides.

In some embodiments, the viral genome comprises two promoters. As a non-limiting example, the promoters are an A1AT promoter and a CMV promoter.

In some embodiments, the viral genome comprises an enhancer element. The enhancer element, also referred to herein as an “enhancer,” may be, but is not limited to, a tissue-specific enhancer, e.g., a liver-specific enhancer, e.g., a human apolipoprotein E/C-I (ApoE/C-I) gene locus (or hepatic control region). In some embodiments, the enhancer comprises the nucleotide sequence of SEQ ID NO: 19, or a nucleotide sequence substantially identical (e.g., at least 70%, 75%, 80%, 85%, 90%, 95%, 99% or 100% identical) to the aforesaid sequence. In some embodiments, the enhancer is a CMV enhancer.

In some embodiments, the viral genome comprises an enhancer and/or a promoter. In some embodiments, the enhancer is an ApoE/C-I enhancer. In some embodiments, the promoter is an A1AT promoter. In some embodiments, the viral genome comprises an ApoE/C-I enhancer and a human A1AT promoter.

In some embodiments, the viral genome comprises an engineered promoter. In another embodiments, the viral genome comprises a promoter from a naturally expressed protein.

### Viral Genome Component: Introns

In some embodiments, the viral genome comprises at least one intron or a fragment or derivative thereof. In some embodiments, the at least one intron may enhance expression of a GAL protein as described herein. Non-limiting examples of introns include, human β-globin intron (e.g., 476 bps long internally truncated human β-globin intron 2), MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps), and hybrid adenovirus splice donor/IgG splice acceptor (230 bps).

In some embodiments, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

In some embodiments, the viral genome may comprise a human beta-globin intron or a fragment or variant thereof. In some embodiments, the intron comprises one or more human beta-globin sequences (e.g., including fragments/variants thereof). In some embodiments, the promoter may be a human A1AT promoter.

In some embodiments, the intron comprises the nucleotide sequence of SEQ ID NO: 21, or a nucleotide sequence substantially identical (e.g., at least 70%, 75%, 80%, 85%, 90%, 95%, 99% or 100% identical) to any of the aforesaid sequences.

In some embodiments, the encoded protein(s) may be located downstream of an intron in an expression vector such as, but not limited to, SV40 intron or beta globin intron or others known in the art. Further, the encoded GAL protein may also be located upstream of the polyadenylation sequence in an expression vector. In some embodiments, the encoded proteins may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30, 40, 50, 60, or 70 nucleotides downstream from the promoter comprising an intron (e.g., 3′ relative to the promoter comprising an intron) and/or upstream of the polyadenylation sequence (e.g., 5′ relative to the polyadenylation sequence) in an expression vector. In some embodiments, the encoded GAL protein may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30, 25-30, 30-35, 35-40, 45-50, 50-55, 55-60, 60-65 or 65-70 nucleotides downstream from the intron (e.g., 3′ relative to the intron) and/or upstream of the polyadenylation sequence (e.g., 5′ relative to the polyadenylation sequence) in an expression vector. In some embodiments, the encoded proteins may be located within the first 1%, 2%, 3%, 4%, 5%, 6%, 7%, 8%, 9%, 10%, 15%, 20%, 25%, or more than 25% of the nucleotides downstream from the intron (e.g., 3′ relative to the intron) and/or upstream of the polyadenylation sequence (e.g., 5′ relative to the polyadenylation sequence) in an expression vector. In some embodiments, the encoded proteins may be located within the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% of the sequence downstream from the intron (e.g., 3′ relative to the intron) and/or upstream of the polyadenylation sequence (e.g., 5′ relative to the polyadenylation sequence) in an expression vector.

In certain embodiments, the intron sequence is not an enhancer sequence. In some embodiments, the intron sequence is not a sub-component of a promoter sequence. In some embodiments, the intron sequence is a sub-component of a promoter sequence.

### Viral Genome Component: Untranslated Regions (UTRs)

In some embodiments, a wild type untranslated region (UTR) of a gene is transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the disclosure to enhance expression in hepatic cell lines or liver.

In some embodiments, the viral genome encoding a transgene described herein (e.g., a transgene encoding a GAL protein) comprises a Kozak sequence.

While not wishing to be bound by theory, wild-type 5′ untranslated regions (UTRs) include features that play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’. In some embodiments, an advantageous context for initiation of translation in vertebrate mRNAs is GCCACCatgG (SEQ ID NO: 42) (M. Kozak, 1996, Mammalian Genome 7: 563). In some embodiments, the Kozak sequence comprises the nucleotide sequence of SEQ ID NO: 22, or a nucleotide sequence substantially identical (e.g., at least 70%, 75%, 80%, 85%, 90%, 95%, 99% or 100% identical) to the aforesaid sequence.

While not wishing to be bound by theory, wild-type 3′ UTRs are known to have stretches of adenosines and uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in their entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

In some embodiments, the 3′UTR of the viral genome may include a Woodchuck Hepatitis Virus Posttranscriptional Regulatory Element (WPRE). In some embodiments, the WPRE comprise the nucleotide sequence of SEQ ID NO: 25, or a nucleotide sequence substantially identical (e.g., at least 70%, 75%, 80%, 85%, 90%, 95%, 99% or 100% identical) to the aforesaid sequence. In some embodiments, the WPRE comprises the internally truncated nucleotide sequence W3SL of SEQ ID NO: 26, or a nucleotide sequence substantially identical (e.g., at least 70%, 75%, 80%, 85%, 90%, 95%, 99% or 100% identical) to the aforesaid sequence.

Any UTR from any gene known in the art may be incorporated into the viral genome of the AAV particle. These UTRs, or portions thereof, may be placed in the same orientation as in the gene from which they were selected or they may be altered in orientation or location. In some embodiments, the UTR used in the viral genome of the AAV particle may be inverted, shortened, lengthened, or made with one or more other 5′ UTRs or 3′ UTRs known in the art. As used herein, the term “altered,” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome comprises at least one artificial UTR, which is not a variant of a wild type UTR.

In some embodiments, the viral genome comprises UTRs which have been selected from a family of transcripts whose proteins share a common function, structure, feature, or property.

### Viral Genome Component: Polyadenylation Sequence

In some embodiments, the viral genome of the disclosure comprises at least one polyadenylation (polyA) sequence. The viral genome of the disclosure may comprise a polyadenylation sequence between the 3′ end of the payload coding sequence and the 5′ end of the 3′UTR. In some embodiments, the polyA signal region is positioned 3′ relative to the nucleic acid comprising the transgene encoding the payload, e.g., a GAL protein described herein.

In some embodiments, the polyA signal region comprises a length of about 100-600 nucleotides, e.g., about 100-500 nucleotides, about 100-400 nucleotides, about 100-300 nucleotides, about 100-200 nucleotides, about 200-600 nucleotides, about 200-500 nucleotides, about 200-400 nucleotides, about 200-300 nucleotides, about 300-600 nucleotides, about 300-500 nucleotides, about 300-400 nucleotides, about 400-600 nucleotides, about 400-500 nucleotides, or about 500-600 nucleotides. In some embodiments, the polyA signal region comprises a length of about 100 to 150 nucleotides, e.g., about 127 nucleotides. In some embodiments, the polyA signal region comprises a length of about 450 to 500 nucleotides, e.g., about 477 nucleotides. In some embodiments, the polyA signal region comprises a length of about 520 to about 560 nucleotides, e.g., about 552 nucleotides. In some embodiments, the polyA signal region comprises a length of about 127 nucleotides.

In some embodiments, the viral genome comprises a bovine growth hormone (bGH) polyA sequence. In some embodiments, the polyA sequence comprises the nucleotide sequence of SEQ ID NOs: 23, or a nucleotide sequence substantially identical (e.g., at least 70%, 75%, 80%, 85%, 90%, 95%, 99% or 100% identical) to the aforesaid sequence.

In some embodiments, the viral genome comprises an SV40 polyA sequence. In some embodiments, the polyA sequence comprises the nucleotide sequence of SEQ ID NOs: 24, or a nucleotide sequence substantially identical (e.g., at least 70%, 75%, 80%, 85%, 90%, 95%, 99% or 100% identical) to the aforesaid sequence.

### Viral Genome Component: Filler Sequence

In some embodiments, the viral genome comprises one or more filler sequences. The filler sequence may be a wild-type sequence or an engineered sequence. A filler sequence may be a variant of a wild-type sequence. In some embodiments, a filler sequence is a derivative of human albumin.

In some embodiments, the viral genome comprises one or more filler sequences in order to have the length of the viral genome be the optimal size for packaging. In some embodiments, the viral genome comprises at least one filler sequence in order to have the length of the viral genome be about 2.3 kb. In some embodiments, the viral genome comprises at least one filler sequence in order to have the length of the viral genome be about 4.6 kb.

In some embodiments, the viral genome comprises a single stranded (ss) viral genome and comprises one or more filler sequences that, independently or together, have a length about between 0.1 kb-3.8 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, or 3.8 kb. In some embodiments, the total length filler sequence in the vector genome is 3.1 kb. In some embodiments, the total length filler sequence in the vector genome is 2.7 kb. In some embodiments, the total length filler sequence in the vector genome is 0.8 kb. In some embodiments, the total length filler sequence in the vector genome is 0.4 kb. In some embodiments, the length of each filler sequence in the vector genome is 0.8 kb. In some embodiments, the length of each filler sequence in the vector genome is 0.4 kb.

In some embodiments, the viral genome comprises a self-complementary (sc) viral genome and comprises one or more filler sequences that, independently or together, have a length about between 0.1 kb-1.5 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, or 1.5 kb. In some embodiments, the total length filler sequence in the vector genome is 0.8 kb. In some embodiments, the total length filler sequence in the vector genome is 0.4 kb. In some embodiments, the length of each filler sequence in the vector genome is 0.8 kb. In some embodiments, the length of each filler sequence in the vector genome is 0.4 kb.

In some embodiments, the viral genome comprises any portion of a filler sequence. The viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of a filler sequence.

In some embodiments, the viral genome comprises at least one filler sequence and the filler sequence is located 3′ to the 5′ ITR sequence. In some embodiments, the viral genome comprises at least one filler sequence and the filler sequence is located 5′ to a promoter sequence. In some embodiments, the viral genome comprises at least one filler sequence and the filler sequence is located 3′ to the polyadenylation signal sequence. In some embodiments, the viral genome comprises at least one filler sequence and the filler sequence is located 5′ to the 3′ ITR sequence. In some embodiments, the viral genome comprises at least one filler sequence, and the filler sequence is located between two intron sequences. In some embodiments, the viral genome comprises at least one filler sequence, and the filler sequence is located within an intron sequence. In some embodiments, the viral genome comprises two filler sequences, and the first filler sequence is located 3′ to the 5′ ITR sequence and the second filler sequence is located 3′ to the polyadenylation signal sequence. In some embodiments, the viral genome comprises two filler sequences, and the first filler sequence is located 5′ to a promoter sequence and the second filler sequence is located 3′ to the polyadenylation signal sequence. In some embodiments, the viral genome comprises two filler sequences, and the first filler sequence is located 3′ to the 5′ ITR sequence and the second filler sequence is located 5′ to the 5′ ITR sequence.

In some embodiments, the viral genome may comprise one or more filler sequences between one of more regions of the viral genome. In some embodiments, the filler region may be located before a region such as, but not limited to, a payload region, an inverted terminal repeat (ITR), a promoter region, an intron region, an enhancer region, a polyadenylation signal sequence region, and/or an exon region. In some embodiments, the filler region may be located after a region such as, but not limited to, a payload region, an inverted terminal repeat (ITR), a promoter region, an intron region, an enhancer region, a polyadenylation signal sequence region, and/or an exon region. In some embodiments, the filler region may be located before and after a region such as, but not limited to, a payload region, an inverted terminal repeat (ITR), a promoter region, an intron region, an enhancer region, a polyadenylation signal sequence region, and/or an exon region.

In some embodiments, the viral genome comprises a filler sequence after the 5′ ITR. In some embodiments, the viral genome comprises a filler sequence after the promoter region. In some embodiments, the viral genome comprises a filler sequence after the payload region. In some embodiments, the viral genome comprises a filler sequence after the intron region. In some embodiments, the viral genome comprises a filler sequence after the enhancer region. In some embodiments, the viral genome comprises a filler sequence after the polyadenylation signal sequence region. In some embodiments, the viral genome comprises a filler sequence after the exon region.

In some embodiments, the viral genome comprises a filler sequence before the promoter region. In some embodiments, the viral genome comprises a filler sequence before the payload region. In some embodiments, the viral genome comprises a filler sequence before the intron region. In some embodiments, the viral genome comprises a filler sequence before the enhancer region. In some embodiments, the viral genome comprises a filler sequence before the polyadenylation signal sequence region. In some embodiments, the viral genome comprises a filler sequence before the exon region. In some embodiments, the viral genome comprises a filler sequence before the 3′ ITR.

In some embodiments, a filler sequence may be located between two regions, such as, but not limited to, the 5′ ITR and the promoter region. In some embodiments, a filler sequence may be located between two regions, such as, but not limited to, the 5′ ITR and the payload region. In some embodiments, a filler sequence may be located between two regions, such as, but not limited to, the 5′ ITR and the intron region. In some embodiments, a filler sequence may be located between two regions, such as, but not limited to, the 5′ ITR and the enhancer region. In some embodiments, a filler sequence may be located between two regions, such as, but not limited to, the 5′ ITR and the polyadenylation signal sequence region.

In some embodiments, a filler sequence may be located between two regions, such as, but not limited to, the promoter region and the payload region. In some embodiments, a filler sequence may be located between two regions, such as, but not limited to, the promoter region and the intron region. In some embodiments, a filler sequence may be located between two regions, such as, but not limited to, the promoter region and the enhancer region. In some embodiments, a filler sequence may be located between two regions, such as, but not limited to, the promoter region and the polyadenylation signal sequence region. In some embodiments, a filler sequence may be located between two regions, such as, but not limited to, the promoter region and the exon region. In some embodiments, a filler sequence may be located between two regions, such as, but not limited to, the promoter region and the 3′ ITR.

In some embodiments, a filler sequence may be located between two regions, such as, but not limited to, the payload region and the intron region. In some embodiments, a filler sequence may be located between two regions, such as, but not limited to, the payload region and the enhancer region. In some embodiments, a filler sequence may be located between two regions, such as, but not limited to, the payload region and the polyadenylation signal sequence region. In some embodiments, a filler sequence may be located between two regions, such as, but not limited to, the payload region and the exon region.

### Viral Genome Component: Payload

In some embodiments, an recombinant AAV particle, e.g., an AAV particle for the vectorized delivery of a GAL protein, comprises a viral genome encoding a payload. In some embodiments, the viral genome comprises a promoter operably linked to a nucleic acid comprising a transgene encoding a payload. In some embodiments, the payload comprises a GAL protein.

In some embodiments, the disclosure herein provides constructs that allow for improved expression and/or activity of GAL protein delivered by gene therapy vectors.

In some embodiments, the disclosure provides constructs that allow for improved biodistribution of GAL protein delivered by gene therapy vectors.

In some embodiments, the disclosure provides constructs that allow for improved sub-cellular distribution or trafficking of GAL protein delivered by gene therapy vectors.

In some embodiments, the disclosure provides constructs that allow for improved trafficking of GAL protein to lysosomal membranes delivered by gene therapy vectors.

In some aspects, the present disclosure relates to a composition comprising an isolated recombinant AAV particle comprising a liver tropic capsid protein, e.g., an sL65 capsid protein, and a nucleic acid sequence comprising a transgene encoding a GAL protein or functional fragment or variants thereof, and methods of administering or delivering the composition in vitro or in vivo in a subject, e.g., a humans and/or an animal model of disease, e.g., a GAL-associated disease, e.g., a lysosomal storage disease, e.g., Fabry disease.

AAV particles of the present disclosure may comprise a nucleic acid sequence encoding at least one “payload.” As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, e.g., GAL protein or fragment or variant thereof. The payload may comprise any nucleic acid known in the art that is useful for the expression (by supplementation of the protein product or gene replacement using a modulatory nucleic acid) of GAL protein in a target cell transduced or contacted with the AAV particle carrying the payload.

Specific features of a transgene encoding GAL for use in an AAV genome as described herein include the use of a wild type GAL-encoding sequence and codon optimized GAL-encoding constructs.

In some embodiments, the transgene encoding the GAL protein is a wild type GAL-encoding sequence and encodes for a wild type GAL protein or a functional variant thereof.

In some embodiments, a functional variant is a variant that retains some or all of the activity of its wild-type counterpart, so as to achieve a desired therapeutic effect. For example, in some embodiments, a functional variant is effective to be used in gene therapy to treat a disorder or condition, for example, a GLA gene product deficiency or a GAL-associated disorder, e.g., a lysosomal storage disorder, e.g., Fabry disease. Unless indicated otherwise, a variant of a GAL protein as described herein (e.g., in the context of the constructs, vectors, genomes, methods, kits, compositions, etc. of the disclosure) is a functional variant. In some embodiments, the GAL protein comprises amino acids 1-429 of a wild type GAL protein (e.g., GAL protein NP_000160.1). In some embodiments, the GAL protein comprises amino acids 32-429 of a wild type GAL protein. In some embodiments, the encoded GAL protein may be derived from any species, such as, but not limited to human, non-human primate, or rodent.

In some embodiments, the viral genome comprises a payload region (or transgene) encoding a human (Homo sapiens) GAL protein, or a variant thereof.

TABLE 1
Exemplary GAL Sequences

| SEQ ID NO: | Type | Species | Description |
|---|---|---|---|
| 29 | Protein | Homo sapiens | GAL protein NP_000160.1 |
| 30 | DNA | Homo sapiens | GLA mRNA transcript variant 1 |
| | | | NM_000169.3 |

In some embodiments, the viral genome comprises a nucleic acid sequence encoding a polypeptide having at least 85%, e.g., 86%, 87%, 88%, 89%, 90%, 91%, 92%, 93%, 94%, 95%, 96%, 97%, 98%, 99%, or 100% sequence identity to a human GAL protein sequence, or a fragment thereof, as provided in Table 1.

In some embodiments, the GAL protein is derived from a GAL protein encoding sequence of a non-human primate, such as the cynomolgus monkey, Macaca fascicularis.

Certain embodiments provide the GAL protein as a humanized version of a Macaca fascicularis sequence.

In some embodiments, the viral genome comprises a payload region encoding a cynomolgus or crab-eating (long-tailed) macaque (Macaca fascicularis) GAL protein, or a variant thereof.

In some embodiments, the viral genome comprises a payload region encoding a rhesus macaque (Macaca mulatta) GAL protein, or a variant thereof.

In some embodiments, the GAL protein encoded by the transgene may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 1.

In some embodiments, the GAL protein may be encoded by a nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 1.

The GAL protein payloads as described herein can encode any GAL protein, or any portion or derivative of a GAL protein, and are not limited to the GAL proteins or protein-encoding sequences provided in Table 1.

In some embodiments, the GAL protein, or functional variant thereof, comprises the amino acid sequence of SEQ ID NO: 1, or an amino acid substantially identical (e.g., at least 70%, 75%, 80%, 85%, 90%, 95%, 99% or 100% identical) to SEQ ID NO:1.

In some embodiments, the transgene encoding GAL comprises the nucleotide sequence of SEQ ID NO: 2, or a nucleotide sequence substantially identical (e.g., at least 70%, 75%, 80%, 85%, 90%, 95%, 99% or 100% identical) to SEQ ID NO:2.

In some embodiments, a codon-optimized and other variants that encode the same or essentially the same GAL protein amino acid sequence (e.g., those having at least about 90% amino acid sequence identity) may also be used.

In some embodiments, the transgene encoding the GAL protein is codon optimized for expression in mammalian cells including human cells, such as the sequence set forth in SEQ ID NOs: 3-5, or a nucleotide sequence substantially identical (e.g., at least 70%, 75%, 80%, 85%, 90%, 95%, 99% or 100% identical) to any of the aforesaid sequences.

The payload construct may comprise a combination of coding and non-coding nucleic acid sequences.

Any segment, fragment, or the entirety of the viral genome and therein, the payload region, may be codon optimized.

In some embodiments, the viral genome encodes one or more payloads. As a non-limiting example, a viral genome encoding one or more payloads may be replicated and packaged into a viral particle. A target cell transduced with a viral particle comprising one or more payloads may express each of the payloads in a single cell.

In some embodiments, the viral genome may encode a coding or non-coding RNA. In certain embodiments, the adeno-associated viral vector particle further comprises at least one cis-element selected from the group consisting of a Kozak sequence, a backbone sequence, and an intron sequence.

In some embodiments, the payload is a polypeptide which may be a peptide or protein. A protein encoded by the payload construct may comprise a secreted protein, an intracellular protein, an extracellular protein, and/or a membrane protein. The encoded proteins may be structural or functional. Proteins encoded by the viral genome include, but are not limited to, mammalian proteins. In certain embodiments, the AAV particle contains a viral genome that encodes GAL protein or a fragment or variant thereof. The AAV particles described herein may be useful in the fields of human disease, veterinary applications, and a variety of in vivo and in vitro settings.

In some embodiments, a payload may comprise polypeptides that serve as marker proteins to assess cell transformation and expression, fusion proteins, polypeptides having a desired biological activity, gene products that can complement a genetic defect, RNA molecules, transcription factors, and other gene products that are of interest in regulation and/or expression. In some embodiments, a payload may comprise nucleotide sequences that provide a desired effect or regulatory function (e.g., transposons, transcription factors).

The encoded payload may comprise a gene therapy product. A gene therapy product may include, but is not limited to, a polypeptide, RNA molecule, or other gene product that, when expressed in a target cell, provides a desired therapeutic effect. In some embodiments, a gene therapy product may comprise a substitute for a non-functional gene or a gene that is absent, expressed in insufficient amounts, or mutated. In some embodiments, a gene therapy product may comprise a substitute for a non-functional protein or polypeptide or a protein or polypeptide that is absent, expressed in insufficient amounts, misfolded, degraded too rapidly, or mutated. For example, a gene therapy product may comprise a GAL protein or a polynucleotide encoding GAL protein to treat GAL deficiency or GAL-associated disorders.

In some embodiments, the payload encodes a messenger RNA (mRNA). As used herein, the term “messenger RNA” (mRNA) refers to any polynucleotide that encodes a polypeptide of interest and that is capable of being translated to produce the encoded polypeptide of interest in vitro, in vivo, in situ, or ex vivo. Certain embodiments provide the mRNA as encoding GAL or a variant thereof.

The components of an mRNA include, but are not limited to, a coding region, a 5′-UTR (untranslated region), a 3′-UTR, a 5′-cap and a poly-A tail. In some embodiments, the encoded mRNA or any portion of the AAV genome may be codon optimized.

In some embodiments, the protein or polypeptide encoded by the payload construct encoding GAL or a variant thereof is between about 50 and about 4500 amino acid residues in length (hereinafter in this context, “X amino acids in length” refers to X amino acid residues). In some embodiments, the protein or polypeptide encoded is between 50-2000 amino acids in length. In some embodiments, the protein or polypeptide encoded is between 50-1000 amino acids in length. In some embodiments, the protein or polypeptide encoded is between 50-1500 amino acids in length. In some embodiments, the protein or polypeptide encoded is between 50-1000 amino acids in length. In some embodiments, the protein or polypeptide encoded is between 50-800 amino acids in length. In some embodiments, the protein or polypeptide encoded is between 50-600 amino acids in length. In some embodiments, the protein or polypeptide encoded is between 50-400 amino acids in length. In some embodiments, the protein or polypeptide encoded is between 50-200 amino acids in length. In some embodiments, the protein or polypeptide encoded is between 50-100 amino acids in length.

A payload construct encoding a payload may comprise or encode a selectable marker. A selectable marker may comprise a gene sequence or a protein or polypeptide encoded by a gene sequence expressed in a host cell that allows for the identification, selection, and/or purification of the host cell from a population of cells that may or may not express the selectable marker. In some embodiments, the selectable marker provides resistance to survive a selection process that would otherwise kill the host cell, such as treatment with an antibiotic. In some embodiments, an antibiotic selectable marker may comprise one or more antibiotic resistance factors, including but not limited to neomycin resistance (e.g., neo), hygromycin resistance, kanamycin resistance, and/or puromycin resistance.

In some embodiments, a payload construct encoding a payload may comprise a selectable marker including, but not limited to, β-lactamase, luciferase, 0-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540); the contents of each of which are herein incorporated by reference in their entirety.

In some embodiments, a payload construct encoding a selectable marker may comprise a fluorescent protein. A fluorescent protein as herein described may comprise any fluorescent marker including but not limited to green, yellow, and/or red fluorescent protein (GFP, YFP, and/or RFP). In some embodiments, a payload construct encoding a selectable marker may comprise a human influenza hemagglutinin (HA) tag.

In certain embodiments, a nucleic acid for expression of a payload in a target cell will be incorporated into the viral genome and located between two ITR sequences.

### Payload Component: Signal Sequence

In some embodiments, the nucleic acid sequence comprising the transgene encoding the payload, e.g., a GAL protein, comprises a nucleic acid sequence encoding a signal sequence (e.g., a signal sequence region herein). In some embodiments, the nucleic acid sequence comprising the transgene encoding the payload comprises two signal sequence regions. In some embodiments, the nucleic acid sequence comprising the transgene encoding the payload comprises three or more signal sequence regions.

In some embodiments, the nucleotide sequence encoding the signal sequence is located 5′ relative to the nucleotide sequence encoding the GAL protein. In some embodiments, the encoded GAL protein comprises a signal sequence at the N-terminus, wherein the signal sequence is optionally cleaved during cellular processing and/or localization of the GAL protein.

In some embodiments, the signal sequence is a native signal sequence of a GAL protein, e.g., a human GAL protein.

In some embodiments, the human GAL signal sequence may comprise an amino acid sequence of SEQ ID NO: 6, or an amino acid sequence substantially identical (e.g., at least 70%, 75%, 80%, 85%, 90%, 95%, 99% or 100% identical) to SEQ ID NO:6. In some embodiments, the signal sequence may comprise an amino acid sequence having at least one, two, or three, but no more than four modifications, e.g., substitutions, relative to SEQ ID NO: 6.

In some embodiments, the human GAL signal sequence may be encoded by a nucleic acid sequence of SEQ ID NO: 7, or a nucleic acid sequence substantially identical (e.g., at least 70%, 75%, 80%, 85%, 90%, 95%, 99% or 100% identical) to SEQ ID NOs:7.

In some embodiments, the nucleic acid encoding the human GAL signal sequence is codon optimized. In some embodiments, the signal sequence may be encoded by a nucleic acid sequence of any one of SEQ ID NO: 8-10, or a nucleic acid sequence substantially identical (e.g., at least 70%, 75%, 80%, 85%, 90%, 95%, 99% or 100% identical) to any one of SEQ ID NOs:8-10.

In some embodiments, the signal sequence is a heterologous signal sequence.

In some embodiments, the heterologous signal sequence is a human or mouse IgG1 signal sequence.

In some embodiments, the mouse and human IgG1 signal sequence may comprise an amino acid sequence of SEQ ID NO: 11, or an amino acid sequence substantially identical (e.g., at least 70%, 75%, 80%, 85%, 90%, 95%, 99% or 100% identical) to SEQ ID NO: 11. In some embodiments, the signal sequence may comprise an amino acid sequence having at least one, two, or three, but no more than four modifications, e.g., substitutions, relative to SEQ ID NO: 11.

In some embodiments, the human IgG1 signal sequence may be encoded by a nucleic acid sequence of SEQ ID NO: 12, or a nucleic acid sequence substantially identical (e.g., at least 70%, 75%, 80%, 85%, 90%, 95%, 99% or 100% identical) to SEQ ID NO:12.

In some embodiments, the mouse IgG1 signal sequence may be encoded by a nucleic acid sequence of SEQ ID NO: 12, or a nucleic acid sequence substantially identical (e.g., at least 70%, 75%, 80%, 85%, 90%, 95%, 99% or 100% identical) to SEQ ID NO:12.

In some embodiments, the nucleic acid encoding the IgG1 signal sequence (e.g., mouse and/or human sequence) is codon optimized. In some embodiments, the signal sequence may be encoded by a nucleic acid sequence of SEQ ID NO: 13 or 14, or a nucleic acid sequence substantially identical (e.g., at least 70%, 75%, 80%, 85%, 90%, 95%, 99% or 100% identical) to SEQ ID NO:13 or 14.

In some embodiments, the heterologous signal sequence is a synthetic IgG1 signal sequence.

In some embodiments, the synthetic IgG1 signal sequence may comprise an amino acid sequence of SEQ ID NO: 15, or an amino acid sequence substantially identical (e.g., at least 70%, 75%, 80%, 85%, 90%, 95%, 99% or 100% identical) to SEQ ID NO: 15. In some embodiments, the signal sequence may comprise an amino acid sequence having at least one, two, or three, but no more than four modifications, e.g., substitutions, relative to SEQ ID NO: 15.

In some embodiments, the synthetic IgG1 signal sequence may be encoded by a nucleic acid sequence of SEQ ID NO: 16, or a nucleic acid sequence substantially identical (e.g., at least 70%, 75%, 80%, 85%, 90%, 95%, 99% or 100% identical) to SEQ ID NO: 16.

In some embodiments, the encoded signal sequence, e.g., the human GAL signal peptide, comprises the amino acid sequence of SEQ ID NO: 6 or an amino acid sequence substantially identical (e.g., at least 70%, 75%, 80%, 85%, 90%, 95%, 99% or 100% identical) to SEQ ID NO: 6; and the encoded GAL protein comprises the amino acid sequence of SEQ ID NO: 1, or an amino acid sequence substantially identical (e.g., at least 70%, 75%, 80%, 85%, 90%, 95%, 99% or 100% identical) to SEQ ID NO: 1. In some embodiments, the encoded signal sequence is located N-terminal relative to the encoded GAL protein. In some embodiments, the encoded GAL protein having a human GAL signal peptide sequence comprises the amino acid sequence of SEQ ID NO: 27, or an amino acid sequence substantially identical (e.g., at least 70%, 75%, 80%, 85%, 90%, 95%, 99% or 100% identical) to SEQ ID NO: 27.

In some embodiments, the nucleotide sequence encoding the human GAL signal sequence comprises the nucleotide sequence of any one of SEQ ID NOs: 7-10 or a nucleic acid sequence substantially identical (e.g., at least 70%, 75%, 80%, 85%, 90%, 95%, 99% or 100% identical) to any one of SEQ ID NOs: 7-10, and the nucleotide sequence encoding the GAL protein comprises the nucleotide sequence of any one of SEQ ID NOs: 2-5, or a nucleic acid sequence substantially identical (e.g., at least 70%, 75%, 80%, 85%, 90%, 95%, 99% or 100% identical) to any one of SEQ ID NOs: 2-5.

In some embodiments, the nucleotide sequence encoding the signal sequence comprises the nucleotide sequence of SEQ ID NO: 7 or a nucleic acid sequence substantially identical (e.g., at least 70%, 75%, 80%, 85%, 90%, 95%, 99% or 100% identical) to SEQ ID NO: 7, and the nucleotide sequence encoding the GAL protein comprises the nucleotide sequence of SEQ ID NO: 2, or a nucleic acid sequence substantially identical (e.g., at least 70%, 75%, 80%, 85%, 90%, 95%, 99% or 100% identical) to SEQ ID NO: 2.

In some embodiments, the nucleotide sequence encoding the signal sequence comprises the nucleotide sequence of SEQ ID NO: 8 or a nucleic acid sequence substantially identical (e.g., at least 70%, 75%, 80%, 85%, 90%, 95%, 99% or 100% identical) to SEQ ID NO: 8, and the nucleotide sequence encoding the GAL protein comprises the nucleotide sequence of SEQ ID NO: 3, or a nucleic acid sequence substantially identical (e.g., at least 70%, 75%, 80%, 85%, 90%, 95%, 99% or 100% identical) to SEQ ID NO: 3.

In some embodiments, the nucleotide sequence encoding the signal sequence comprises the nucleotide sequence of SEQ ID NO: 9 or a nucleic acid sequence substantially identical (e.g., at least 70%, 75%, 80%, 85%, 90%, 95%, 99% or 100% identical) to SEQ ID NO: 9, and the nucleotide sequence encoding the GAL protein comprises the nucleotide sequence of SEQ ID NO: 4, or a nucleic acid sequence substantially identical (e.g., at least 70%, 75%, 80%, 85%, 90%, 95%, 99% or 100% identical) to SEQ ID NO: 4.

In some embodiments, the nucleotide sequence encoding the signal sequence comprises the nucleotide sequence of SEQ ID NO: 10 or a nucleic acid sequence substantially identical (e.g., at least 70%, 75%, 80%, 85%, 90%, 95%, 99% or 100% identical) to SEQ ID NO: 10, and the nucleotide sequence encoding the GAL protein comprises the nucleotide sequence of SEQ ID NO: 5, or a nucleic acid sequence substantially identical (e.g., at least 70%, 75%, 80%, 85%, 90%, 95%, 99% or 100% identical) to SEQ ID NO: 5.

In some embodiments, the encoded signal sequence, e.g., the human or mouse IgG1 signal peptide, comprises the amino acid sequence of SEQ ID NO: 11 or an amino acid sequence substantially identical (e.g., at least 70%, 75%, 80%, 85%, 90%, 95%, 99% or 100% identical) to SEQ ID NO: 11; and the encoded GAL protein comprises the amino acid sequence of SEQ ID NO: 1, or an amino acid sequence substantially identical (e.g., at least 70%, 75%, 80%, 85%, 90%, 95%, 99% or 100% identical) to SEQ ID NO: 1. In some embodiments, the encoded signal sequence is located N-terminal relative to the encoded GAL protein. In some embodiments, the encoded GAL protein having a human or mouse IgG1 signal peptide sequence comprises the amino acid sequence of SEQ ID NO: 28, or an amino acid sequence substantially identical (e.g., at least 70%, 75%, 80%, 85%, 90%, 95%, 99% or 100% identical) to SEQ ID NO: 28.

In some embodiments, the nucleotide sequence encoding the human or mouse IgG1 signal sequence comprises the nucleotide sequence of any one of SEQ ID NOs: 12-14 or a nucleic acid sequence substantially identical (e.g., at least 70%, 75%, 80%, 85%, 90%, 95%, 99% or 100% identical) to any one of SEQ ID NOs: 12-14, and the nucleotide sequence encoding the GAL protein comprises the nucleotide sequence of any one of SEQ ID NOs: 2-5, or a nucleic acid sequence substantially identical (e.g., at least 70%, 75%, 80%, 85%, 90%, 95%, 99% or 100% identical) to any one of SEQ ID NOs: 2-5.

In some embodiments, the nucleotide sequence encoding the signal sequence comprises the nucleotide sequence of SEQ ID NO: 12 or a nucleic acid sequence substantially identical (e.g., at least 70%, 75%, 80%, 85%, 90%, 95%, 99% or 100% identical) to SEQ ID NO: 12, and the nucleotide sequence encoding the GAL protein comprises the nucleotide sequence of SEQ ID NO: 2, or a nucleic acid sequence substantially identical (e.g., at least 70%, 75%, 80%, 85%, 90%, 95%, 99% or 100% identical) to SEQ ID NO: 2.

In some embodiments, the nucleotide sequence encoding the signal sequence comprises the nucleotide sequence of SEQ ID NO: 13 or a nucleic acid sequence substantially identical (e.g., at least 70%, 75%, 80%, 85%, 90%, 95%, 99% or 100% identical) to SEQ ID NO: 13, and the nucleotide sequence encoding the GAL protein comprises the nucleotide sequence of SEQ ID NO: 3, or a nucleic acid sequence substantially identical (e.g., at least 70%, 75%, 80%, 85%, 90%, 95%, 99% or 100% identical) to SEQ ID NO: 3.

In some embodiments, the nucleotide sequence encoding the signal sequence comprises the nucleotide sequence of SEQ ID NO: 13 or a nucleic acid sequence substantially identical (e.g., at least 70%, 75%, 80%, 85%, 90%, 95%, 99% or 100% identical) to SEQ ID NO: 13, and the nucleotide sequence encoding the GAL protein comprises the nucleotide sequence of SEQ ID NO: 4, or a nucleic acid sequence substantially identical (e.g., at least 70%, 75%, 80%, 85%, 90%, 95%, 99% or 100% identical) to SEQ ID NO: 4.

In some embodiments, the nucleotide sequence encoding the signal sequence comprises the nucleotide sequence of SEQ ID NO: 14 or a nucleic acid sequence substantially identical (e.g., at least 70%, 75%, 80%, 85%, 90%, 95%, 99% or 100% identical) to SEQ ID NO: 14, and the nucleotide sequence encoding the GAL protein comprises the nucleotide sequence of SEQ ID NO: 5, or a nucleic acid sequence substantially identical (e.g., at least 70%, 75%, 80%, 85%, 90%, 95%, 99% or 100% identical) to SEQ ID NO: 5.

### Exemplary Payload: GAL

In some embodiments, an isolated recombinant AAV particle of the disclosure comprises a liver tropic capsid protein, e.g., an sL65 capsid protein, and a nucleic acid comprising a transgene encoding a GAL protein.

In one embodiment, the transgene encoding the GAL protein encodes in 5′ to 3′ order: a signal sequence comprising the amino acid sequence of SEQ ID NO: 6, or an amino acid sequence at least 70%, 75%, 80%, 85%, 90%, 95%, or 99% sequence identity thereto or having at least one, two, or three but no more than four modifications, e.g., substitutions, relative to SEQ ID NO: 6; and a GAL protein comprising the amino acid sequence of SEQ ID NO: 1, or an amino acid sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of any one of SEQ ID NOs: 7-10, or a nucleotide sequence at least 70%, 75%, 80%, 85%, 90%, 95%, or 99% sequence identity thereto; and a nucleotide sequence encoding a GAL protein comprising the nucleotide sequence of any one of SEQ ID NOs: 2-5, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 7, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:2, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 8, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:3, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 9, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:4, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 10, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:5, or a nucleotide sequence at least 70%, 75%, 80%, 85%, 90%, 95%, or 99% sequence identity thereto.

In one embodiment, the transgene encoding the GAL protein encodes in 5′ to 3′ order: a signal sequence comprising the amino acid sequence of SEQ ID NO: 11, or an amino acid sequence at least 70%, 75%, 80%, 85%, 90%, 95%, or 99% sequence identity thereto or having at least one, two, or three but no more than four modifications, e.g., substitutions, relative to SEQ ID NO: 11; and a GAL protein comprising the amino acid sequence of SEQ ID NO: 1, or an amino acid sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of any one of SEQ ID NOs: 12-14, or a nucleotide sequence at least 70%, 75%, 80%, 85%, 90%, 95%, or 99% sequence identity thereto; and a nucleotide sequence encoding a GAL protein comprising the nucleotide sequence of any one of SEQ ID NOs: 2-5, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 12, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:2, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 13, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:3, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 13, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:4, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 14, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:5, or a nucleotide sequence at least 70%, 75%, 80%, 85%, 90%, 95%, or 99% sequence identity thereto.

### Exemplary AAV Viral Genomes and Particles

The AAV particles of the disclosure comprise a capsid protein, e.g., a liver tropic capsid protein, e.g., an sL65 capsid protein, and an AAV viral genome or vector, as described herein.

In some embodiments, the capsid protein comprises an amino acid sequence of SEQ ID NO: 45, or an amino acid sequence substantially identical (e.g., having at least 70%, 75%, 80%, 85%, 90%, 95%, 99% or 100%, sequence identity) thereto. In some embodiments, the capsid protein is encoded by the nucleotide sequence of SEQ ID NO: 46, or a nucleotide sequence substantially identical (e.g., having at least 70%, 75%, 80%, 85%, 90%, 95% or 99%) thereto.

In some embodiments, the viral genome of the AAV particle, described herein, comprises a promoter operably linked to a transgene encoding a GAL protein. In some embodiments, the viral genome further comprises an inverted terminal repeat region, an enhancer, a promoter, an intron, a Kozak sequence, a WPRE sequence, a polyA region, or a combination thereof.

In some embodiments, the viral genome of the AAV particle described herein comprises the nucleotide sequence, e.g., the nucleotide sequence from the 5′ ITR to the 3′ ITR, of the nucleotide sequences of any one of SEQ ID Nos: 31-41 and 51-61, e.g., as described in Table 2, or a nucleotide sequence substantially identical (e.g., having at least about 70%, 75%, 80%, 85%, 90%, 92%, 95%, 97%, 98%, or 99% sequence identity) to any of the aforesaid sequences.

TABLE 2
Exemplary Viral Genome (ITR to ITR) sequences

| Construct | | SEQ ID |
|---|---|---|
| ID | Description (5′ to 3′) | NO: |
| 1 | 5′ITR (SEQ ID NO: 17), Apo E/C-I (SEQ ID NO: 19), A1AT promoter (SEQ | 31, 51 |
| | ID NO: 20), β-globin intron (SEQ ID NO: 21), KOZAK consensus leader | |
| | sequence (SEQ ID NO: 22), hGLA signal peptide (SEQ ID 7), GLA (SEQ ID | |
| | 2), bGH polyA (SEQ ID NO 23), 3′ITR (SEQ ID NO: 18) | |
| 2 | 5′ITR (SEQ ID NO: 17), Apo E/C-I (SEQ ID NO: 19), A1AT (SEQ ID NO: | 32, 52 |
| | 20), β-globin intron (SEQ ID NO: 21), KOZAK consensus leader sequence | |
| | (SEQ ID NO: 22), GLA coA signal peptide (SEQ ID 8), coA GLA (SEQ ID | |
| | 3), bGH polyA (SEQ ID NO 23), 3′ITR (SEQ ID NO: 18) | |
| 3 | 5′ITR (SEQ ID NO: 17), Apo E/C-I (SEQ ID NO: 19), A1AT (SEQ ID NO: | 33, 53 |
| | 20), β-globin intron (SEQ ID NO: 21), KOZAK consensus leader sequence | |
| | (SEQ ID NO: 22), GLA coB signal peptide (SEQ ID 9), coB GLA (SEQ ID | |
| | 4), bGH polyA (SEQ ID NO 23), 3′ITR (SEQ ID NO: 18) | |
| 4 | 5′ITR (SEQ ID NO: 17), Apo E/C-I (SEQ ID NO: 19), A1AT (SEQ ID NO: | 34, 54 |
| | 20), β-globin intron (SEQ ID NO: 21), KOZAK consensus leader sequence | |
| | (SEQ ID NO: 22), GLA coB-CpG signal peptide (SEQ ID 10), coB-CpG | |
| | GLA (SEQ ID 5), bGH polyA (SEQ ID NO 23), 3′ITR (SEQ ID NO: 18) | |
| 5 | 5′ITR (SEQ ID NO: 17), Apo E/C-I (SEQ ID NO: 19), A1AT (SEQ ID NO: | 35, 55 |
| | 20), β-globin intron (SEQ ID NO: 21), KOZAK consensus leader sequence | |
| | (SEQ ID NO: 22), IgG1 signal peptide (SEQ ID 11 and 12), GLA (SEQ ID | |
| | 2), bGH polyA (SEQ ID NO 23), 3′ITR (SEQ ID NO: 18) | |
| 6 | 5′ITR (SEQ ID NO: 17), Apo E/C-I (SEQ ID NO: 19), A1AT (SEQ ID NO: | 36, 56 |
| | 20), β-globin intron (SEQ ID NO: 21), KOZAK consensus leader sequence | |
| | (SEQ ID NO: 22), IgG1 coC signal peptide (SEQ ID 13), coA GLA (SEQ ID | |
| | 3), bGH polyA (SEQ ID NO 23), 3′ITR (SEQ ID NO: 18) | |
| 7 | 5′ITR (SEQ ID NO: 17), Apo E/C-I (SEQ ID NO: 19), A1AT (SEQ ID NO: | 37, 57 |
| | 20), β-globin intron (SEQ ID NO: 21), KOZAK consensus leader sequence | |
| | (SEQ ID NO: 22), IgG1 coC signal peptide (SEQ ID 13), coB GLA (SEQ ID | |
| | 4), bGH polyA (SEQ ID NO 23), 3′ITR (SEQ ID NO: 18) | |
| 8 | 5′ITR (SEQ ID NO: 17), Apo E/C-I (SEQ ID NO: 19), A1AT (SEQ ID NO: | 38, 58 |
| | 20), β-globin intron (SEQ ID NO: 21), KOZAK consensus leader sequence | |
| | (SEQ ID NO: 22), IgG1 coC-CpG signal peptide (SEQ ID 14), coB-CpG | |
| | GLA (SEQ ID 5), bGH polyA (SEQ ID NO 23), 3′ITR (SEQ ID NO: 18) | |
| 9 | 5′ITR (SEQ ID NO: 17), Apo E/C-I (SEQ ID NO: 19), A1AT (SEQ ID NO: | 39, 59 |
| | 20), β-globin intron (SEQ ID NO: 21), KOZAK consensus leader sequence | |
| | (SEQ ID NO: 22), IgG1 coC-CpG signal peptide (SEQ ID 14), coB-CpG | |
| | GLA (SEQ ID 5), WPRE (SEQ ID NO 25), bGH polyA (SEQ ID NO 23), | |
| | 3′ITR (SEQ ID NO: 18) | |
| 10 | 5′ITR (SEQ ID NO: 17), Apo E/C-I (SEQ ID NO: 19), A1AT (SEQ ID NO: | 40, 60 |
| | 20), β-globin intron (SEQ ID NO: 21), KOZAK consensus leader sequence | |
| | (SEQ ID NO: 22), IgG1 coC-CpG signal peptide (SEQ ID 14), coB-CpG | |
| | GLA (SEQ ID 5), WPRE (SEQ ID NO 25), SV polyA (SEQ ID NO 24), | |
| | 3′ITR (SEQ ID NO: 18) | |
| 11 | 5′ITR (SEQ ID NO: 17), Apo E/C-I (SEQ ID NO: 19), A1AT (SEQ ID NO: | 41, 61 |
| | 20), β-globin intron (SEQ ID NO: 21), KOZAK consensus leader sequence | |
| | (SEQ ID NO: 22), IgG1 coC-CpG signal peptide (SEQ ID 14), coB-CpG | |
| | GLA (SEQ ID 5), W3SL (SEQ ID NO 26), SV40 polyA (SEQ ID NO 24), | |
| | 3′ITR (SEQ ID NO: 18) | |

In some embodiments the AAV particle comprises a viral genome comprising the nucleotide sequence of SEQ ID NO: 31 or 51, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 31 or 51 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 7, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 2 or a nucleotide sequence at least 85% (e.g., at least 85, 90, 92, 95, 96, 97, 98, or 99%) identical to the nucleotide sequence of SEQ ID NO: 2; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 31 or 51, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 27, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 32 or 52, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 32 or 52 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 8, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 3 or a nucleotide sequence at least 85% (e.g., at least 85, 90, 92, 95, 96, 97, 98, or 99%) identical to the nucleotide sequence of SEQ ID NO: 3; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 32 or 52, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 27, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 33 or 53, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 33 or 53 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 9, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 4 or a nucleotide sequence at least 85% (e.g., at least 85, 90, 92, 95, 96, 97, 98, or 99%) identical to the nucleotide sequence of SEQ ID NO: 4; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 33 or 53, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 27, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 34 or 54, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 34 or 54 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 10, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 5 or a nucleotide sequence at least 85% (e.g., at least 85, 90, 92, 95, 96, 97, 98, or 99%) identical to the nucleotide sequence of SEQ ID NO: 5; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 34 or 54, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 27, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 35 or 55, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 35 or 55 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 12, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 2 or a nucleotide sequence at least 85% (e.g., at least 85, 90, 92, 95, 96, 97, 98, or 99%) identical to the nucleotide sequence of SEQ ID NO: 2; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 35 or 55, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 28, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 36 or 56, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 36 or 56 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 13, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 3 or a nucleotide sequence at least 85% (e.g., at least 85, 90, 92, 95, 96, 97, 98, or 99%) identical to the nucleotide sequence of SEQ ID NO: 3; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 36 or 56, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 28, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 37 or 57, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 37 or 57 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 13, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 4 or a nucleotide sequence at least 85% (e.g., at least 85, 90, 92, 95, 96, 97, 98, or 99%) identical to the nucleotide sequence of SEQ ID NO: 4; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 37 or 57, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 28, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 38 or 58, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 38 or 58 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 14, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 5 or a nucleotide sequence at least 85% (e.g., at least 85, 90, 92, 95, 96, 97, 98, or 99%) identical to the nucleotide sequence of SEQ ID NO: 5; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 38 or 58, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 28, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 39 or 59, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 39 or 59 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 14, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 5 or a nucleotide sequence at least 85% (e.g., at least 85, 90, 92, 95, 96, 97, 98, or 99%) identical to the nucleotide sequence of SEQ ID NO: 5; a WPRE sequence comprising the nucleotide sequence of SEQ ID NO: 25, or a nucleotide sequence at least 95% identical thereto; a polyadenylation sequence comprising the nucleotide sequence of SEQ ID NO: 23, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 39 or 59, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 28, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 40 or 60, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 40 or 60 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 14, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 5 or a nucleotide sequence at least 85% (e.g., at least 85, 90, 92, 95, 96, 97, 98, or 99%) identical to the nucleotide sequence of SEQ ID NO: 5; a WPRE sequence comprising the nucleotide sequence of SEQ ID NO: 25, or a nucleotide sequence at least 95% identical thereto; a polyadenylation sequence comprising the nucleotide sequence of SEQ ID NO: 24, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 40 or 60, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 28, or an amino acid sequence substantially identical (e.g., having at least 70%, 75%, 80%, 85%, 90%, 95%, or 99% sequence identity) thereto.

In some embodiments the AAV particle comprises a viral genome comprising the nucleotide sequence of SEQ ID NO: 41 or 61, or a nucleotide sequence substantially identical (e.g., having at least 70%, 75%, 80%, 85%, 90%, 95%, 99% or 100%, sequence identity) thereto. In some embodiments, the viral genome comprising the nucleotide sequence of SEQ ID NO: 41 or 61 or nucleotide sequence substantially identical thereto, comprises in 5′ to 3′ order: a 5′ ITR sequence region comprising the nucleotide sequence of SEQ ID NO: 17, or a nucleotide sequence at least 95% identical thereto; an Apo E/C-I enhancer comprising the nucleotide sequence of SEQ ID NO: 19, or a nucleotide sequence at least 95% identical thereto; an A1AT promoter comprising the nucleotide sequence of SEQ ID NO: 20, or a nucleotide sequence at least 95% identical thereto; an intron comprising the nucleotide sequence of SEQ ID NO: 21, or a nucleotide sequence at least 95% identical thereto; a Kozak sequence comprising the nucleotide sequence of SEQ ID NO: 22, or a nucleotide sequence at least 95% identical thereto; a nucleotide sequence encoding a signal sequence comprising the nucleotide sequence of SEQ ID NO: 14, or a nucleotide sequence at least 85% (e.g., at least 85, 90, 92, 95, 96, 97, 98, or 99%) identical thereto; a nucleotide sequence encoding a GAL protein comprising the nucleotide sequence of SEQ ID NO: 5 or a nucleotide sequence at least 85% (e.g., at least 85, 90, 92, 95, 96, 97, 98, or 99%) identical to the nucleotide sequence of SEQ ID NO: 5; a WPRE truncated sequence, W3SL, comprising the nucleotide sequence of SEQ ID NO: 26, or a nucleotide sequence at least 95% identical thereto; a polyadenylation sequence comprising the nucleotide sequence of SEQ ID NO: 24, or a nucleotide sequence at least 95% identical thereto; and a 3′ ITR sequence region comprising the nucleotide sequence of SEQ ID NO: 18, or a nucleotide sequence at least 95% identical thereto.

In some embodiments, the viral genome comprising the nucleotide sequence of SEQ ID NO: 41 or 61, or a nucleotide sequence substantially identical (e.g., having at least 70%, 75%, 80%, 85%, 90%, 95%, or 99% sequence identity) thereto, encodes a GAL protein comprising the amino acid sequence of SEQ ID NO: 28, or an amino acid sequence substantially identical (e.g., having at least 70%, 75%, 80%, 85%, 90%, 95%, or 99% sequence identity) thereto.

In embodiments, the AAV particles comprise an AAV viral genome comprising the nucleotide sequence of any of the viral genomes described herein, e.g., as described in Table 2, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the AAV particles further comprises a capsid protein, e.g., a structural protein. In some embodiments, the capsid protein comprises a VP1 polypeptide, a VP2 polypeptide, and/or a VP3 polypeptide. In some embodiments, the VP1 polypeptide, the VP2 polypeptide, and/or the VP3 polypeptide are encoded by at least one Cap gene. In some embodiments, the capsid protein is an sL65 capsid protein. In some embodiments, the AAV particles further comprise a Rep protein, e.g., a non-structural protein. In some embodiments, the Rep protein comprises a Rep78 protein, a Rep68, Rep52 protein, and/or a Rep40 protein. In some embodiments, the Rep78 protein, the Rep68 protein, the Rep52 protein, and/or the Rep40 protein are encoded by at least one Rep gene.

The present disclosure provides in some embodiments, vectors, cells, and/or AAV particles comprising any of the above identified viral genomes.

In some embodiments, the AAV vector is a single strand vector (ssAAV).

In some embodiments, the AAV vector is a self-complementary AAV vector (scAAV). See, e.g., U.S. Pat. No. 7,465,583. scAAV vectors contain both DNA strands that anneal together to form double stranded DNA. By skipping second strand synthesis, scAAVs allow for rapid expression in the cell.

Methods for producing and/or modifying AAV vectors are disclosed in the art such as pseudotyped AAV vectors (International Patent Publication Nos. WO200028004; WO200123001; WO2004112727; WO 2005005610 and WO 2005072364, the content of each of which are incorporated herein by reference in their entirety).

Nucleic Acids encoding Viral Capsid and Viral Genome

The present disclosure also provides compositions comprising a nucleic acid encoding an AAV capsid protein and a nucleic acid comprising a transgene encoding a GAL protein, e.g., where the two nucleic acids may be located on different vectors. In some embodiments, the compositions comprise a first nucleic acid encoding an AAV capsid protein, e.g., an sL65 capsid protein, wherein the capsid protein comprises an amino acid sequence of SEQ ID NO: 45, or an amino acid sequence at least 70%, 75%, 80%, 85%, 90%, 95%, or 99% identical thereto, and a second nucleic acid comprising a transgene encoding a GAL protein.

In some embodiments, the first nucleic acid encoding the capsid protein comprises a nucleotide sequence of SEQ ID NO: 46, or a nucleotide sequence at least 85% identical thereto. In some embodiments, the encoded GAL protein comprises the amino acid sequence of SEQ ID NO: 1, or an amino acid sequence at least 70%, 75%, 80%, 85%, 90%, 95%, or 99% identical thereto.

In some embodiments, the transgene encoding the GAL protein comprises the nucleotide sequence of SEQ ID NO: 2, or a nucleotide sequence at least 70%, 75%, 80%, 85%, 90%, 95%, or 99% identical thereto.

In some embodiments, the transgene encoding the GAL protein is codon optimized. In some embodiments, the second nucleic acid comprising the transgene encoding the GAL protein comprises the nucleotide sequence of any one of SEQ ID NOs: 3-5, or a nucleotide sequence at least 70%, 75%, 80%, 85%, 90%, 95%, or 99% identical thereto.

In some embodiments, the transgene encoding the GAL protein further encodes a signal sequence. In some embodiments, the encoded signal sequence comprises a human GAL signal peptide. In some embodiments, the encoded signal sequence comprises the amino acid sequence of SEQ ID NO: 6, or an amino acid sequence at least 70%, 75%, 80%, 85%, 90%, 95%, or 99% identical thereto. In some embodiments, the encoded signal sequence comprises an amino acid sequence having at least one, two, or three but no more than four modifications, e.g., substitutions, relative to SEQ ID NO: 6. In some embodiments, the encoded signal sequence is encoded by a nucleic acid comprising the nucleotide sequence of any one of SEQ ID NOs: 7-10, or a nucleotide sequence at least 70%, 75%, 80%, 85%, 90%, 95%, or 99% identical thereto.

In some embodiments, the encoded signal sequence comprises an IgG1 signal peptide (e.g., mouse and/or human peptide). In some embodiments, the encoded signal sequence comprises the amino acid sequence of SEQ ID NO: 11, or an amino acid sequence at least 70%, 75%, 80%, 85%, 90%, 95%, or 99% identical thereto. In some embodiments, the encoded signal sequence comprises the amino acid sequence of SEQ ID NO: 11, or an amino acid sequence having at least one, two, or three but no more than four modifications, e.g., substitutions, relative to SEQ ID NO: 11. In some embodiments, the encoded signal sequence is encoded by a nucleic acid comprising the nucleotide sequence of any one of SEQ ID NOs: 12-14, or a nucleotide sequence at least 70%, 75%, 80%, 85%, 90%, 95%, or 99% identical thereto.

In one embodiment, the transgene encoding the GAL protein encodes in 5′ to 3′ order: a signal sequence comprising the amino acid sequence of SEQ ID NO: 6, or an amino acid sequence at least 70%, 75%, 80%, 85%, 90%, 95%, or 99% sequence identity thereto or having at least one, two, or three but no more than four modifications, e.g., substitutions, relative to SEQ ID NO: 6; and a GAL protein comprising the amino acid sequence of SEQ ID NO: 1, or an amino acid sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of any one of SEQ ID NOs: 7-10, or a nucleotide sequence at least 70%, 75%, 80%, 85%, 90%, 95%, or 99% sequence identity thereto; and a nucleotide sequence encoding a GAL protein comprising the nucleotide sequence of any one of SEQ ID NOs: 2-5, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 7, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:2, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 8, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:3, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 9, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:4, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 10, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:5, or a nucleotide sequence at least 70%, 75%, 80%, 85%, 90%, 95%, or 99% sequence identity thereto.

In one embodiment, the transgene encoding the GAL protein encodes in 5′ to 3′ order: a signal sequence comprising the amino acid sequence of SEQ ID NO: 11, or an amino acid sequence at least 70%, 75%, 80%, 85%, 90%, 95%, or 99% sequence identity thereto or having at least one, two, or three but no more than four modifications, e.g., substitutions, relative to SEQ ID NO: 11; and a GAL protein comprising the amino acid sequence of SEQ ID NO: 1, or an amino acid sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of any one of SEQ ID NOs: 12-14, or a nucleotide sequence at least 70%, 75%, 80%, 85%, 90%, 95%, or 99% sequence identity thereto; and a nucleotide sequence encoding a GAL protein comprising the nucleotide sequence of any one of SEQ ID NOs: 2-5, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 12, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:2, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 13, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:3, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 13, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:4, or a nucleotide sequence at least 70%, 75%, 80%, 85%, 90%, 95%, or 99% sequence identity thereto.

In one embodiment, the transgene encoding the GAL protein comprises in 5′ to 3′ order: a nucleotide sequence encoding a signal sequence comprising the nucleotide sequence of SEQ ID NO: 14, or a nucleotide sequence at least 70%, 75%, 80%, 85%, 90%, 95%, or 99% sequence identity thereto; and a nucleotide sequence encoding a GAL protein comprising the nucleotide sequence of SEQ ID NO:5, or a nucleotide sequence at least 70%, 75%, 80%, 85%, 90%, 95%, or 99% sequence identity thereto.

In some embodiments, the first nucleic acid and/or the second nucleic acid may further comprise one or more of the following: an inverted terminal repeat (ITR) region, a promoter, an enhancer, an intron region, a Kozak sequence, a WPRE sequence, a polyA signal region, or a combination thereof.

In some embodiments, the second nucleic acid comprising the transgene further comprises at least one ITR sequence. The ITR sequence is positioned either 5′ or 3′ relative to the transgene. In some embodiments, the second nucleic acid comprising the transgene comprises two ITRs. These two ITRs flank the transgene at the 5′ and the 3′ ends.

In some embodiments, the second nucleic acid comprising the transgene further comprises a promoter sequence and/or an enhancer. In some embodiments, the promoter is a ubiquitous promoter that results in expression in one or more, e.g., multiple, cells and/or tissues. In some embodiments, the promoter is a tissue-specific promoter, e.g., a promoter that restricts expression to certain cell types, e.g., a liver-specific promoter. In some embodiments, the promoter and/or enhancer is positioned 5′ to the transgene, as described herein. In some embodiments, the promoter and/or enhancer is positioned 5′ to the transgene, as described herein, and at least one ITR sequence is located 5′ to the promoter and/or enhancer.

In some embodiments, the second nucleic acid comprising the transgene further comprises at least one intron or a fragment or derivative thereof. In some embodiments, the at least one intron may enhance the expression of the transgene. In some embodiments, the intron comprises a beta-globin intron or a fragment or variant thereof.

In some embodiments, the second nucleic acid comprising the transgene further comprises a Kozak sequence and/or a WPRE sequence. In some embodiments, the Kozak sequence is positioned 5′ relative to the transgene, as described herein. In some embodiments, the WPRE sequence is positioned 3′ relative to the transgene, as described herein.

In some embodiments, the second nucleic acid comprising the transgene further comprises at least one polyadenylation (polyA) sequence. In some embodiments, the polyA sequence is positioned 3′ relative to the transgene, as described herein. In some embodiments, the polyA sequence is positioned 3′ to the transgene, as described herein, and at least one ITR sequence is located 3′ to the polyA sequence.

In some embodiments, the second nucleic acid comprises, from 5′ to 3′: an ITR sequence, an enhancer, a promoter sequence, an intron, a Kozak sequence, any transgene as described herein, a polyA sequence, and a second ITR sequence.

In some embodiments, the second nucleic acid comprises, from 5′ to 3′: an ITR sequence, an enhancer, a promoter sequence, an intron, a Kozak sequence, any transgene as described herein, a WPRE sequence, a polyA sequence, and a second ITR sequence.

In some embodiments, the first nucleic acid and second nucleic acid are comprised together in a single vector, the vector being comprised in the composition. In some embodiments, the first nucleic acid and the second nucleic acid are comprised in different vectors, wherein both vectors are comprised in the composition.

In some embodiments, the present disclosure provides one or more cells (e.g. a plurality of cells) comprising an isolated rAAV particle as described herein. In some embodiments, the present disclosure provides one or more cells (e.g., a plurality of cells) comprising a nucleic acid composition as described herein.

The present disclosure further provides nucleic acids, e.g., isolated nucleic acids, comprising a transgene encoding an alpha-glucosidase (GAL) protein, wherein the transgene encoding the GAL protein comprises the nucleotide sequence of any one of SEQ ID NOs: 3-5, or a nucleotide sequence at least 70%, 75%, 80%, 85%, 90%, 95%, or 99% identical thereto. The present disclosure also provides compositions comprising a nucleic acid (e.g., isolated nucleic acids) comprising a transgene encoding an alpha-glucosidase (GAL) protein, wherein the transgene encoding the GAL protein comprises the nucleotide sequence of any one of SEQ ID NOs: 3-5, or a nucleotide sequence at least 70%, 75%, 80%, 85%, 90%, 95%, or 99% identical thereto.

In some embodiments, the transgene encoding the GAL protein further encodes a signal sequence. In some embodiments, the encoded signal sequence comprises a human GAL signal peptide. In some embodiments, the encoded signal sequence comprises the amino acid sequence of SEQ ID NO: 6, or an amino acid sequence at least 70%, 75%, 80%, 85%, 90%, 95%, or 99% identical thereto. In some embodiments, the encoded signal sequence comprises an amino acid sequence having at least one, two, or three but no more than four modifications, e.g., substitutions, relative to SEQ ID NO: 6. In some embodiments, the encoded signal sequence is encoded by a nucleic acid comprising a nucleotide sequence of any one of SEQ ID NOs: 7-10, or a nucleotide sequence at least 70%, 75%, 80%, 85%, 90%, 95%, or 99% identical thereto.

In some embodiments, the encoded signal sequence comprises a human IgG1 signal peptide. In some embodiments, the encoded signal sequence comprises the amino acid sequence of SEQ ID NO: 11, or an amino acid sequence at least 70%, 75%, 80%, 85%, 90%, 95%, or 99% identical thereto. In some embodiments, the encoded signal sequence comprises an amino acid sequence having at least one, two, or three but no more than four modifications, e.g., substitutions, relative to SEQ ID NO: 11. In some embodiments, the encoded signal sequence is encoded by a nucleic acid comprising the nucleotide sequence of any one of SEQ ID NOs: 12-14, or a nucleotide sequence at least 70%, 75%, 80%, 85%, 90%, 95%, or 99% identical thereto.

The present disclosure also provides compositions comprising a nucleic acid comprising a transgene encoding a signal sequence. In some embodiments, the encoded signal sequence comprises a human GAL signal peptide. In some embodiments, the encoded signal sequence comprises the amino acid sequence of SEQ ID NO: 6, or an amino acid sequence at least 70%, 75%, 80%, 85%, 90%, 95%, or 99% identical thereto, or an amino acid sequence having at least one, two, or three but no more than four modifications, e.g., substitutions, relative to SEQ ID NO: 6. In some embodiments, the encoded signal sequence is encoded by a nucleic acid comprising a nucleotide sequence of any one of SEQ ID NOs: 8-10, or a nucleotide sequence at least 70%, 75%, 80%, 85%, 90%, 95%, or 99% identical thereto.

The present disclosure also provides compositions comprising a nucleic acid comprising a transgene encoding a signal sequence. In some embodiments, the encoded signal sequence comprises a human IgG1 signal peptide. In some embodiments, the encoded signal sequence comprises the amino acid sequence of SEQ ID NO: 11, or an amino acid sequence at least 70%, 75%, 80%, 85%, 90%, 95%, or 99% identical thereto. In some embodiments, the encoded signal sequence comprises an amino acid sequence having at least one, two, or three but no more than four modifications, e.g., substitutions, relative to SEQ ID NO: 11. In some embodiments, the encoded signal sequence is encoded by a nucleic acid comprising a nucleotide sequence of any one of SEQ ID NOs: 12-14, or a nucleotide sequence at least 70%, 75%, 80%, 85%, 90%, 95%, or 99% identical thereto.

The present disclosure further provides, in some embodiments, an isolated, e.g., recombinant, viral genome (e.g., AAV viral genome) comprising or consisting of the nucleic acid sequence of any one of SEQ ID NO: 31-41 and 51-61. In some embodiments, the viral genome (e.g., AAV viral genome) comprises or consists of the nucleic acid sequence of SEQ ID NO: 31. In some embodiments, the viral genome (e.g., AAV viral genome) comprises or consists of the nucleic acid sequence of SEQ ID NO: 32. In some embodiments, the viral genome (e.g., AAV viral genome) comprises or consists of the nucleic acid sequence of SEQ ID NO: 33. In some embodiments, the viral genome (e.g., AAV viral genome) comprises or consists of the nucleic acid sequence of SEQ ID NO: 34. In some embodiments, the viral genome (e.g., AAV viral genome) comprises or consists of the nucleic acid sequence of SEQ ID NO: 35. In some embodiments, the viral genome (e.g., AAV viral genome) comprises or consists of the nucleic acid sequence of SEQ ID NO: 36. In some embodiments, the viral genome (e.g., AAV viral genome) comprises or consists of the nucleic acid sequence of SEQ ID NO: 37. In some embodiments, the viral genome (e.g., AAV viral genome) comprises or consists of the nucleic acid sequence of SEQ ID NO: 38. In some embodiments, the viral genome (e.g., AAV viral genome) comprises or consists of the nucleic acid sequence of SEQ ID NO: 39. In some embodiments, the viral genome (e.g., AAV viral genome) comprises or consists of the nucleic acid sequence of SEQ ID NO: 40. In some embodiments, the viral genome (e.g., AAV viral genome) comprises or consists of the nucleic acid sequence of SEQ ID NO: 41. In some embodiments, the viral genome (e.g., AAV viral genome) comprises or consists of the nucleic acid sequence of SEQ ID NO: 51. In some embodiments, the viral genome (e.g., AAV viral genome) comprises or consists of the nucleic acid sequence of SEQ ID NO: 52. In some embodiments, the viral genome (e.g., AAV viral genome) comprises or consists of the nucleic acid sequence of SEQ ID NO: 53. In some embodiments, the viral genome (e.g., AAV viral genome) comprises or consists of the nucleic acid sequence of SEQ ID NO: 54. In some embodiments, the viral genome (e.g., AAV viral genome) comprises or consists of the nucleic acid sequence of SEQ ID NO: 55. In some embodiments, the viral genome (e.g., AAV viral genome) comprises or consists of the nucleic acid sequence of SEQ ID NO: 56. In some embodiments, the viral genome (e.g., AAV viral genome) comprises or consists of the nucleic acid sequence of SEQ ID NO: 57. In some embodiments, the viral genome (e.g., AAV viral genome) comprises or consists of the nucleic acid sequence of SEQ ID NO: 58. In some embodiments, the viral genome (e.g., AAV viral genome) comprises or consists of the nucleic acid sequence of SEQ ID NO: 59. In some embodiments, the viral genome (e.g., AAV viral genome) comprises or consists of the nucleic acid sequence of SEQ ID NO: 60. In some embodiments, the viral genome (e.g., AAV viral genome) comprises or consists of the nucleic acid sequence of SEQ ID NO: 61. The present disclosure further provides compositions comprising any of the foregoing viral genomes. The present disclosure further provides cells, e.g., bacterial, mammalian or insect cells, comprising any of the foregoing viral genomes.

### III. Viral Production

### General Viral Production Process

Cells for the production of AAV, e.g., rAAV, particles may comprise, in some embodiments, mammalian cells (such as HEK293 cells) and/or insect cells (such as Sf9 cells).

In various embodiments, AAV production includes processes and methods for producing AAV particles and vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, disclosed herein is a vector comprising a viral genome of the present disclosure. In some embodiments, disclosed herein is a cell comprising a viral genome of the present disclosure. In some embodiments, the cell is a bacterial cell, a mammalian cell (e.g., a HEK293 cell), or an insect cell (e.g., an Sf9 cell).

In some embodiments, disclosed herein is a method of making a recombinant AAV particle of the present disclosure, the method comprising (i) providing a host cell comprising a viral genome described herein, e.g., a nucleic acid comprising a transgene encoding a GAL protein, and incubating the host cell under conditions suitable to enclose the viral genome in a capsid protein, e.g., a capsid protein described herein (e.g., an sL65 capsid protein or functional variant thereof), thereby making the recombinant AAV particle. In some embodiments, the method comprises prior to step (i), introducing a first nucleic acid comprising the viral genome into a cell. In some embodiments, the host cell comprises a second nucleic acid encoding the capsid protein. In some embodiments, the second nucleic acid is introduced into the host cell prior to, concurrently with, or after the first nucleic acid molecule. In some embodiments, the host cell is a bacterial cell, a mammalian cell (e.g., a HEK293 cell), or an insect cell (e.g., an Sf9 cell).

In various embodiments, methods are provided herein of producing AAV particles or vectors by (a) contacting a viral production cell with one or more viral expression constructs encoding at least one AAV capsid protein, and one or more payload constructs encoding a payload molecule, which can be selected from: a transgene, a polynucleotide encoding protein, and a modulatory nucleic acid; (b) culturing the viral production cell under conditions such that at least one AAV particle or vector is produced, and (c) isolating the AAV particle or vector from the production stream.

In these methods, a viral expression construct may encode at least one structural protein and/or at least one non-structural protein. The structural protein may include any of the native or wild type capsid proteins VP1, VP2, and/or VP3, or a chimeric protein thereof. In some embodiments, the VP1 capsid protein may be an sL65 VP1 capsid protein. The non-structural protein may include any of the native or wild type Rep78, Rep68, Rep52, and/or Rep40 proteins or a chimeric protein thereof.

In certain embodiments, contacting occurs via transient transfection, viral transduction, and/or electroporation.

In certain embodiments, the viral production cell is selected from a mammalian cell and an insect cell. In certain embodiments, the insect cell includes a Spodopterafrugiperda insect cell. In certain embodiments, the insect cell includes a Sf9 insect cell. In certain embodiments, the insect cell includes a Sf21 insect cell.

The payload construct vector of the present disclosure may include, in various embodiments, at least one inverted terminal repeat (ITR) and may include mammalian DNA.

Also provided are AAV particles and viral vectors produced according to the methods described herein.

In various embodiments, the AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell or a mammalian cell) with at least one viral expression construct encoding at least one capsid protein and at least one payload construct vector. The viral production cell may be contacted by transient transfection, viral transduction, and/or electroporation. The payload construct vector may include a payload construct encoding a payload molecule such as, but not limited to, a transgene, a polynucleotide encoding protein, and a modulatory nucleic acid. The viral production cell can be cultured under conditions such that at least one AAV particle or vector is produced, isolated (e.g., using temperature-induced lysis, mechanical lysis and/or chemical lysis) and/or purified (e.g., using filtration, chromatography, and/or immunoaffinity purification). As a non-limiting example, the payload construct vector may include mammalian DNA.

In certain embodiments, the AAV particles are produced in an insect cell (e.g., Spodoptera frugiperda (Sf9) cell) using a method described herein. As a non-limiting example, the insect cell is contacted using viral transduction which may include baculoviral transduction.

In certain embodiments, the AAV particles are produced in a mammalian cell (e.g., HEK293 cell) using a method described herein. As a non-limiting example, the mammalian cell is contacted using viral transduction which may include multiplasmid transient transfection (such as triple plasmid transient transfection).

In certain embodiments, the AAV particle production method described herein produces greater than 10¹, greater than 10², greater than 10³, greater than 10⁴, or greater than 10⁵ AAV particles in a viral production cell.

In certain embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

In various embodiments, once administered, an AAV particle disclosed herein may, without being bound by theory, contact a target cell and enter the cell, e.g., in an endosome. The AAV particles, e.g., those released from the endosome, may subsequently contact the nucleus of the target cell to deliver the payload construct. The payload construct, e.g. recombinant viral construct, may be delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

In some embodiments, large scale production of AAV particles utilizes a bioreactor. Without being bound by theory, the use of a bioreactor may allow for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In some embodiments, the bioreactor is used for continuous production in which a portion of the culture is harvested at an experimentally determined time point for purification of AAV particles, and the remaining culture in the bioreactor is refreshed with additional growth media components.

In various embodiments, AAV viral particles can be extracted from viral production cells in a process which includes cell lysis, clarification, sterilization and purification. Cell lysis includes any process that disrupts the structure of the viral production cell, thereby releasing AAV particles. In certain embodiments, cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

In various embodiments, the end result of viral production is a purified collection of AAV particles which include two components: (1) a payload construct (e.g. a recombinant AAV vector genome construct) and (2) a viral capsid.

In certain embodiments, a viral production system or process of the present disclosure includes steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure includes steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BIICs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

### Viral Expression Constructs

In various embodiments, the viral production system of the present disclosure includes one or more viral expression constructs that can be transfected/transduced into a viral production cell. In certain embodiments, a viral expression construct or a payload construct of the present disclosure can be a bacmid, also known as a baculovirus plasmid or recombinant baculovirus genome. In certain embodiments, the viral expression includes a protein-coding nucleotide sequence and at least one expression control sequence for expression in a viral production cell. In certain embodiments, the viral expression includes a protein-coding nucleotide sequence operably linked to least one expression control sequence for expression in a viral production cell. In certain embodiments, the viral expression construct contains parvoviral genes under control of one or more promoters. Parvoviral genes can include nucleotide sequences encoding non-structural AAV replication proteins, such as Rep genes which encode Rep52, Rep40, Rep68, or Rep78 proteins. Parvoviral genes can include nucleotide sequences encoding structural AAV proteins, such as Cap genes which encode VP1, VP2, and VP3 proteins. In some embodiments, the VP1 protein is an sL65 VP1 protein.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of each of which are herein incorporated by reference in their entirety as related to viral expression constructs and uses thereof.

In certain embodiments, the viral expression construct is an AAV expression construct which includes one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may contain a nucleotide sequence which includes start codon region, such as a sequence encoding AAV capsid proteins which include one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG).

In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in their entirety as related to AAV capsid proteins and the production thereof.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG, and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may promote high vector yields.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art.

In certain embodiments, the polynucleotide incorporated into the bacmid (i.e. polynucleotide insert) can include an expression control sequence operably linked to a protein-coding nucleotide sequence. In certain embodiments, the polynucleotide incorporated into the bacmid can include an expression control sequence which includes a promoter, such as p10 or poIh, and which is operably linked to a nucleotide sequence which encodes a structural AAV capsid protein (e.g. VP1, VP2, VP3 or a combination thereof). In some embodiments, the VP1 protein is an sL65 VP1 capsid protein. In certain embodiments, the polynucleotide incorporated into the bacmid can include an expression control sequence which includes a promoter, such as p10 or poIh, and which is operably linked to a nucleotide sequence which encodes a non-structural AAV capsid protein (e.g. Rep78, Rep52, or a combination thereof).

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is: 1:1:10 (VP1:VP2:VP3); 2:2:10 (VP1:VP2:VP3); 2:0:10 (VP1:VP2:VP3); 1-2:0-2:10 (VP1:VP2:VP3); 1-2:1-2:10 (VP1:VP2:VP3); 2-3:0-3:10 (VP1:VP2:VP3); 2-3:2-3:10 (VP1:VP2:VP3); 3:3:10 (VP1:VP2:VP3); 3-5:0-5:10 (VP1:VP2:VP3); or 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the ie-1 promoter, the Δie-1 promoter, has about 20% of the transcriptional activity of that ie-1 promoter. A promoter substantially homologous to the Δie-1 promoter may be used. In respect to promoters, a homology of at least 50%, 60%, 70%, 80%, 90% or more, is considered to be a substantially homologous promoter.

### Mammalian Cells

Viral production of the present disclosure disclosed herein describes processes and methods for producing AAV particles or viral vector that contacts a target cell to deliver a payload construct, e.g. a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO, W138, HeLa, HEK293, HEK293T (293T), Saos, C2C12, L cells, HT1080, Huh7, HepG2, C127, 3T3, CHO, HeLa cells, KB cells, BHK and primary fibroblast, hepatocyte, and myoblast cells derived from mammals. Viral production cells can include cells derived from any mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of each of which are herein incorporated by reference in their entireties insofar as they do no conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the contents of which are herein incorporated by reference in their entirety as related to the 293-10-3 packaging cell line and uses thereof.

In certain embodiments, of the present disclosure a cell line, such as a HeLa cell line, for trans-complementing E1 deleted adenoviral vectors, which encoding adenovirus Ela and adenovirus E1b under the control of a phosphoglycerate kinase (PGK) promoter can be used for AAV particle production as described in U.S. Pat. No. 6,365,394, the contents of which are incorporated herein by reference in their entirety as related to the HeLa cell line and uses thereof.

In certain embodiments, AAV particles are produced in mammalian cells using a multiplasmid transient transfection method (such as triple plasmid transient transfection). In certain embodiments, the multiplasmid transient transfection method includes transfection of the following three different constructs: (i) a payload construct, (ii) a Rep/Cap construct (parvoviral Rep and parvoviral Cap), and (iii) a helper construct. In certain embodiments, the triple transfection method of the three components of AAV particle production may be utilized to produce small lots of virus for assays including transduction efficiency, target tissue (tropism) evaluation, and stability. In certain embodiments, the triple transfection method of the three components of AAV particle production may be utilized to produce large lots of materials for clinical or commercial applications.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other E1a trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference in their entirety as related to the production and processing of recombinant AAV virus particles. Production may also involve methods known in the art including those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g. medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80 C to 37 C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR or ddPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated by reference in their entireties as related to the measurement of particle concentrations).

### Insect Cells

Viral production of the present disclosure includes processes and methods for producing AAV particles or viral vectors that contact a target cell to deliver a payload construct, e.g., a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety as related to the growth and use of insect cells in viral production.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which are herein incorporated by reference in their entirety as related to the use of insect cells in viral production.

In some embodiments, the AAV particles are made using the methods described in WO2015/191508, the contents of which are herein incorporated by reference in their entirety insofar as they do not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the contents of which are herein incorporated by reference in their entirety as related to the production of viral particles.

Expansion, culturing, transfection, infection and storage of insect cells can be carried out in any cell culture media, cell transfection media or storage media known in the art, including Hyclone™ SFX-Insect™ Cell Culture Media, Expression System ESF AF™ Insect Cell Culture Medium, ThermoFisher Sf-900II™ media, ThermoFisher Sf-900III™ media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production Systems

In certain embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can include the viral expression construct (Expression BEV), and one or more group which can include the payload construct (Payload BEV). The baculoviruses may be used to infect a viral production cell for production of AAV particles or viral vector.

In certain embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In certain embodiments, BEVs are produced using a Bacmid Transfection agent, such as Promega FuGENE® HD, WFI water, or ThermoFisher Cellfectin® II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN2 vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety as related to the production and use of BEVs and viral particles.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture. Wasilko D J et al. Protein Expr Purif. 2009 June; 65(2):122-32, the contents of which are herein incorporated by reference in their entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such embodiments, the corresponding bacmid vector is engineered with replication control elements, including but not limited to promoters, enhancers, and/or cell-cycle regulated replication elements.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, the AAV particle of the present disclosure may be produced in insect cells (e.g., Sf9 cells).

In some embodiments, the AAV particle of the present disclosure may be produced using triple transfection.

In some embodiments, the AAV particle of the present disclosure may be produced in mammalian cells.

In some embodiments, the AAV particle of the present disclosure may be produced by triple transfection in mammalian cells.

In some embodiments, the AAV particle of the present disclosure may be produced by triple transfection in HEK293 cells.

The AAV particles comprising the liver tropic capsid protein, e.g., an sL65 capsid protein, and encoding the GAL protein, as described herein, may be useful in the fields of human disease, veterinary applications and a variety of in vivo and in vitro settings. The AAV particles of the present disclosure may be useful in the field of medicine for the treatment, prophylaxis, palliation, or amelioration of GAL-associated diseases and/or disorders, e.g., lysosomal storage diseases, e.g., Fabry disease. In some embodiments, the AAV particles of the disclosure are used for the prevention and/or treatment of GAL-associated disorders, e.g., lysosomal storage diseases, e.g., Fabry disease.

### IV. Pharmaceutical Compositions

The present disclosure additionally provides a method for treating GAL-associated disorders and disorders related to deficiencies in the function or expression of GAL protein(s) in a mammalian subject, including a human subject, comprising administering to the subject a viral particle comprising a liver tropic capsid protein, e.g., an sL65 capsid protein, and a nucleic acid comprising a transgene encoding a GAL protein, or a pharmaceutical composition thereof.

As used herein the term “composition” comprises an AAV particle and at least one excipient. As used herein the term “pharmaceutical composition” comprises an AAV particle and one or more pharmaceutically acceptable excipients.

Although the descriptions of pharmaceutical compositions, e.g., AAV comprising a payload encoding a GAL protein to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients, or subjects.

In some embodiments, the AAV particle formulations described herein may contain a nucleic acid encoding at least one payload. In some embodiments, the formulations may contain a nucleic acid encoding 1, 2, 3, 4, or 5 payloads. In some embodiments, the formulation may contain a nucleic acid encoding a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic proteins, cytoskeletal proteins, intracellular membrane bound proteins, nuclear proteins, proteins associated with human disease, and/or proteins associated with non-human diseases. In some embodiments, the formulation contains at least three payload constructs encoding proteins. Certain embodiments provide that at least one of the payloads is GAL protein or a variant thereof.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In one aspect of the disclosure, an AAV particle of the disclosure will be in the form of a pharmaceutical composition containing a pharmaceutically acceptable carrier. As used herein “pharmaceutically acceptable carrier” refers to any substantially non-toxic carrier conventionally useable for administration of pharmaceuticals in which the isolated polypeptide of the present disclosure will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Suitable pharmaceutically acceptable carriers include any and all solvents, dispersion media, coatings, antibacterial and antifungal agents, isotonic and absorption delaying agents, and the like that are physiologically compatible. Pharmaceutically acceptable carriers also include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the gene therapy vector, use thereof in the pharmaceutical compositions of the disclosure is contemplated. Supplementary active compounds can also be incorporated into the compositions.

Pharmaceutical compositions of the disclosure may be formulated for delivery to animals for veterinary purposes (e.g. livestock (cattle, pigs, dogs, mice, rats), and other non-human mammalian subjects, as well as to human subjects.

In one embodiment, the pharmaceutical compositions of the present disclosure are in the form of injectable compositions. The compositions can be prepared as an injectable, either as liquid solutions or suspensions. The preparation may also be emulsified. Suitable excipients are, for example, water, saline, dextrose, glycerol, ethanol, phosphate buffered saline or the like and combinations thereof. In addition, if desired, the preparation may contain minor amounts of auxiliary substances such as wetting or emulsifying agents, pH-buffering agents, adjuvants, surfactant or immunopotentiators.

Sterile injectable solutions can be prepared by incorporating the compositions of the disclosure in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation include vacuum drying and freeze-drying which yields a powder of the active ingredient plus any additional desired ingredient from a previously sterile-filtered solution thereof.

Toxicity and therapeutic efficacy of nucleic acid molecules described herein can be determined by standard pharmaceutical procedures in cell cultures or experimental animals, e.g., for determining the ED₅₀ (the dose therapeutically effective in 50% of the population). Data obtained from cell culture assays and/or animal studies can be used in formulating a range of dosage for use in humans. The dosage typically will lie within a range of concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays.

### V. Formulations

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

For example, the composition may comprise between 0.1% and 99% (w/w) of the active ingredient. By way of example, the composition may comprise between 0.1% and 100%, e.g., between 0.5% and 50%, between 1-30%, between 5-80%, or at least 80% (w/w) active ingredient.

The AAV particles of the disclosure can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

In some embodiments, the viral vectors encoding GAL protein may be formulated to optimize baricity and/or osmolality. In some embodiments, the baricity and/or osmolality of the formulation may be optimized to ensure optimal drug distribution in the liver.

The formulations of the disclosure can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; the contents of which are herein incorporated by reference in their entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none, or some of the inactive ingredients which may be used in the formulations of the present disclosure may be approved by the US Food and Drug Administration (FDA).

Formulations of AAV particles disclosed herein may include cations or anions. In some embodiments, the formulations include metal cations such as, but not limited to, Zn²⁺, Ca²⁺, Cu²⁺, Mg⁺, or combinations thereof. In some embodiments, formulations may include polymers or polynucleotides complexed with a metal cation (see, e.g., U.S. Pat. Nos. 6,265,389 and 6,555,525, the contents of each of which are herein incorporated by reference in their entirety).

### VI. Methods of the Disclosure

The present disclosure also provides methods of use of the compositions of the disclosure, which generally include administering an AAV particle or a pharmaceutical composition comprising an AAV particle of the disclosure.

In one aspect, the present disclosure provides methods for delivering an exogenous GAL protein to a subject. The methods generally include administering an effective amount of an AAV viral particle or a pharmaceutical composition comprising an AAV particle of the disclosure, thereby delivering the exogenous GAL to the subject.

The present disclosure further provides methods for treating a subject having or diagnosed with having a lsosomal storage disease (e.g., Fabry disease). The methods comprise administering an effective amount of an AAV viral particle or a pharmaceutical composition comprising an AAV particle of the disclosure, thereby treating the lysosomal storage disease in the subject.

The present disclosure also provides methods for treating a subject having or diagnosed with having a GAL-associated disease (e.g., Fabry disease). The methods comprise administering an effective amount of an AAV viral particle or a pharmaceutical composition comprising an AAV particle of the disclosure, thereby treating the GAL-associated disease disease in the subject.

The present disclosure also provides methods for treating a subject having or diagnosed with having Fabry disease. The methods comprise administering an effective amount of an AAV viral particle or a pharmaceutical composition comprising an AAV particle of the disclosure, thereby treating the Fabry disease in the subject.

In some embodiments, AAV particles of the present disclosure, through delivery of a functional payload that is a therapeutic product comprising a GAL protein or variant thereof, can modulate the level or function of a gene product in a subject in need thereof. A functional payload may alleviate or reduce symptoms that result from abnormal level and/or function of a gene product (e.g., an absence or defect in a protein) in a subject in need thereof.

In some embodiments, the delivery of the AAV particles may halt or slow progression of a GAL-associated disorder, e.g., a lysosomal storage disease, e.g., Fabry disease, as measured by the level of GAL in the subject. The level of GAL can be measured using any methods known in the art, for example, by measuring the level of GAL in blood. Males with Fabry disease can usually be diagnosed via an enzyme assay test. Males with classic Fabry disease essentially have no GAL enzyme (less than 1% of normal). Males with a non-classic Fabry gene mutation will have some enzyme but it is still very low. In contrast, Females can have near normal levels of enzyme so an enzyme assay is not sufficient for a diagnosis, therefore, DNA sequence analysis must be performed.

In certain embodiments, the delivery of the AAV particles may improve one or more symptoms of GAL-associated disorders (e.g., Fabry disease), including, for example, numbness, tingling, burning or other abnormal sensations especially in the hands and feet (acroparesthesias), pain attacks/crises, fevers, body ache or discomfort, intolerance to strenuous physical activity, frequent and/or chronic fatigue, hot and cold temperature intolerance, reduced or absent sweating, welling (edema) in the lower legs, ankles and feet, corneal or lenticular opacities—streaked or whorled opaque/cloudy pattern on the cornea and sometimes on the lens of the eye (Corneal verticillata and Fabry cataracts), small, sometimes clustered, slightly raised red or reddish-purple skin lesions (angiokeratoma), gastrointestinal issues, e.g., frequent mild to severe diarrhea and/or constipation, flatulence, stomach or intestinal pain and cramping, early satiety, food intolerance, and difficulty gaining weight, obstructive or constrictive lung disease often evidenced by wheezing, chronic cough, shortness of breath or labored breathing (dyspnea), recurring bronchitis and fatigue (often diagnosed as obstructive pulmonary disease), ringing in the ears (tinnitus), and progressive or sudden hearing loss, weakness, lightheadedness, dizziness, vertigo (spinning dizziness) and headaches from neurological damage, and other cerebrovascular disease impacts, peripheral neuropathy (damage to the peripheral nervous system) which causes or exacerbates many other Fabry disease symptoms, transient ischemic attacks (TIAs), strokes, impaired kidney function and kidney failure often without diabetes, proteinuria and micro-albuminuria, kidney dialysis and transplant, heart complications such as arrhythmias (abnormalities in the heart's rate or rhythm including atrial fibrillation); left ventricular hypertrophy (LVH), and malfunctioning heart valves, heart attacks and heart failure. Improvements in any of these symptoms can be readily assessed according to standard methods and techniques known in the art. Other symptoms not listed above may also be monitored in order to determine the effectiveness of treating Fabry Disease.

In certain embodiments, the subjects in need of treatment are subjects having the classic type Fabry Disease. In other embodiments, the subjects in need of treatment are subjects having late-onset or atypical type Fabry Disease. In certain embodiments, the disclosure provides methods of decreasing glycosphingolipids accumulation, such as in skeletal muscle, cardiac muscle, and/or liver, in any of the foregoing subjects in need by administering an AAV particle or a pharmaceutical composition comprising the AAV particle of the disclosure.

Generally, methods are known in the art for viral infection of the cells of interest. The virus can be placed in contact with the cell of interest or alternatively, can be injected into a subject suffering from a GAL-associated disorder, e.g., a lysosomal storage disease, e.g., Fabry disease.

Guidance in the introduction of the compositions of the disclosure into subjects for therapeutic purposes are known in the art and may be obtained in U.S. Pat. Nos. 5,631,236, 5,688,773, 5,691,177, 5,670,488, 5,529,774, 5,601,818, and PCT Publication No. WO 95/06486, the entire contents of which are incorporated herein by reference.

The AAV particles of the present disclosure may be administered by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), intracranial (into the skull), picutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intraparenchymal (into the substance of), intrathecal (into the spinal canal), intraperitoneal, (infusion or injection into the peritoneum), intravesicular infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, subpial, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In some embodiments, the AAV particles may be delivered by systemic delivery. In some embodiments, the systemic delivery may be by intravascular administration. In some embodiments, the systemic delivery may be by intravenous (IV) administration.

Application of the methods of the disclosure for the treatment and/or prevention of a disorder can result in curing the disorder, decreasing at least one symptom associated with the disorder, either in the long term or short term or simply a transient beneficial effect to the subject.

Accordingly, as used herein, the terms “treat,” “treatment” and “treating” include the application or administration of compositions, as described herein, to a subject who is suffering from a GAL-associated disease, e.g., lysosomal storage disease (e.g., Fabry disease) or who is susceptible to such conditions with the purpose of curing, healing, alleviating, relieving, altering, remedying, ameliorating, improving or affecting such conditions or at least one symptom of such conditions. As used herein, the condition is also “treated” if recurrence of the condition is reduced, slowed, delayed or prevented.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Therapeutically effective amount,” as used herein, is intended to include the amount of a composition of the disclosure that, when administered to a patient for treating a GAL-associated disease, e.g., lysosomal storage disease (e.g., Fabry disease), is sufficient to effect treatment of the disease (e.g., by diminishing, ameliorating or maintaining the existing disease or one or more symptoms of disease). The “therapeutically effective amount” may vary depending on the composition, how the composition is administered, the disease and its severity and the history, age, weight, family history, genetic makeup, stage of pathological processes mediated by the disease expression, the types of preceding or concomitant treatments, if any, and other individual characteristics of the patient to be treated.

“Prophylactically effective amount,” as used herein, is intended to include the amount of a composition that, when administered to a subject who does not yet experience or display symptoms of e.g., a GAL-associated disease, e.g., lysosomal storage disease (e.g., Fabry disease), but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the composition, how the composition is administered, the degree of risk of disease, and the history, age, weight, family history, genetic makeup, the types of preceding or concomitant treatments, if any, and other individual characteristics of the patient to be treated.

A “therapeutically-effective amount” or “prophylacticaly effective amount” also includes an amount of a composition that produces some desired local or systemic effect at a reasonable benefit/risk ratio applicable to any treatment. A composition employed in the methods of the present disclosure may be administered in a sufficient amount to produce a reasonable benefit/risk ratio applicable to such treatment.

The compositions, as described herein, may be administered as necessary to achieve the desired effect and depend on a variety of factors including, but not limited to, the severity of the condition, age and history of the subject and the nature of the composition, for example, the identity of the genes or the affected biochemical pathway.

The pharmaceutical compositions of the disclosure may be administered in a single dose or, in particular embodiments of the disclosure, multiples doses (e.g. two, three, four, or more administrations) may be employed to achieve a therapeutic effect. When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic composition administered in one dose/at one time/single route/single point of contact, i.e., single administration event. In some embodiments, a single unit dose is provided as a discrete dosage form (e.g., a tablet, capsule, patch, loaded syringe, vial, etc.). As used herein, a “total daily dose” is an amount given or prescribed in 24-hour period. It may be administered as a single unit dose. The viral particles may be formulated in buffer only or in a formulation described herein.

The therapeutic or preventative regimens may cover a period of at least about 2, 3, 4, 5, 6, 7, 8, 9, 10, 11, 12, 13, 14, 15, 16, 17, 18, 19, 20, 21, 22, 23 or 24 weeks, or be chronically administered to the subject.

In general, the nucleic acid molecules and/or the vectors of the disclosure are provided in a therapeutically effective amount to elicit the desired effect, e.g., increase GAL expression and/or activity. The quantity of the viral particle to be administered, both according to number of treatments and amount, will also depend on factors such as the clinical status, age, previous treatments, the general health and/or age of the subject, other diseases present, and the severity of the disorder. Precise amounts of active ingredient required to be administered depend on the judgment of the gene therapist and will be particular to each individual patient. Moreover, treatment of a subject with a therapeutically effective amount of the nucleic acid molecules and/or the vectors of the disclosure can include a single treatment or, preferably, can include a series of treatments. It will also be appreciated that the effective dosage used for treatment may increase or decrease over the course of a particular treatment. Changes in dosage may result from the results of diagnostic assays as described herein. The pharmaceutical compositions can be included in a container, pack, or dispenser together with instructions for administration.

In some embodiments, a therapeutically effective amount or a prophylactically effective amount of a viral particle of the disclosure (or pharmaceutical composition of the disclosure) is in titers ranging from about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹², about 1.5×10¹², about 2×10¹², about 2.5×10¹², about 3×10¹², about 3.5×10¹², about 4×10¹², about 4.5×10¹², about 5×10¹², about 5.5×10¹², about 6×10¹², about 6.5×10¹², about 7×10¹², about 7.5×10¹², about 8×10¹², about 8.5×10¹², about 9×10¹², about 9.5×10¹², about 1×10¹³, about 1.5×10¹³, about 2×10³⁴, about 2.5×10¹³, about 3×10¹³, about 3.5×10¹³, about 4×10¹³, about 4.5×10¹³, about 5×10¹³, about 5.5×10¹³, about 6×10¹³, about 6.5×10¹³, about 7×10¹³, about 7.5×10¹³, about 8×10¹³, about 8.5×10¹³, about 9×10¹³, about 9.5×10¹³, about 1×10¹⁴, about 1.5×10¹⁴, about 2×10¹⁴, about 2.5×10¹⁴, about 3×10¹⁴, about 3.5×10¹⁴, about 4×10¹⁴, about 4.5×10¹⁴, about 5×10¹⁴, about 5.5×10¹⁴, about 6×10¹⁴, about 6.5×10¹⁴, about 7×10¹⁴, about 7.5×10¹⁴, about 8×10¹⁴, about 8.5×10¹⁴, about 9×10¹⁴, about 9.5×10¹⁴, about 1×10¹⁵ viral particles (vp).

In some embodiments, a therapeutically effective amount or a prophylactically effective amount of a viral particle of the disclosure (or pharmaceutical composition of the disclosure) is in genome copies (“GC”), also referred to as “viral genomes” (“vg”), ranging from: about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹², about 1.5×10¹², about 2×10¹², about 2.5×10¹², about 3×10¹², about 3.5×10¹², about 4×10¹², about 4.5×10¹², about 5×10¹², about 5.5×10¹², about 6×10¹², about 6.5×10¹², about 7×10¹², about 7.5×10¹², about 8×10¹², about 8.5×10¹², about 9×10¹², about 9.5×10¹², about 1×10¹³, about 1.5×10¹³, about 2×10³⁴, about 2.5×10¹³, about 3×10¹³, about 3.5×10¹³, about 4×10¹³, about 4.5×10¹³, about 5×10¹³, about 5.5×10¹³, about 6×10¹³, about 6.5×10¹³, about 7×10¹³, about 7.5×10¹³, about 8×10¹³, about 8.5×10¹³, about 9×10¹³, about 9.5×10¹³, about 1×10¹⁴, about 1.5×10¹⁴, about 2×10¹⁴, about 2.5×10¹⁴, about 3×10¹⁴, about 3.5×10¹⁴, about 4×10¹⁴, about 4.5×10¹⁴, about 5×10¹⁴, about 5.5×10¹⁴, about 6×10¹⁴, about 6.5×10¹⁴, about 7×10¹⁴, about 7.5×10¹⁴, about 8×10¹⁴, about 8.5×10¹⁴, about 9×10¹⁴, about 9.5×10¹⁴, about 1×10¹⁵, about 1.5×10¹⁵, about 2×10¹⁵, about 2.5×10¹⁵, about 3×10¹⁵, about 3.5×10¹⁵, about 4×10¹⁵, about 4.5×10¹⁵, about 5×10¹⁵, about 5.5×10¹⁵, about 6×10¹⁵, about 6.5×10¹⁵, about 7×10¹⁵, about 7.5×10¹⁵, about 8×10¹⁵, about 8.5×10¹⁵, about 9×10¹⁵, about 9.5×10¹⁵, or about 1×10¹⁶ vg.

Any method known in the art can be used to determine the genome copy (GC) number of the viral compositions of the disclosure. One method for performing AAV GC number titration is as follows: purified AAV viral particle samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR or ddPCR using primer/probe sets targeting specific region of the viral genome.

In certain embodiments of the disclosure, a composition of the disclosure is administered in combination with an additional therapeutic agent or treatment. The compositions and an additional therapeutic agent can be administered in combination in the same composition or the additional therapeutic agent can be administered as part of a separate composition or by another method described herein.

The therapeutic agents may be approved by the US Food and Drug Administration or may be in clinical trial or at the preclinical research stage. The therapeutic agents may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

Examples of additional therapeutic agents or treatments suitable for use in the methods of the disclosure include those agents or treatments known to treat GAL-associated diseases, e.g., Fabry disease. In one embodiment, the additional therapeutic agent or treatment is enzyme replacement therapy. It involves the intravenous administration of recombinant human GAL, e.g., with the product marketed as Fabrazyme® (Genzyme, Inc.) and Replagal® (TKT, Inc.). In other embodiments, the additional therapeutic agents or treatment suitable for use in the methods of the present disclosure include oral chaperone therapy, e.g., Galafold® (or migalastat).

### VII. Kits

The present disclosure also provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the vectors, constructs, or GAL proteins of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

The present disclosure is further illustrated by the following non-limiting examples.

### EXAMPLES

### Example 1. Vector Design and Synthesis

Various AAV viral genomes encoding a human GAL protein were generated. The encoded GAL protein had an amino acid sequence of SEQ ID NO: 1, which corresponded to amino acid 32-429 of human GAL protein (SEQ ID NO: 47), and was encoded by a nucleotide sequence of SEQ ID NO: 2. The nucleic acid sequences encoding the GAL protein were also codon optimized (SEQ ID NOs: 3-5).

The viral genomes encoding the GAL protein were designed to further encode a signal peptide. Some viral genomes were designed to include the native GAL signal peptide having an amino acid sequence of SEQ ID NO:6, which corresponded to amino acid 1-31 of human GAL protein (SEQ ID NO: 47). The native GAL signal peptide was encoded by a nucleotide sequence of SEQ ID NO: 7. The nucleic acid sequences encoding the GAL signal peptide were also codon optimized (SEQ ID NOs: 8-10).

Alternatively, a human IgG1 signal sequence having an amino acid sequence of SEQ ID NO: 11 was incorporated into the viral genomes. The human IgG1 signal peptide was encoded by a nucleotide sequence of SEQ ID NO: 12. The nucleic acid sequences encoding the IgG1 signal peptide were also codon optimized (SEQ ID NOs: 13 and 14).

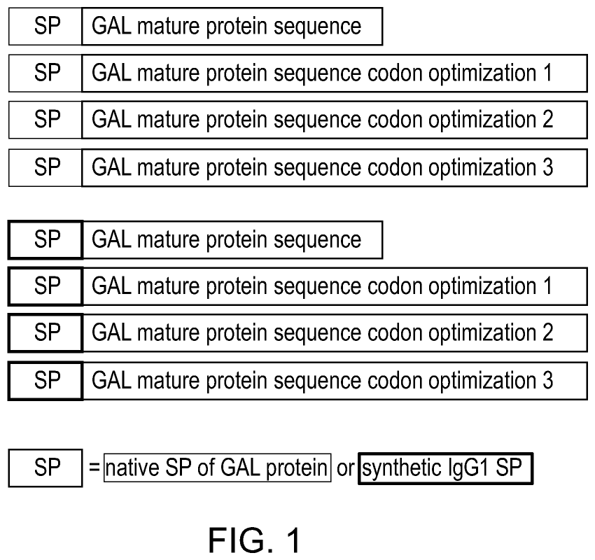

FIG. 1 provides a schematic of exemplary constructs encoding GAL protein having a signal peptide.

### Example 2. Assessment of Expression Constructs In Vitro

Various AAV-GLA constructs combinations were assessed for production of α-GAL mature peptide in vitro.

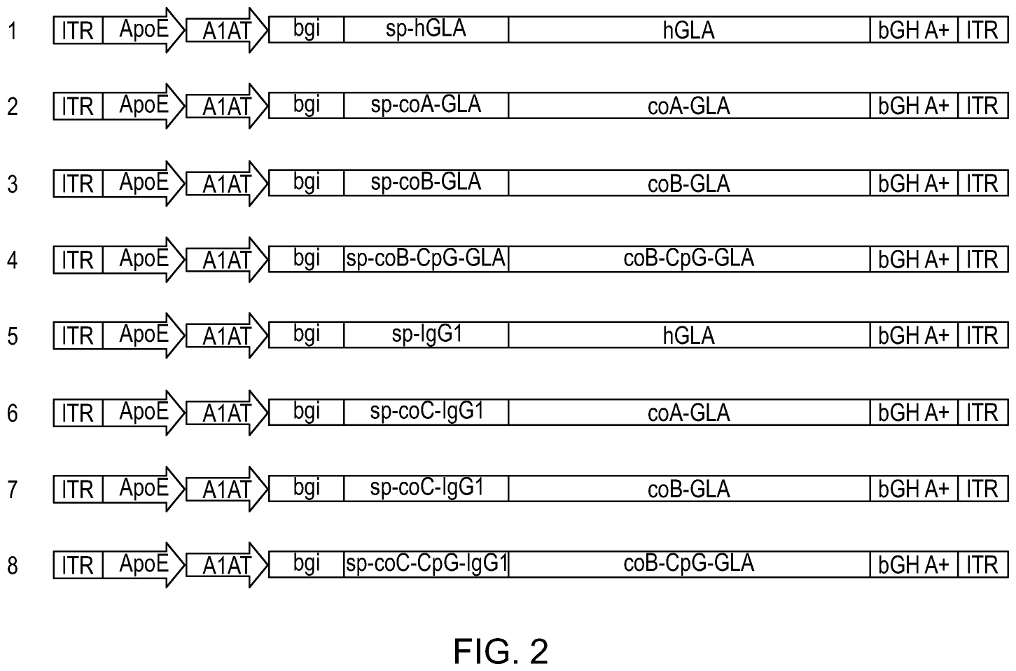

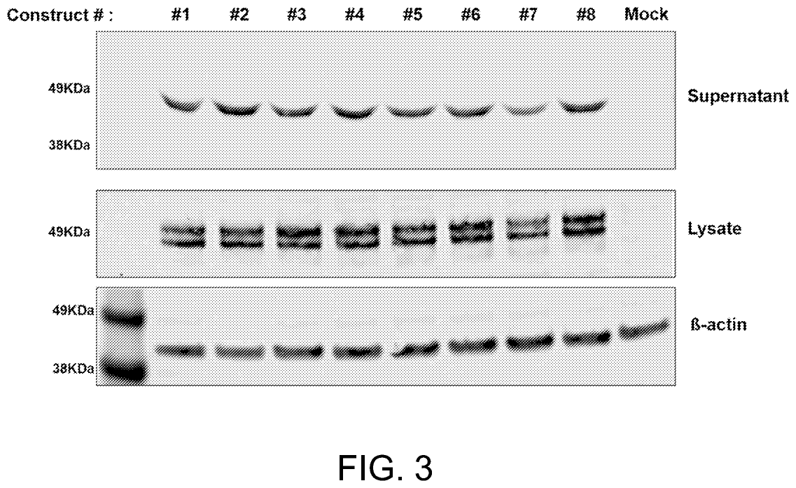

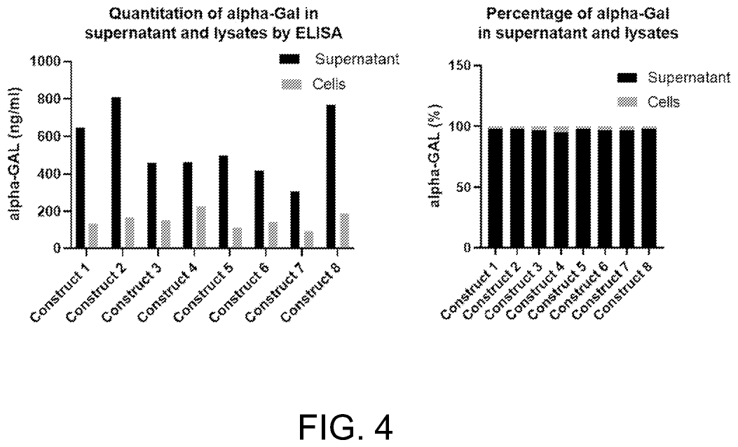

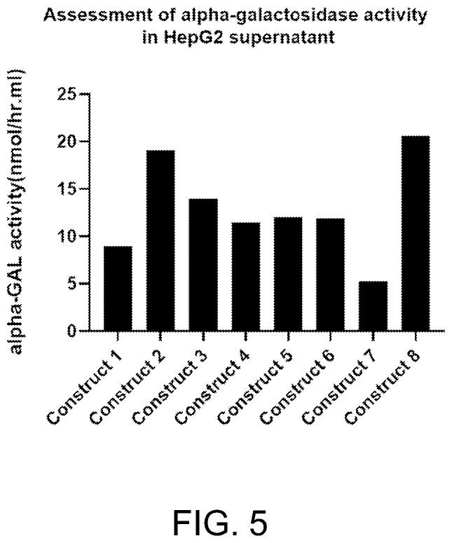

HepG2 cells were transfected with plasmids comprising Constructs 1-8 (FIG. 2). Constructs 1-8 in Examples 2 and 3 (and in the Figures referenced therein) refer to constructs 1A-8A as described herein, or SEQ ID Nos: 31-38, respectively (see Table 6). Cells were harvested 72 hours post-transfection and processed to measure α-GAL levels in both lysate and supernatant. Western blot visualization and ELISA quantification of α-GAL peptide levels demonstrated that certain constructs displayed higher α-GAL expression in both lysate and supernatant, with construct 2 and construct 8 yielding at least 750 ng/ml in supernatants as detected by ELISA (see, FIG. 3, FIG. 4, and Table 3). A β-actin standard was also included as a loading control. Activity of α-GAL protein was assessed for each construct, with construct 2 and construct 8 showing at least 15 nmol/hr*ml in supernatants (see, FIG. 5 and Table 4).

TABLE 3
Fold change in concentration of GAL in
supernatant and lysates vs Construct #1

| Cassettes | Supernatant | Cell lysates |
|---|---|---|
| Construct 1 | 1.00 | 1.00 |
| Construct 2 | 1.25 | 1.29 |
| Construct 3 | 0.71 | 1.15 |
| Construct 4 | 0.72 | 1.74 |
| Construct 5 | 0.77 | 0.85 |
| Construct 6 | 0.64 | 1.09 |
| Construct 7 | 0.47 | 0.70 |
| Construct 8 | 1.19 | 1.46 |

TABLE 4
Fold change in GAL activity in supernatant vs Construct #1

| | Activity | Fold |
|---|---|---|
| Cassettes | (nmol/h · ml) | change |
| Construct 1 | 9.0 | 1.0 |
| Construct 2 | 19.1 | 2.1 |
| Construct 3 | 14.0 | 1.6 |
| Construct 4 | 11.5 | 1.3 |
| Construct 5 | 12.0 | 1.3 |
| Construct 6 | 11.9 | 1.3 |
| Construct 7 | 5.3 | 0.6 |
| Construct 8 | 20.6 | 2.3 |

The present example demonstrates, among other things, that constructs of the present disclosure may produce mature, functional GAL in vitro. In some embodiments, level and/or activity of GAL may be improved relative to a reference construct.

### Example 3. Assessment of Expression Constructs In Vivo

Various AAV-GLA constructs combinations were assessed for production and activity of α-GAL mature peptide in vivo.

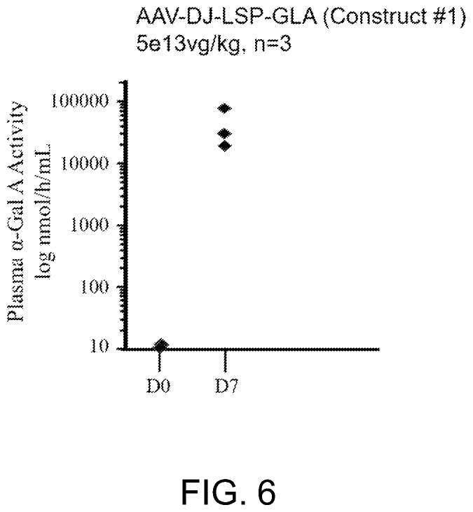

Construct 1 (FIG. 2) was packaged in AAV-DJ and delivered to 14-week-old Fabry mice (n=3) via intravenous (IV) administration. Construct 1 in Examples 3 refers to construct 1A as described herein, or SEQ ID No: 31 (see Table 6). Compositions were administered into the orbital vein at a dose of 5E13 vg/kg. Plasma samples were harvested at day 0 and post-injection day 7 and GAL activity was assessed (FIG. 6).

The present example demonstrates, among other things, that constructs of the present disclosure produce mature, functional GAL in vivo. In some embodiments, level and/or activity of GAL may be improved relative to a reference construct.

The constructs as shown in FIG. 2 were packaged within AAV ITRs under a promoter, and tested for expression. There constructs were also tested for GLA enzymatic activity in cells.

AAV particles comprising the viral genomes are generated. These recombinant AAV particles comprise the liver tropic capsid protein sL65 having an amino acid sequence of SEQ ID NO: 45. The capsid protein is encoded by a nucleic acid having a nucleotide sequence of SEQ ID NO: 46.

### Example 4. Assessment of Expression Constructs In Vitro

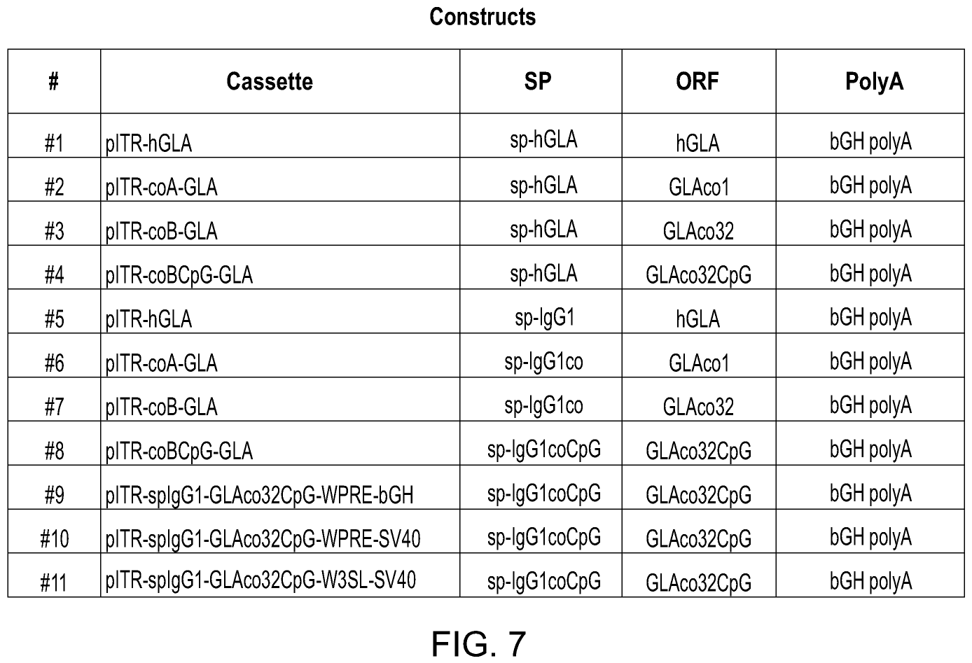

AAV-GLA constructs combinations were assessed for production of α-GAL mature peptide in vitro. HepG2 cells were transfected with plasmids comprising Constructs 1-11 (FIG. 7). Constructs 1-11 in Examples 4 and 5, and the Figures referenced therein, refer to constructs 1B-11B, or SEQ ID Nos: 51-61, respectively (see Table 6). Constructs 1B-11B differ from constructs 1A-11A in that constructs 1B-11B contain a 2 base pair addition (of “gt”) in the filler sequence immediately 5′ to the 3′ ITR, and a 6 base pair deletion outside of, and in the filler sequence immediately 3′ to, the 5′ ITR.

Briefly, cells were seeded in 12-well plates in cell culture media (DMEM and 10% FBS, 2 ml/well). Plasmid and lipid complex were prepared according to manufacturer's instruction. 2 μg DNA and Opti-MEM was mixed in a total volume of 92 μl, and 6 μl Fugene HD transfection reagent (Promega) was added and vortexed immediately for 5 seconds. The DNA/lipid complex was incubated at room temperature for 15 minutes, and then added to the cells for incubation at 37° C. for 48-72 hours.

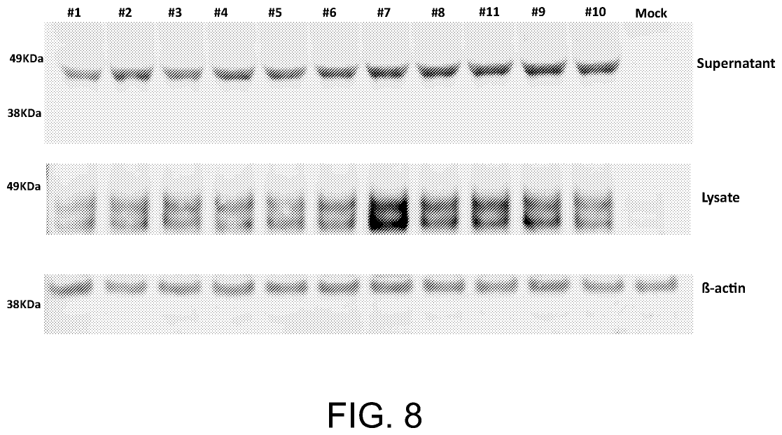

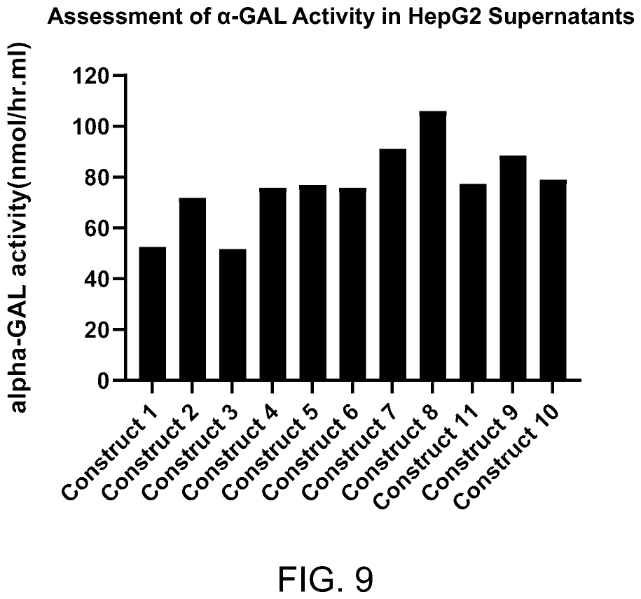

Western blot visualization of α-GAL peptide levels demonstrated that certain constructs displayed higher α-GAL expression in both lysate and supernatant (FIG. 8). A β-actin standard was also included as a loading control. The activity of α-GAL protein was also assessed for each construct, with constructs 2, 4, 5, 6, 7, 8, 9, 10 and 11 showing at least 70 nmol/hr*ml in supernatants (FIG. 9 and Table 5).

TABLE 5
Fold change in α-GAL activity in supernatant vs Construct #1

| | Activity | Fold |
|---|---|---|
| Cassettes | (nmol/h · ml) | change |
| Construct 1 | 52.5 | 1 |
| Construct 2 | 71.9 | 1.4 |
| Construct 3 | 51.7 | 1 |
| Construct 4 | 75.9 | 1.4 |
| Construct 5 | 77.0 | 1.5 |
| Construct 6 | 75.9 | 1.4 |
| Construct 7 | 91.2 | 1.7 |
| Construct 8 | 105.9 | 2 |
| Construct 11 | 77.3 | 1.5 |
| Construct 9 | 88.6 | 1.7 |
| Construct 10 | 79.0 | 1.5 |

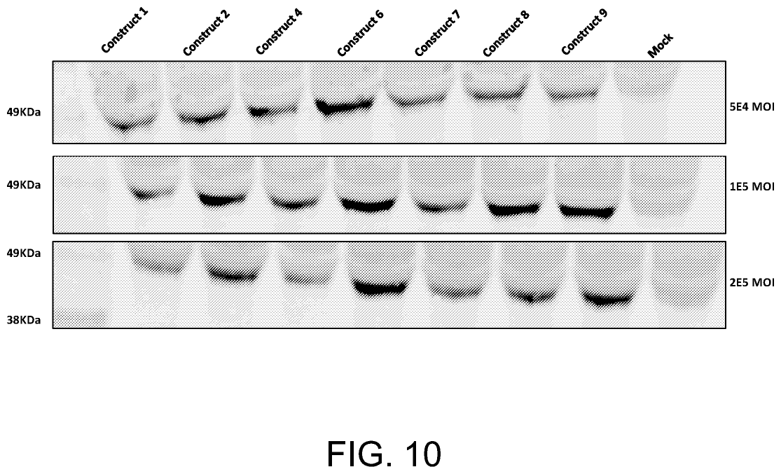

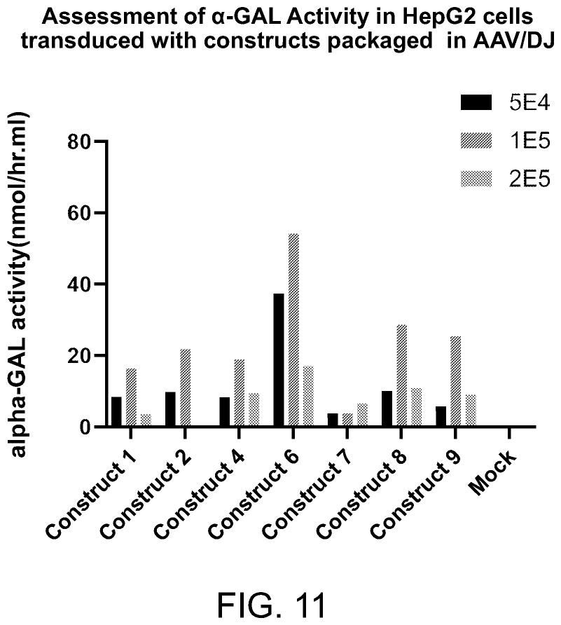

HepG2 cells were also transduced with select constructs (i.e., constructs 2, 4, 6, 7, 8 and 9) packaged into AAV/DJ along with Ref2K. Briefly, cells were plated 500,000 cells in 12 well plate on Day 0, and transduced with constructs packaged into AAV/DJ at three different multiplicity of infection (MOIs), i.e., 5E4, 1E5 and 2E5, on Day 1. Cells were harvested 72 hours post-transduction and supernant samples were collected. Western blot visualization of α-GAL peptide levels demonstrated that transduction at a MOI of 1E5 resulted in a better expression for GAL, and certain constructs displayed higher α-GAL expression in the supernatant, such as constructs 6, 8 and 9 (FIG. 10). Activity of α-GAL protein was also assessed for each construct. Consistent with the Western blot results, transduction at a MOI of 1E5 resulted in a higher GAL activity level, in particular, for constructs 6, 8 and 9 (FIG. 11).

### Example 5. Assessment of Expression Constructs in AAV-DJ In Vivo

AAV-GLA constructs combinations were assessed for production and activity of α-GAL mature peptide in vivo. Constructs 1-11 in Example 5, and the Figures referenced therein, refer to constructs 1B-11B, or SEQ ID Nos: 51-61, respectively (see Table 6).

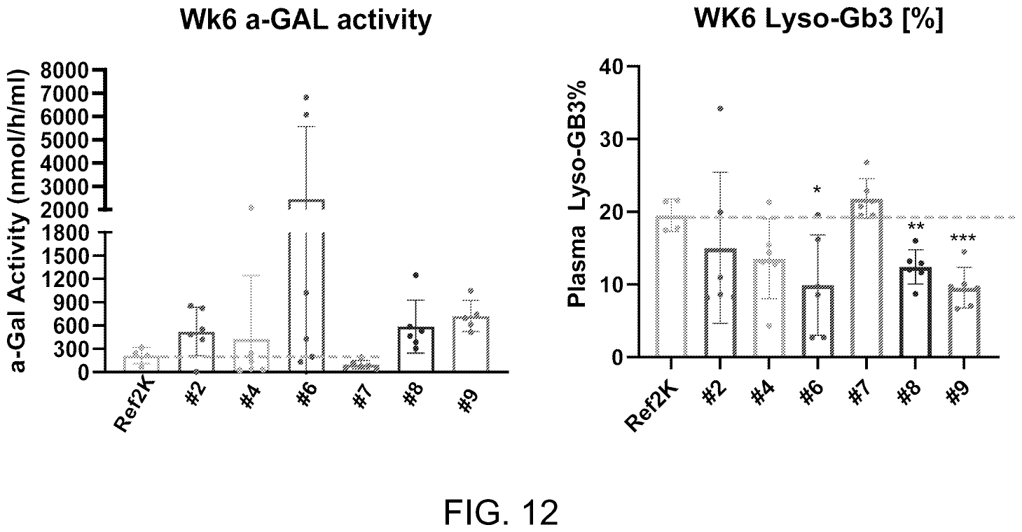

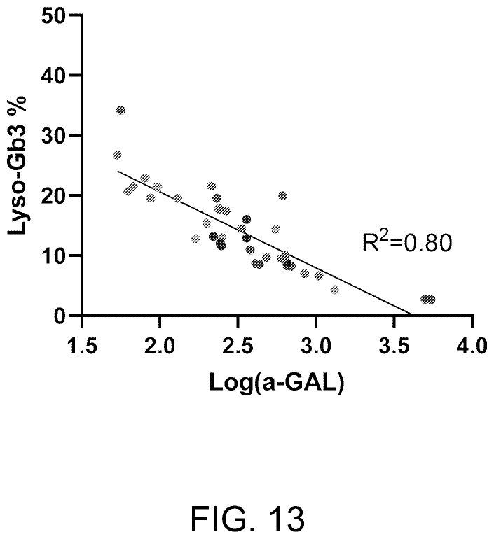

Briefly, Fabry mice (B6; 129-Gla^{tm1Kul}/J; JAX 003535) (n=5 per group) were dosed with codon optimized constructs in AAV-DJ and monitored for 6 weeks. Compositions were administered in formulation buffer (10 mM Sodium Phosphate, 150 mM NaCl and 0.001% Pluronic F68) at a dose of 5E11 vg/kg. The animals were sacrificed after Week 6. α-GAL activity was measured in plasma, and clearance of lyso-GB3 accumulation was also evaluated (FIG. 12). As shown in FIG. 12, mice receiving constructs 6, 8 and 9 had a significant increase in α-GAL activity, as well as a significant reduction in lyso-GB3 levels. The correlation between α-GAL and lyso-GB3 levels is shown in FIG. 13.

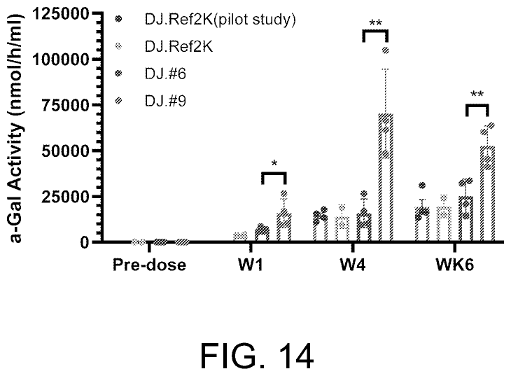

Constructs 6 and 9 in AAV-DJ were tested further in Fabry mice (n=4 per group) at a higher dose of 1E13 vg/kg, as described above. Plasma samples were harvested pre-dose, and at Week 1, Week 4 and Week 6, and α-GAL activity was measured. As shown in FIG. 14, mice receiving construct 9 had the highest level of α-GAL activity.

The present example demonstrates, among other things, that constructs of the present disclosure produce mature, functional GAL in vivo, and the level and/or activity of GAL were significantly improved in mice receiving the constructs of the present disclosure.

### Example 6. Assessment of Expression Constructs Packaged in AAV Particle Containing SL65 Capsid In Vivo

Selected expression constructs were chosen for assessment in AAV particles comprising a liver tropic capsid protein SL65. The liver tropic capsid protein sL65 comprises an amino acid sequence of SEQ ID NO: 45. The capsid protein is encoded by a nucleic acid having a nucleotide sequence of SEQ ID NO: 46. The production and activity of α-GAL mature peptide was evaluated in vivo.

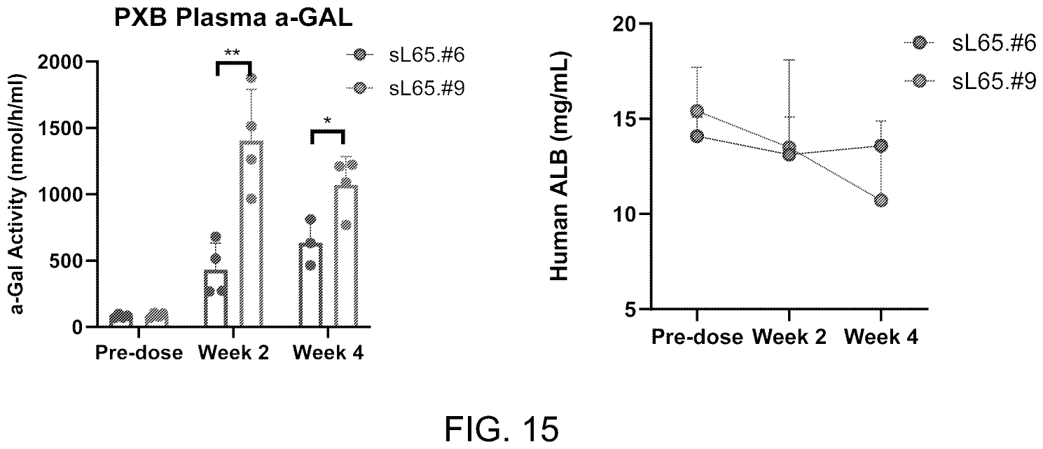

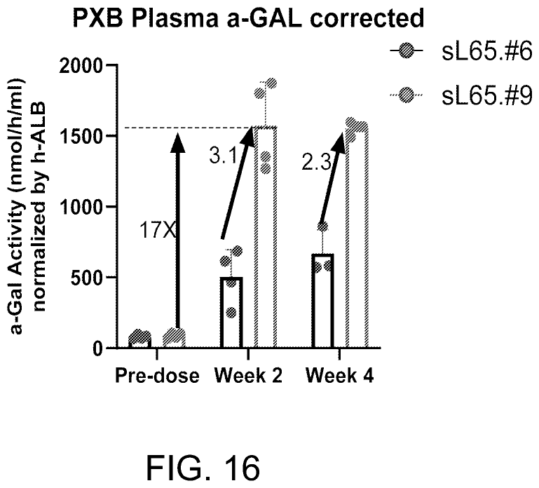

Briefly, constructs 6 and 9 were packaged in an AAV particle comprising a liver tropic capsid protein SL65, and delivered to PXB mice (n=4 per group) via IV administration. Constructs 6 and 9 in Example 6, and the Figures referenced therein, refer to constructs 6B-9B, or SEQ ID Nos: 56 and 59, respectively (see Table 6). Compositions were administered at a dose of 3E13 vg/kg. Plasma samples were harvested pre-dose, and at Week 2 and Week 4. The animals were sacrificed after Week 4. α-GAL activity was measured in plasma and levels of human Albumin (ALB) was evaluated as a control (FIG. 15). As shown in FIG. 15, relative to pre-dose, mice receiving either constructs 6 or 9 had a significant increase in the level of α-GAL activity, while no significant change was observed for ALB levels. FIG. 16 shows the corrected α-GAL activity level normalized by the ALB level. Specifically, a 17-fold increase in the level of α-GAL activity was observed in mice two weeks post administration of construct 9 packaged in AAV particles with SL65 capsid.

The present example demonstrates, among other things, that constructs of the present disclosure produce mature, functional GAL in vivo, and the level and/or activity of GAL were significantly improved in mice receiving the constructs of the present disclosure.

### EQUIVALENTS AND SCOPE

Those skilled in the art will recognize, or be able to ascertain using no more than routine experimentation, many equivalents to the specific embodiments and methods described herein. Such equivalents are intended to be encompassed by the scope of the following claims.

TABLE 6
Sequences of the Disclosure

| | | SEQ |
|---|---|---|
| | | ID |
| | Sequence | NO: |
| GAL | LDNGLARTPTMGWLHWERFMCNLDCQEEPDSCISEKLFMEMAELMVSEGWKDAGYEYLCIDDCWMAPQRDSEGRLOADPQRFPHGIRQ | 1 |
| mature | LANYVHSKGLKLGIYADVGNKTCAGFPGSFGYYDIDAQTFADWGVDLLKFDGCYCDSLENLADGYKHMSLALNRTGRSIVYSCEWPLY | |
| protein | MWPFQKPNYTEIRQYCNHWRNFADIDDSWKSIKSILDWTSFNQERIVDVAGPGGWNDPDMLVIGNFGLSWNQQVTQMALWAIMAAPLF | |
| sequence | MSNDLRHISPQAKALLQDKDVIAINQDPLGKQGYQLRQGDNFEVWERPLSGLAWAVAMINRQEIGGPRSYTIAVASLGKGVACNPACE | |
| (32- | ITQLLPVKRKLGFYEWTSRLRSHINPTGTVLLQLENTMQMSLKDLL | |
| 429 aa | | |
| of NP_ | | |
| 000160) | | |
| GAL mature | CTGGACAATGGATTGGCAAGGACGCCTACCATGGGCTGGCTGCACTGGGAGCGCTTCATGTGCAACCTTGACTGCCAGGAAGAGCCAG | 2 |
| peptide | ATTCCTGCATCAGTGAGAAGCTCTTCATGGAGATGGCAGAGCTCATGGTCTCAGAAGGCTGGAAGGATGCAGGTTATGAGTACCTCTG | |
| nucleic | CATTGATGACTGTTGGATGGCTCCCCAAAGAGATTCAGAAGGCAGACTTCAGGCAGACCCTCAGCGCTTTCCTCATGGGATTCGCCAG | |
| acid | CTAGCTAATTATGTTCACAGCAAAGGACTGAAGCTAGGGATTTATGCAGATGTTGGAAATAAAACCTGCGCAGGCTTCCCTGGGAGTT | |
| sequence | TTGGATACTACGACATTGATGCCCAGACCTTTGCTGACTGGGGAGTAGATCTGCTAAAATTTGATGGTTGTTACTGTGACAGTTTGGA | |
| | AAATTTGGCAGATGGTTATAAGCACATGTCCTTGGCCCTGAATAGGACTGGCAGAAGCATTGTGTACTCCTGTGAGTGGCCTCTTTAT | |
| | ATGTGGCCCTTTCAAAAGCCCAATTATACAGAAATCCGACAGTACTGCAATCACTGGCGAAATTTTGCTGACATTGATGATTCCTGGA | |
| | AAAGTATAAAGAGTATCTTGGACTGGACATCTTTTAACCAGGAGAGAATTGTTGATGTTGCTGGACCAGGGGGTTGGAATGACCCAGA | |
| | TATGTTAGTGATTGGCAACTTTGGCCTCAGCTGGAATCAGCAAGTAACTCAGATGGCCCTCTGGGCTATCATGGCTGCTCCTTTATTC | |
| | ATGTCTAATGACCTCCGACACATCAGCCCTCAAGCCAAAGCTCTCCTTCAGGATAAGGACGTAATTGCCATCAATCAGGACCCCTTGG | |
| | GCAAGCAAGGGTACCAGCTTAGACAGGGAGACAACTTTGAAGTGTGGGAACGACCTCTCTCAGGCTTAGCCTGGGCTGTAGCTATGAT | |
| | AAACCGGCAGGAGATTGGTGGACCTCGCTCTTATACCATCGCAGTTGCTTCCCTGGGTAAAGGAGTGGCCTGTAATCCTGCCTGCTTC | |
| | ATCACACAGCTCCTCCCTGTGAAAAGGAAGCTAGGGTTCTATGAATGGACTTCAAGGTTAAGAAGTCACATAAATCCCACAGGCACTG | |
| | TTTTGCTTCAGCTAGAAAATACAATGCAGATGTCATTAAAAGACTTACTTTAA | |
| GAL mature | CTGGATAATGGCCTGGCCAGAACACCTACAATGGGCTGGCTGCACTGGGAGAGATTCATGTGCAACCTGGACTGCCAAGAGGAACCCG | 3 |
| peptide | ACAGCTGCATCAGCGAGAAGCTGTTCATGGAAATGGCCGAGCTGATGGTGTCCGAAGGCTGGAAGGATGCCGGCTACGAGTACCTGTG | |
| codon | CATCGACGACTGTTGGATGGCCCCTCAGAGAGACTCTGAGGGCAGACTGCAGGCCGATCCTCAGAGATTTCCCCACGGCATTAGACAG | |
| optimized | CTGGCCAACTACGTGCACAGCAAGGGCCTGAAGCTGGGCATCTATGCCGACGTGGGCAACAAGACCTGTGCCGGCTTTCCTGGCAGCT | |
| nucleic | TCGGCTACTACGATATCGACGCCCAGACCTTCGCCGATTGGGGAGTCGATCTGCTGAAGTTCGACGGCTGCTACTGCGACAGCCTGGA | |
| acid | AAATCTGGCCGACGGCTACAAGCACATGTCTCTGGCCCTGAATCGGACCGGCAGATCCATCGTGTACAGCTGCGAGTGGCCCCTGTAC | |
| sequence | ATGTGGCCCTTCCAGAAGCCTAACTACACCGAGATCAGACAGTACTGCAACCACTGGCGGAACTTCGCCGACATCGACGATAGCTGGA | |
| version A | AGTCCATCAAGAGCATCCTGGACTGGACCAGCTTCAATCAAGAGCGGATCGTGGACGTGGCAGGACCTGGCGGATGGAACGATCCTGA | |
| | CATGCTGGTCATCGGCAACTTCGGCCTGAGCTGGAACCAGCAAGTGACCCAGATGGCCCTGTGGGCCATTATGGCCGCTCCTCTGTTC | |
| | ATGAGCAACGACCTGAGACACATCAGCCCTCAGGCCAAGGCTCTGCTGCAGGACAAGGATGTGATCGCTATCAACCAGGATCCTCTGG | |
| | GCAAGCAGGGCTACCAGCTGAGACAGGGCGACAATTTCGAAGTGTGGGAAAGACCCCTGAGCGGACTGGCTTGGGCCGTCGCCATGAT | |
| | CAACAGACAAGAGATCGGCGGACCCCGGTCCTACACAATTGCCGTGGCTTCTCTCGGCAAAGGCGTGGCCTGTAATCCCGCCTGCTTT | |
| | ATCACACAGCTGCTGCCCGTGAAGAGAAAGCTGGGCTTTTACGAGTGGACCAGCAGACTGCGGAGCCACATCAATCCTACCGGCACAG | |
| | TGCTGCTGCAGCTGGAAAACACAATGCAGATGAGCCTGAAGGACCTGCTGTGA | |
| GAL mature | CTCGATAATGGGCTCGCTCGGACTCCAACCATGGGCTGGCTCCACTGGGAACGCTTCATGTGTAATCTGGACTGCCAAGAAGAACCAG | 4 |
| peptide | ATAGCTGTATCAGCGAAAAACTGTTTATGGAAATGGCTGAGCTCATGGTCAGCGAGGGCTGGAAGGACGCCGGCTACGAGTATCTGTG | |
| codon | TATCGACGACTGCTGGATGGCCCCTCAGCGGGATTCAGAGGGCAGGCTCCAGGCTGACCCACAGCGGTTTCCCCACGGAATTCGGCAG | |
| optimized | CTGGCCAATTACGTGCACAGCAAAGGCCTGAAGCTGGGGATCTACGCTGATGTGGGGAACAAGACCTGCGCTGGATTTCCAGGCAGCT | |
| nucleic | TCGGCTACTACGATATCGACGCACAGACCTTCGCCGATTGGGGCGTCGACCTCCTGAAATTTGATGGATGCTATTGCGATAGCCTCGA | |
| acid | AAACCTGGCCGACGGGTACAAACACATGAGCCTCGCTCTGAACCGGACCGGCCGGAGCATCGTGTACAGCTGCGAATGGCCTCTGTAC | |
| sequence | ATGTGGCCCTTCCAGAAGCCAAATTACACAGAGATTAGACAGTATTGCAATCATTGGCGGAACTTCGCCGACATCGATGACAGCTGGA | |
| version B | AGAGCATCAAGAGCATCCTCGACTGGACCTCATTCAATCAGGAACGCATTGTGGACGTGGCAGGACCAGGCGGGTGGAATGATCCCGA | |
| | CATGCTGGTGATTGGCAACTTTGGACTGAGCTGGAATCAGCAGGTCACCCAGATGGCACTGTGGGCTATCATGGCCGCCCCCCTGTTT | |
| | ATGAGCAACGACCTCAGGCACATCTCACCACAAGCCAAGGCACTGCTCCAGGATAAGGATGTCATTGCAATTAATCAGGATCCACTGG | |
| | GCAAACAAGGCTATCAGCTGAGACAGGGAGATAATTTCGAAGTGTGGGAGCGGCCTCTGAGCGGACTGGCATGGGCCGTGGCTATGAT | |
| | CAACAGACAGGAGATCGGCGGCCCAAGATCATACACAATCGCCGTGGCATCACTGGGCAAGGGAGTCGCCTGCAACCCCGCATGCTTT | |
| | ATCACTCAGCTGCTGCCTGTGAAAAGGAAGCTGGGCTTCTACGAATGGACAAGCAGACTGAGGAGCCACATCAACCCCACAGGAACAG | |
| | TCCTGCTCCAACTGGAAAATACTATGCAAATGTCACTGAAAGATCTGCTGTGA | |
| GAL mature | CTGGACAATGGGCTGGCCAGGACTCCAACCATGGGCTGGCTCCACTGGGAGAGGTTCATGTGTAATCTGGACTGCCAAGAAGAACCAG | 5 |
| peptide | ATAGCTGTATCTCTGAGAAACTGTTTATGGAAATGGCTGAGCTCATGGTCAGCGAGGGCTGGAAGGATGCCGGCTACGAGTATCTGTG | |
| codon | TATCGATGACTGCTGGATGGCCCCTCAGAGGGATTCAGAGGGCAGGCTCCAGGCTGACCCACAGAGGTTTCCCCATGGCATCAGGCAG | |
| optimized | CTGGCCAATTACGTGCACAGCAAAGGCCTGAAGCTGGGGATCTATGCCGATGTGGGGAACAAGACCTGTGCCGGATTTCCAGGCAGCT | |
| nucleic | TTGGCTACTATGACATCGATGCCCAGACCTTTGCCGATTGGGGGGTGGATCTCCTGAAATTTGATGGATGCTATTGTGACAGCCTGGA | |
| acid | GAACCTGGCCGATGGCTACAAACACATGAGCCTGGCCCTGAACAGGACCGGCAGGAGCATCGTGTACAGCTGTGAGTGGCCTCTGTAC | |
| sequence | ATGTGGCCCTTCCAGAAGCCAAATTACACAGAGATTAGACAGTATTGCAATCATTGGAGGAACTTTGCCGACATTGACGACAGCTGGA | |
| version B | AGAGCATCAAGAGCATCCTGGACTGGACCTCATTCAATCAGGAGAGGATTGTGGATGTGGCAGGACCAGGGGGCTGGAATGATCCTGA | |
| CpG | CATGCTGGTGATTGGCAACTTTGGACTGAGCTGGAATCAGCAGGTCACCCAGATGGCACTGTGGGCTATCATGGCCGCCCCCCTGTTT | |
| reduced | ATGAGCAATGACCTCAGGCACATCTCACCACAAGCCAAGGCACTGCTCCAGGATAAGGATGTCATTGCAATTAATCAGGATCCACTGG | |
| | GCAAACAAGGCTATCAGCTGAGACAGGGAGATAATTTTGAGGTGTGGGAGAGGCCTCTGTCTGGCCTGGCATGGGCCGTGGCTATGAT | |
| | CAACAGACAGGAGATCGGCGGCCCAAGATCATACACAATCGCCGTGGCATCACTGGGCAAGGGAGTGGCCTGCAACCCTGCCTGCTTT | |
| | ATCACTCAGCTGCTGCCTGTGAAAAGGAAGCTGGGCTTCTATGAGTGGACAAGCAGACTGAGGAGCCACATCAACCCCACAGGAACAG | |
| | TCCTGCTCCAACTGGAAAATACTATGCAAATGTCACTGAAAGATCTGCTGTGA | |
| GAL native | MQLRNPELHL GCALALRFLA LVSWDIPGAR A | 6 |
| signal | | |
| peptide | | |
| GAL native | ATGCAGCTGAGGAACCCAGAACTACATCTGGGCTGCGCGCTTGCGCTTCGCTTCCTGGCCCTCGTTTCCTGGGACATCCCTGGGGCTA | 7 |
| signal | GAGCA | |
| peptide | | |
| coding | | |
| sequence | | |
| GAL native | ATGCAGCTGAGAAATCCTGAGCTGCACCTGGGCTGTGCCCTGGCTCTGAGATTTCTGGCTCTGGTGTCCTGGGACATCCCTGGCGCTA | 8 |
| signal | GAGCC | |
| peptide | | |
| codon | | |
| optimized | | |
| version A | | |
| GAL native | ATGCAGCTCAGAAACCCAGAACTCCATCTGGGGTGCGCTCTCGCCCTCCGCTTCCTGGCACTCGTCAGCTGGGATATTCCTGGGGCTA | 9 |
| signal | GAGCC | |
| peptide | | |
| codon | | |
| optimized | | |
| version B | | |
| GAL native | ATGCAGCTCAGAAACCCAGAACTCCATCTGGGGTGTGCCCTGGCCCTGAGGTTCCTGGCACTGGTGAGCTGGGATATTCCTGGGGCTA | 10 |
| signal | GAGCC | |
| peptide | | |
| codon | | |
| optimized | | |
| version B | | |
| CpG | | |
| reduced | | |
| Human and | MGWSCIILFLVATATGVHS | 11 |
| mouse IgG1 | | |
| signal | | |
| peptide | | |
| MIgG1 | | |
| (Genbank | | |
| ID: | | |
| AAA51634) | | |
| and hIgG1 | | |
| (GenBank | | |
| ID: | | |
| QBK47409 | | |
| heavy | | |
| chain | | |
| Human IgG1 | ATGGGCTGGAGCTGCATCATCCTGTTCCTGGTGGCCACCGCCACCGGCGTGCACAGC | 12 |
| reverse | | |
| translated | | |
| coding | | |
| sequence | | |
| Human IgG1 | ATGGGGTGGTCATGTATTATCCTGTTTCTGGTCGCTACCGCAACCGGAGTGCACAGC | 13 |
| codon | | |
| optimized | | |
| C | | |
| Human IgG1 | ATGGGGTGGTCATGTATTATCCTGTTTCTGGTGGCCACAGCCACAGGCGTGCACAGC | 14 |
| codon | | |
| optimized | | |
| C CpG | | |
| reduced | | |
| Synthetic | MGWSCILFLVATATGVHS | 15 |
| IgG1 | | |
| signal | | |
| peptide | | |
| Synthetic | ATGGGCTGGAGCTGCATTCTGTTTCTGGTGGCGACCGCGACCGGCGTGCATAGC | 16 |
| IgG1 | | |
| reverse | | |
| transcribed | | |
| 5′ ITR | TTGGCCACTCCCTCTCTGCGCGCTCGCTCGCTCACTGAGGCCGCCCGGGCAAAGCCCGGGCGTCGGGCGACCTTTGGTCGCCCGGCCT | 17 |
| | CAGTGAGCGAGCGAGCGCGCAGAGAGGGAGTGGCCAACTCCATCACTAGGGGTTCCT | |
| 3′ ITR | AGGAACCCCTAGTGATGGAGTTGGCCACTCCCTCTCTGCGCGCTCGCTCGCTCACTGAGGCCGGGCGACCAAAGGTCGCCCGACGCCC | 18 |
| | GGGCTTTGCCCGGGCGGCCTCAGTGAGCGAGCGAGCGCGCAGAGAGGGAGTGGCCAA | |
| Apo E/C-I | AGGCTCAGAGGCACACAGGAGTTTCTGGGCTCACCCTGCCCCCTTCCAACCCCTCAGTTCCCATCCTCCAGCAGCTGTTTGTGTGCTG | 19 |
| | CCTCTGAAGTCCACACTGAACAAACTTCAGCCTACTCATGTCCCTAAAATGGGCAAACATTGCAAGCAGCAAACAGCAAACACACAGC | |
| | CCTCCCTGCCTGCTGACCTTGGAGCTGGGGCAGAGGTCAGAGACCTCTCTGGGCCCATGCCACCTCCAACATCCACTCGACCCCTTGG | |
| | AATTTCGGTGGAGAGGAGCAGAGGTTGTCCTGGCGTGGTTTAGGTAGTGTGAGAGGG | |
| A1AT | GATCTTGCTACCAGTGGAACAGCCACTAAGGATTCTGCAGTGAGAGCAGAGGGCCAGCTAAGTGGTACTCTCCCAGAGACTGTCTGAC | 20 |
| promoter | TCACGCCACCCCCTCCACCTTGGACACAGGACGCTGTGGTTTCTGAGCCAGGTACAATGACTCCTTTCGGTAAGTGCAGTGGAAGCTG | |
| | TACACTGCCCAGGCAAAGCGTCCGGGCAGCGTAGGCGGGCGACTCAGATCCCAGCCAGTGGACTTAGCCCCTGTTTGCTCCTCCGATA | |
| | ACTGGGGTGACCTTGGTTAATATTCACCAGCAGCCTCCCCCGTTGCCCCTCTGGATCCACTGCTTAAATACGGACGAGGACAGGGCCC | |
| | TGTCTCCTCAGCTTCAGGCACCACCACTGACCTGGGACAGT | |
| b-globin | GTGAGTCTATGGGACCCTTGATGTTTTCTTTCCCCTTCTTTTCTATGGTTAAGTTCATGTCATAGGAAGGGGAGAAGTAACAGGGTAC | 21 |
| intron | ACATATTGACCAAATCAGGGTAATTTTGCATTTGTAATTTTAAAAAATGCTTTCTTCTTTTAATATACTTTTTTGTTTATCTTATTTC | |
| | TAATACTTTCCCTAATCTCTTTCTTTCAGGGCAATAATGATACAATGTATCATGCCTCTTTGCACCATTCTAAAGAATAACAGTGATA | |
| | ATTTCTGGGTTAAGGCAATAGCAATATTTCTGCATATAAATATTTCTGCATATAAATTGTAACTGATGTAAGAGGTTTCATATTGCTA | |
| | ATAGCAGCTACAATCCAGCTACCATTCTGCTTTTATTTTCTGGTTGGGATAAGGCTGGATTATTCTGAGTCCAAGCTAGGCCCTTTTG | |
| | CTAATCTTGTTCATACCTCTTATCTTCCTCCCACAG | |
| Kozak | GCCGCCACCATG | 22 |
| sequence | | |
| Bovine | CTGTGCCTTCTAGTTGCCAGCCATCTGTTGTTTGCCCCTCCCCCGTGCCTTCCTTGACCCTGGAAGGTGCCACTCCCACTGTCCTTTC | 23 |
| growth | CTAATAAAATGAGGAAATTGCATCGCATTGTCTGAGTAGGTGTCATTCTATTCTGGGGGGTGGGGTGGGGCAGGACAGCAAGGGGGAG | |
| hormone | GATTGGGAAGACAATAGCAGGCATGCTGGGGATGCGGTGGGCTCTATGG | |
| poly A | | |
| SV40 polyA | GCTTTATTTGTGAAATTTGTGATGCTATTGCTTTATTTGTAACCATTATAAGCTGCAATAAACAAGTTAACAACAACAATTGCATTCA | 24 |
| | TTTTATGTTTCAGGTTCAGGGGGAGATGTGGGAGGTTTTTTAAAGC | |
| WPRE 590bp | TAATCAACCTCTGGATTACAAAATTTGTGAAAGATTGACTGGTATTCTTAACTATGTTGCTCCTTTTACGCTATGTGGATACGCTGCT | 25 |
| | TTAATGCCTTTGTATCATGCTATTGCTTCCCGTATGGCTTTCATTTTCTCCTCCTTGTATAAATCCTGGTTGCTGTCTCTTTATGAGG | |
| | AGTTGTGGCCCGTTGTCAGGCAACGTGGCGTGGTGTGCACTGTGTTTGCTGACGCAACCCCCACTGGTTGGGGCATTGCCACCACCTG | |
| | TCAGCTCCTTTCCGGGACTTTCGCTTTCCCCCTCCCTATTGCCACGGCGGAACTCATCGCCGCCTGCCTTGCCCGCTGCTGGACAGGG | |
| | GCTCGGCTGTTGGGCACTGACAATTCCGTGGTGTTGTCGGGGAAATCATCGTCCTTTCCTTGGCTGCTCGCCTGTGTTGCCACCTGGA | |
| | TTCTGCGCGGGACGTCCTTCTGCTACGTCCCTTCGGCCCTCAATCCAGCGGACCTTCCTTCCCGCGGCCTGCTGCCGGCTCTGCGGCC | |
| | TCTTCCGCGTCTTCGCCTTCGCCCTCAGACGAGTCGGATCTCCCTTTGGGCCGCCTCCCCGC | |
| W3SL 249bp | TAATCAACCTCTGGATTACAAAATTTGTGAAAGATTGACTGGTATTCTTAACTATGTTGCTCCTTTTACGCTATGTGGATACGCTGCT | 26 |
| | TTAATGCCTTTGTATCATGCTATTGCTTCCCGTATGGCTTTCATTTTCTCCTCCTTGTATAAATCCTGGTTAGTTCTTGCCACGGCGG | |
| | AACTCATCGCCGCCTGCCTTGCCCGCTGCTGGACAGGGGCTCGGCTGTTGGGCACTGACAATTCCGTGGTGTT | |
| GAL with | MQLRNPELHLGCALALRFLALVSWDIPGARALDNGLARTPTMGWLHWERFMCNLDCQEEPDSCISEKLEMEMAELMVSEGWKDAGYEY | 27 |
| native | LCIDDCWMAPQRDSEGRLOADPQRFPHGIRQLANYVHSKGLKLGIYADVGNKTCAGFPGSFGYYDIDAQTFADWGVDLLKEDGCYCDS | |
| signal | LENLADGYKHMSLALNRTGRSIVYSCEWPLYMWPFQKPNYTEIRQYCNHWRNFADIDDSWKSIKSILDWTSFNQERIVDVAGPGGWND | |
| | PDMLVIGNFGLSWNQQVTQMALWAIMAAPLFMSNDLRHISPQAKALLQDKDVIAINQDPLGKOGYQLROGDNFEVWERPLSGLAWAVA | |
| | MINRQEIGGPRSYTIAVASLGKGVACNPACFITQLLPVKRKLGFYEWTSRLRSHINPTGTVLLQLENTMQMSLKDLL* | |
| GAL with | MGWSCIILFLVATATGVHSLDNGLARTPTMGWLHWERFMCNLDCQEEPDSCISEKLFMEMAELMVSEGWKDAGYEYLCIDDCWMAPQR | 28 |
| IgG1 | DSEGRLQADPQRFPHGIRQLANYVHSKGLKLGIYADVGNKTCAGFPGSFGYYDIDAQTFADWGVDLLKFDGCYCDSLENLADGYKHMS | |
| signal | LALNRTGRSIVYSCEWPLYMWPFQKPNYTEIRQYCNHWRNFADIDDSWKSIKSILDWTSFNQERIVDVAGPGGWNDPDMLVIGNEGLS | |
| | WNQQVTQMALWAIMAAPLFMSNDLRHISPQAKALLQDKDVIAINQDPLGKQGYQLRQGDNFEVWERPLSGLAWAVAMINRQEIGGPRS | |
| | YTIAVASLGKGVACNPACFITQLLPVKRKLGFYEWTSRLRSHINPTGTVLLQLENTMQMSLKDLL* | |
| GAL | >NP_000160.1 alpha-galactosidase A precursor [Homo sapiens] | 29 |
| protein | MQLRNPELHLGCALALRFLALVSWDIPGARALDNGLARTPTMGWLHWERFMCNLDCQEEPDSCISEKLFM | |
| NP_000160. | EMAELMVSEGWKDAGYEYLCIDDCWMAPQRDSEGRLOADPQRFPHGIRQLANYVHSKGLKLGIYADVGNK | |
| 1 | TCAGFPGSFGYYDIDAQTFADWGVDLLKFDGCYCDSLENLADGYKHMSLALNRTGRSIVYSCEWPLYMWP | |
| | FQKPNYTEIRQYCNHWRNFADIDDSWKSIKSILDWTSFNQERIVDVAGPGGWNDPDMLVIGNFGLSWNQQ | |
| | VTQMALWAIMAAPLFMSNDLRHISPQAKALLQDKDVIAINQDPLGKQGYQLRQGDNFEVWERPLSGLAWA | |
| | VAMINRQEIGGPRSYTIAVASLGKGVACNPACFITQLLPVKRKLGFYEWTSRLRSHINPTGTVLLQLENT | |
| | MQMSLKDLL | |
| GLA | >NM_000169.3 Homo sapiens galactosidase alpha (GLA), transcript variant 1, mRNA | 30 |
| NM_000169. | ATGCTGTCCGGTCACCGTGACAATGCAGCTGAGGAACCCAGAACTACATCTGGGCTGCGCGCTTGCGCTT | |
| 3 | CGCTTCCTGGCCCTCGTTTCCTGGGACATCCCTGGGGCTAGAGCACTGGACAATGGATTGGCAAGGACGC | |
| | CTACCATGGGCTGGCTGCACTGGGAGCGCTTCATGTGCAACCTTGACTGCCAGGAAGAGCCAGATTCCTG | |
| | CATCAGTGAGAAGCTCTTCATGGAGATGGCAGAGCTCATGGTCTCAGAAGGCTGGAAGGATGCAGGTTAT | |
| | GAGTACCTCTGCATTGATGACTGTTGGATGGCTCCCCAAAGAGATTCAGAAGGCAGACTTCAGGCAGACC | |
| | CTCAGCGCTTTCCTCATGGGATTCGCCAGCTAGCTAATTATGTTCACAGCAAAGGACTGAAGCTAGGGAT | |
| | TTATGCAGATGTTGGAAATAAAACCTGCGCAGGCTTCCCTGGGAGTTTTGGATACTACGACATTGATGCC | |
| | CAGACCTTTGCTGACTGGGGAGTAGATCTGCTAAAATTTGATGGTTGTTACTGTGACAGTTTGGAAAATT | |
| | TGGCAGATGGTTATAAGCACATGTCCTTGGCCCTGAATAGGACTGGCAGAAGCATTGTGTACTCCTGTGA | |
| | GTGGCCTCTTTATATGTGGCCCTTTCAAAAGCCCAATTATACAGAAATCCGACAGTACTGCAATCACTGG | |
| | CGAAATTTTGCTGACATTGATGATTCCTGGAAAAGTATAAAGAGTATCTTGGACTGGACATCTTTTAACC | |
| | AGGAGAGAATTGTTGATGTTGCTGGACCAGGGGGTTGGAATGACCCAGATATGTTAGTGATTGGCAACTT | |
| | TGGCCTCAGCTGGAATCAGCAAGTAACTCAGATGGCCCTCTGGGCTATCATGGCTGCTCCTTTATTCATG | |
| | TCTAATGACCTCCGACACATCAGCCCTCAAGCCAAAGCTCTCCTTCAGGATAAGGACGTAATTGCCATCA | |
| | ATCAGGACCCCTTGGGCAAGCAAGGGTACCAGCTTAGACAGGGAGACAACTTTGAAGTGTGGGAACGACC | |
| | TCTCTCAGGCTTAGCCTGGGCTGTAGCTATGATAAACCGGCAGGAGATTGGTGGACCTCGCTCTTATACC | |
| | ATCGCAGTTGCTTCCCTGGGTAAAGGAGTGGCCTGTAATCCTGCCTGCTTCATCACACAGCTCCTCCCTG | |
| | TGAAAAGGAAGCTAGGGTTCTATGAATGGACTTCAAGGTTAAGAAGTCACATAAATCCCACAGGCACTGT | |
| | TTTGCTTCAGCTAGAAAATACAATGCAGATGTCATTAAAAGACTTACTTTAAAATGTT | |
| Construct | 5′ITR (SEQ ID NO: 17), Apo E/C-I (SEQ ID NO: 19), A1AT (SEQ ID NO: 20), | 31 |
| 1A | β-globin intron (SEQ ID NO: 21), KOZAK consensus leader sequence (SEQ ID NO: 22), | |
| | hGLA signal peptide (SEQ ID 7), mature peptide (SEQ ID 2), bGH polyA | |
| | 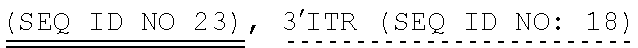 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGCAGCTGAGGAACCCAGAACTACATCTGGGCTGCGCGCTTGCGCTTCGCTTCCTGGCCCTCGT | |
| | TTCCTGGGACATCCCTGGGGCTAGAGCACTGGACAATGGATTGGCAAGGACGCCTACCATGGGCTGGCTGCACTGGGAGCGCTT | |
| | CATGTGCAACCTTGACTGCCAGGAAGAGCCAGATTCCTGCATCAGTGAGAAGCTCTTCATGGAGATGGCAGAGCTCATGGTCTC | |
| | AGAAGGCTGGAAGGATGCAGGTTATGAGTACCTCTGCATTGATGACTGTTGGATGGCTCCCCAAAGAGATTCAGAAGGCAGAC | |
| | TTCAGGCAGACCCTCAGCGCTTTCCTCATGGGATTCGCCAGCTAGCTAATTATGTTCACAGCAAAGGACTGAAGCTAGGGATTTA | |
| | TGCAGATGTTGGAAATAAAACCTGCGCAGGCTTCCCTGGGAGTTTTGGATACTACGACATTGATGCCCAGACCTTTGCTGACTGG | |
| | GGAGTAGATCTGCTAAAATTTGATGGTTGTTACTGTGACAGTTTGGAAAATTTGGCAGATGGTTATAAGCACATGTCCTTGGCCC | |
| | TGAATAGGACTGGCAGAAGCATTGTGTACTCCTGTGAGTGGCCTCTTTATATGTGGCCCTTTCAAAAGCCCAATTATACAGAAAT | |
| | CCGACAGTACTGCAATCACTGGCGAAATTTTGCTGACATTGATGATTCCTGGAAAAGTATAAAGAGTATCTTGGACTGGACATCT | |
| | TTTAACCAGGAGAGAATTGTTGATGTTGCTGGACCAGGGGGTTGGAATGACCCAGATATGTTAGTGATTGGCAACTTTGGCCTC | |
| | AGCTGGAATCAGCAAGTAACTCAGATGGCCCTCTGGGCTATCATGGCTGCTCCTTTATTCATGTCTAATGACCTCCGACACATCAG | |
| | CCCTCAAGCCAAAGCTCTCCTTCAGGATAAGGACGTAATTGCCATCAATCAGGACCCCTTGGGCAAGCAAGGGTACCAGCTTAG | |
| | ACAGGGAGACAACTTTGAAGTGTGGGAACGACCTCTCTCAGGCTTAGCCTGGGCTGTAGCTATGATAAACCGGCAGGAGATTG | |
| | GTGGACCTCGCTCTTATACCATCGCAGTTGCTTCCCTGGGTAAAGGAGTGGCCTGTAATCCTGCCTGCTTCATCACACAGCTCCTC | |
| | CCTGTGAAAAGGAAGCTAGGGTTCTATGAATGGACTTCAAGGTTAAGAAGTCACATAAATCCCACAGGCACTGTTTTGCTTCAG | |
| | CTAGAAAATACAATGCAGATGTCATTAAAAGACTTACTTTAAgctagctcgagcgaCTGTGCCTTCTAGTTGCCAGCCATCTGTTGTTT | |
| | GCCCCTCCCCCGTGCCTTCCTTGACCCTGGAAGGTGCCACTCCCACTGTCCTTTCCTAATAAAATGAGGAAATTGCATCGCATTGTC | |
| | TGAGTAGGTGTCATTCTATTCTGGGGGGTGGGGTGGGGCAGGACAGCAAGGGGGAGGATTGGGAAGACAATAGCAGGCATGCT | |
| | 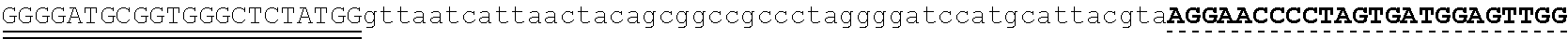 | |
| | 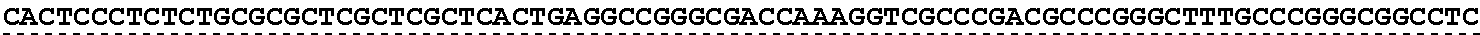 | |
| | 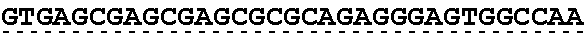 | |
| Construct | 5′ITR (SEQ ID NO: 17), Apo E/C-I (SEQ ID NO: 19), A1AT (SEQ ID NO: 20), | 32 |
| 2A | β-globin intron (SEQ ID NO: 21), KOZAK consensus leader sequence (SEQ ID NO: 22), | |
| | GLA coA signal peptide (SEQ ID 8), coA mature peptide (SEQ ID 3), bGH | |
| | 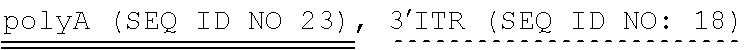 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGCAGCTGAGAAATCCTGAGCTGCACCTGGGCTGTGCCCTGGCTCTGAGATTTCTGGCTCTGGT | |
| | GTCCTGGGACATCCCTGGCGCTAGAGCCCTGGATAATGGCCTGGCCAGAACACCTACAATGGGCTGGCTGCACTGGGAGAGATT | |
| | CATGTGCAACCTGGACTGCCAAGAGGAACCCGACAGCTGCATCAGCGAGAAGCTGTTCATGGAAATGGCCGAGCTGATGGTGT | |
| | CCGAAGGCTGGAAGGATGCCGGCTACGAGTACCTGTGCATCGACGACTGTTGGATGGCCCCTCAGAGAGACTCTGAGGGCAGA | |
| | CTGCAGGCCGATCCTCAGAGATTTCCCCACGGCATTAGACAGCTGGCCAACTACGTGCACAGCAAGGGCCTGAAGCTGGGCATC | |
| | TATGCCGACGTGGGCAACAAGACCTGTGCCGGCTTTCCTGGCAGCTTCGGCTACTACGATATCGACGCCCAGACCTTCGCCGATT | |
| | GGGGAGTCGATCTGCTGAAGTTCGACGGCTGCTACTGCGACAGCCTGGAAAATCTGGCCGACGGCTACAAGCACATGTCTCTGG | |
| | CCCTGAATCGGACCGGCAGATCCATCGTGTACAGCTGCGAGTGGCCCCTGTACATGTGGCCCTTCCAGAAGCCTAACTACACCGA | |
| | GATCAGACAGTACTGCAACCACTGGCGGAACTTCGCCGACATCGACGATAGCTGGAAGTCCATCAAGAGCATCCTGGACTGGAC | |
| | CAGCTTCAATCAAGAGCGGATCGTGGACGTGGCAGGACCTGGCGGATGGAACGATCCTGACATGCTGGTCATCGGCAACTTCG | |
| | GCCTGAGCTGGAACCAGCAAGTGACCCAGATGGCCCTGTGGGCCATTATGGCCGCTCCTCTGTTCATGAGCAACGACCTGAGAC | |
| | ACATCAGCCCTCAGGCCAAGGCTCTGCTGCAGGACAAGGATGTGATCGCTATCAACCAGGATCCTCTGGGCAAGCAGGGCTACC | |
| | AGCTGAGACAGGGCGACAATTTCGAAGTGTGGGAAAGACCCCTGAGCGGACTGGCTTGGGCCGTCGCCATGATCAACAGACAA | |
| | GAGATCGGCGGACCCCGGTCCTACACAATTGCCGTGGCTTCTCTCGGCAAAGGCGTGGCCTGTAATCCCGCCTGCTTTATCACAC | |
| | AGCTGCTGCCCGTGAAGAGAAAGCTGGGCTTTTACGAGTGGACCAGCAGACTGCGGAGCCACATCAATCCTACCGGCACAGTGC | |
| | TGCTGCAGCTGGAAAACACAATGCAGATGAGCCTGAAGGACCTGCTGTGAgctagctcgagcgaCTGTGCCTTCTAGTTGCCAGCCAT | |
| | CTGTTGTTTGCCCCTCCCCCGTGCCTTCCTTGACCCTGGAAGGTGCCACTCCCACTGTCCTTTCCTAATAAAATGAGGAAATTGCATC | |
| | GCATTGTCTGAGTAGGTGTCATTCTATTCTGGGGGGTGGGGTGGGGCAGGACAGCAAGGGGGAGGATTGGGAAGACAATAGCA | |
| | 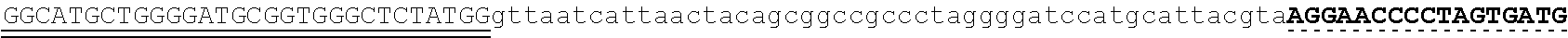 | |
| | 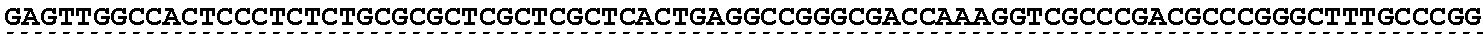 | |
| | 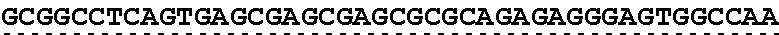 | |
| Construct | 5′ITR (SEQ ID NO: 17), Apo E/C-I (SEQ ID NO: 19), A1AT (SEQ ID NO: 20), | 33 |
| 3A | β-globin intron (SEQ ID NO: 21), KOZAK consensus leader sequence (SEQ ID NO: 22), | |
| | GLA coB signal peptide (SEQ ID 9), coB mature peptide (SEQ ID 4), bGH | |
| | 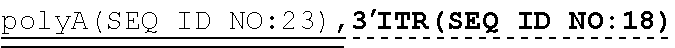 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGCAGCTCAGAAACCCAGAACTCCATCTGGGGTGCGCTCTCGCCCTCCGCTTCCTGGCACTCGTC | |
| | AGCTGGGATATTCCTGGGGCTAGAGCCCTCGATAATGGGCTCGCTCGGACTCCAACCATGGGCTGGCTCCACTGGGAACGCTTCA | |
| | TGTGTAATCTGGACTGCCAAGAAGAACCAGATAGCTGTATCAGCGAAAAACTGTTTATGGAAATGGCTGAGCTCATGGTCAGCG | |
| | AGGGCTGGAAGGACGCCGGCTACGAGTATCTGTGTATCGACGACTGCTGGATGGCCCCTCAGCGGGATTCAGAGGGCAGGCTC | |
| | CAGGCTGACCCACAGCGGTTTCCCCACGGAATTCGGCAGCTGGCCAATTACGTGCACAGCAAAGGCCTGAAGCTGGGGATCTAC | |
| | GCTGATGTGGGGAACAAGACCTGCGCTGGATTTCCAGGCAGCTTCGGCTACTACGATATCGACGCACAGACCTTCGCCGATTGG | |
| | GGCGTCGACCTCCTGAAATTTGATGGATGCTATTGCGATAGCCTCGAAAACCTGGCCGACGGGTACAAACACATGAGCCTCGCTC | |
| | TGAACCGGACCGGCCGGAGCATCGTGTACAGCTGCGAATGGCCTCTGTACATGTGGCCCTTCCAGAAGCCAAATTACACAGAGA | |
| | TTAGACAGTATTGCAATCATTGGCGGAACTTCGCCGACATCGATGACAGCTGGAAGAGCATCAAGAGCATCCTCGACTGGACCT | |
| | CATTCAATCAGGAACGCATTGTGGACGTGGCAGGACCAGGCGGGTGGAATGATCCCGACATGCTGGTGATTGGCAACTTTGGA | |
| | CTGAGCTGGAATCAGCAGGTCACCCAGATGGCACTGTGGGCTATCATGGCCGCCCCCCTGTTTATGAGCAACGACCTCAGGCACA | |
| | TCTCACCACAAGCCAAGGCACTGCTCCAGGATAAGGATGTCATTGCAATTAATCAGGATCCACTGGGCAAACAAGGCTATCAGC | |
| | TGAGACAGGGAGATAATTTCGAAGTGTGGGAGCGGCCTCTGAGCGGACTGGCATGGGCCGTGGCTATGATCAACAGACAGGA | |
| | GATCGGCGGCCCAAGATCATACACAATCGCCGTGGCATCACTGGGCAAGGGAGTCGCCTGCAACCCCGCATGCTTTATCACTCAG | |
| | CTGCTGCCTGTGAAAAGGAAGCTGGGCTTCTACGAATGGACAAGCAGACTGAGGAGCCACATCAACCCCACAGGAACAGTCCT | |
| | GCTCCAACTGGAAAATACTATGCAAATGTCACTGAAAGATCTGCTGTGAgctagctcgagcgaCTGTGCCTTCTAGTTGCCAGCCATCT | |
| | GTTGTTTGCCCCTCCCCCGTGCCTTCCTTGACCCTGGAAGGTGCCACTCCCACTGTCCTTTCCTAATAAAATGAGGAAATTGCATCG | |
| | CATTGTCTGAGTAGGTGTCATTCTATTCTGGGGGGTGGGGGGGGCAGGACAGCAAGGGGGAGGATTGGGAAGACAATAGCAG | |
| | 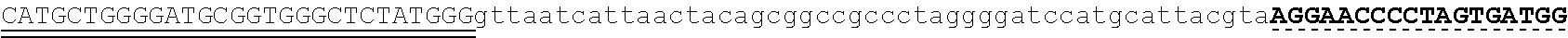 | |
| | 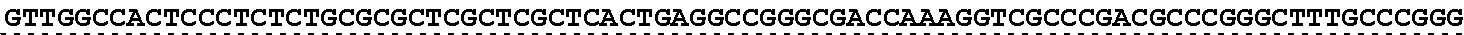 | |
| | 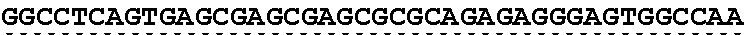 | |
| Construct | 5′ITR (SEQ ID NO: 17), Apo E/C-I (SEQ ID NO: 19), A1AT (SEQ ID NO: 20), | 34 |
| 4A | β-globin intron (SEQ ID NO: 21), KOZAK consensus leader sequence (SEQ ID NO: 22), | |
| | GLA coB-CpG signal peptide (SEQ ID 10), coB-CpG mature peptide (SEQ ID | |
| | 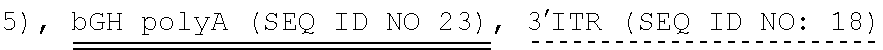 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGCAGCTCAGAAACCCAGAACTCCATCTGGGGTGTGCCCTGGCCCTGAGGTTCCTGGCACTGGT | |
| | GAGCTGGGATATTCCTGGGGCTAGAGCCCTGGACAATGGGCTGGCCAGGACTCCAACCATGGGCTGGCTCCACTGGGAGAGGTT | |
| | CATGTGTAATCTGGACTGCCAAGAAGAACCAGATAGCTGTATCTCTGAGAAACTGTTTATGGAAATGGCTGAGCTCATGGTCAG | |
| | CGAGGGCTGGAAGGATGCCGGCTACGAGTATCTGTGTATCGATGACTGCTGGATGGCCCCTCAGAGGGATTCAGAGGGCAGGC | |
| | TCCAGGCTGACCCACAGAGGTTTCCCCATGGCATCAGGCAGCTGGCCAATTACGTGCACAGCAAAGGCCTGAAGCTGGGGATCT | |
| | ATGCCGATGTGGGGAACAAGACCTGTGCCGGATTTCCAGGCAGCTTTGGCTACTATGACATCGATGCCCAGACCTTTGCCGATTG | |
| | GGGGGTGGATCTCCTGAAATTTGATGGATGCTATTGTGACAGCCTGGAGAACCTGGCCGATGGCTACAAACACATGAGCCTGGC | |
| | CCTGAACAGGACCGGCAGGAGCATCGTGTACAGCTGTGAGTGGCCTCTGTACATGTGGCCCTTCCAGAAGCCAAATTACACAGA | |
| | GATTAGACAGTATTGCAATCATTGGAGGAACTTTGCCGACATTGACGACAGCTGGAAGAGCATCAAGAGCATCCTGGACTGGA | |
| | CCTCATTCAATCAGGAGAGGATTGTGGATGTGGCAGGACCAGGGGGCTGGAATGATCCTGACATGCTGGTGATTGGCAACTTT | |
| | GGACTGAGCTGGAATCAGCAGGTCACCCAGATGGCACTGTGGGCTATCATGGCCGCCCCCCTGTTTATGAGCAATGACCTCAGG | |
| | CACATCTCACCACAAGCCAAGGCACTGCTCCAGGATAAGGATGTCATTGCAATTAATCAGGATCCACTGGGCAAACAAGGCTAT | |
| | CAGCTGAGACAGGGAGATAATTTTGAGGTGTGGGAGAGGCCTCTGTCTGGCCTGGCATGGGCCGTGGCTATGATCAACAGACA | |
| | GGAGATCGGCGGCCCAAGATCATACACAATCGCCGTGGCATCACTGGGCAAGGGAGTGGCCTGCAACCCTGCCTGCTTTATCAC | |
| | TCAGCTGCTGCCTGTGAAAAGGAAGCTGGGCTTCTATGAGTGGACAAGCAGACTGAGGAGCCACATCAACCCCACAGGAACAG | |
| | TCCTGCTCCAACTGGAAAATACTATGCAAATGTCACTGAAAGATCTGCTGTGAgctagctcgagcgaCTGTGCCTTCTAGTTGCCAGCC | |
| | ATCTGTTGTTTGCCCCTCCCCCGTGCCTTCCTTGACCCTGGAAGGTGCCACTCCCACTGTCCTTTCCTAATAAAATGAGGAAATTGCA | |
| | TCGCATTGTCTGAGTAGGTGTCATTCTATTCTGGGGGGTGGGGTGGGGCAGGACAGCAAGGGGGAGGATTGGGAAGACAATAG | |
| | 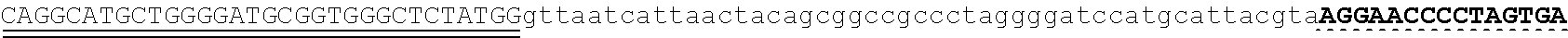 | |
| | 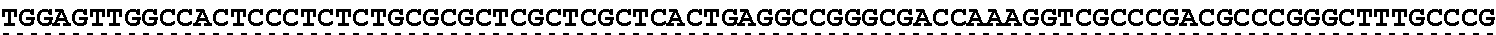 | |
| | 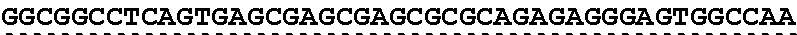 | |
| Construct | 5′ITR (SEQ ID NO: 17), Apo E/C-I (SEQ ID NO: 19), A1AT (SEQ ID NO: 20), | 35 |
| 5A | β-globin intron (SEQ ID NO: 21), KOZAK consensus leader sequence (SEQ ID NO: 22), | |
| | IgG1 signal peptide (SEQ ID 11 and 12), mature peptide (SEQ ID 2), bGH | |
| | 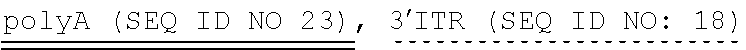 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGGGCTGGAGCTGCATCATCCTGTTCCTGGTGGCCACCGCCACCGGCGTGCACAGCCTGGACA | |
| | ATGGATTGGCAAGGACGCCTACCATGGGCTGGCTGCACTGGGAGCGCTTCATGTGCAACCTTGACTGCCAGGAAGAGCCAGATT | |
| | CCTGCATCAGTGAGAAGCTCTTCATGGAGATGGCAGAGCTCATGGTCTCAGAAGGCTGGAAGGATGCAGGTTATGAGTACCTCT | |
| | GCATTGATGACTGTTGGATGGCTCCCCAAAGAGATTCAGAAGGCAGACTTCAGGCAGACCCTCAGCGCTTTCCTCATGGGATTCG | |
| | CCAGCTAGCTAATTATGTTCACAGCAAAGGACTGAAGCTAGGGATTTATGCAGATGTTGGAAATAAAACCTGCGCAGGCTTCCC | |
| | TGGGAGTTTTGGATACTACGACATTGATGCCCAGACCTTTGCTGACTGGGGAGTAGATCTGCTAAAATTTGATGGTTGTTACTGT | |
| | GACAGTTTGGAAAATTTGGCAGATGGTTATAAGCACATGTCCTTGGCCCTGAATAGGACTGGCAGAAGCATTGTGTACTCCTGT | |
| | GAGTGGCCTCTTTATATGTGGCCCTTTCAAAAGCCCAATTATACAGAAATCCGACAGTACTGCAATCACTGGCGAAATTTTGCTG | |
| | ACATTGATGATTCCTGGAAAAGTATAAAGAGTATCTTGGACTGGACATCTTTTAACCAGGAGAGAATTGTTGATGTTGCTGGAC | |
| | CAGGGGGTTGGAATGACCCAGATATGTTAGTGATTGGCAACTTTGGCCTCAGCTGGAATCAGCAAGTAACTCAGATGGCCCTCT | |
| | GGGCTATCATGGCTGCTCCTTTATTCATGTCTAATGACCTCCGACACATCAGCCCTCAAGCCAAAGCTCTCCTTCAGGATAAGGAC | |
| | GTAATTGCCATCAATCAGGACCCCTTGGGCAAGCAAGGGTACCAGCTTAGACAGGGAGACAACTTTGAAGTGTGGGAACGACC | |
| | TCTCTCAGGCTTAGCCTGGGCTGTAGCTATGATAAACCGGCAGGAGATTGGTGGACCTCGCTCTTATACCATCGCAGTTGCTTCCC | |
| | TGGGTAAAGGAGTGGCCTGTAATCCTGCCTGCTTCATCACACAGCTCCTCCCTGTGAAAAGGAAGCTAGGGTTCTATGAATGGA | |
| | CTTCAAGGTTAAGAAGTCACATAAATCCCACAGGCACTGTTTTGCTTCAGCTAGAAAATACAATGCAGATGTCATTAAAAGACTT | |
| | ACTTTAAgctagctcgagcgaCTGTGCCTTCTAGTTGCCAGCCATCTGTTGTTTGCCCCTCCCCCGTGCCTTCCTTGACCCTGGAAGGTG | |
| | CCACTCCCACTGTCCTTTCCTAATAAAATGAGGAAATTGCATCGCATTGTCTGAGTAGGTGTCATTCTATTCTGGGGGGTGGGGTG | |
| | GGGCAGGACAGCAAGGGGGAGGATTGGGAAGACAATAGCAGGCATGCTGGGGATGCGGTGGGCTCTATGGgttaatcattaactaca | |
| | 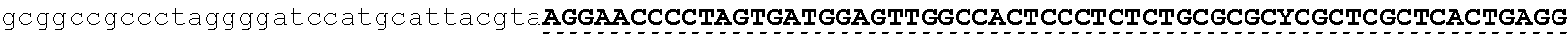 | |
| | 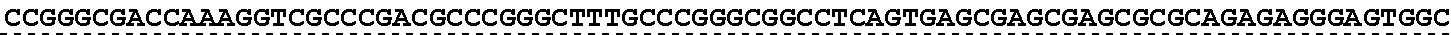 | |
| | 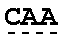 | |
| Construct | 5′ITR (SEQ ID NO: 17), Apo E/C-I (SEQ ID NO: 19), A1AT (SEQ ID NO: 20), | 36 |
| 6A | β-globin intron (SEQ ID NO: 21), KOZAK consensus leader sequence (SEQ ID NO: 22), | |
| | IqG1 coC signal peptide (SEQ ID 13), coA mature peptide (SEQ ID 3), bGH | |
| | 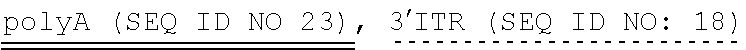 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGGGGTGGTCATGTATTATCCTGTTTCTGGTCGCTACCGCAACCGGAGTGCACAGCCTGGATAA | |
| | TGGCCTGGCCAGAACACCTACAATGGGCTGGCTGCACTGGGAGAGATTCATGTGCAACCTGGACTGCCAAGAGGAACCCGACA | |
| | GCTGCATCAGCGAGAAGCTGTTCATGGAAATGGCCGAGCTGATGGTGTCCGAAGGCTGGAAGGATGCCGGCTACGAGTACCTG | |
| | TGCATCGACGACTGTTGGATGGCCCCTCAGAGAGACTCTGAGGGCAGACTGCAGGCCGATCCTCAGAGATTTCCCCACGGCATT | |
| | AGACAGCTGGCCAACTACGTGCACAGCAAGGGCCTGAAGCTGGGCATCTATGCCGACGTGGGCAACAAGACCTGTGCCGGCTTT | |
| | CCTGGCAGCTTCGGCTACTACGATATCGACGCCCAGACCTTCGCCGATTGGGGAGTCGATCTGCTGAAGTTCGACGGCTGCTACT | |
| | GCGACAGCCTGGAAAATCTGGCCGACGGCTACAAGCACATGTCTCTGGCCCTGAATCGGACCGGCAGATCCATCGTGTACAGCT | |
| | GCGAGTGGCCCCTGTACATGTGGCCCTTCCAGAAGCCTAACTACACCGAGATCAGACAGTACTGCAACCACTGGCGGAACTTCGC | |
| | CGACATCGACGATAGCTGGAAGTCCATCAAGAGCATCCTGGACTGGACCAGCTTCAATCAAGAGCGGATCGTGGACGTGGCAG | |
| | GACCTGGCGGATGGAACGATCCTGACATGCTGGTCATCGGCAACTTCGGCCTGAGCTGGAACCAGCAAGTGACCCAGATGGCCC | |
| | TGTGGGCCATTATGGCCGCTCCTCTGTTCATGAGCAACGACCTGAGACACATCAGCCCTCAGGCCAAGGCTCTGCTGCAGGACAA | |
| | GGATGTGATCGCTATCAACCAGGATCCTCTGGGCAAGCAGGGCTACCAGCTGAGACAGGGCGACAATTTCGAAGTGTGGGAAA | |
| | GACCCCTGAGCGGACTGGCTTGGGCCGTCGCCATGATCAACAGACAAGAGATCGGCGGACCCCGGTCCTACACAATTGCCGTGG | |
| | CTTCTCTCGGCAAAGGCGTGGCCTGTAATCCCGCCTGCTTTATCACACAGCTGCTGCCCGTGAAGAGAAAGCTGGGCTTTTACGA | |
| | GTGGACCAGCAGACTGCGGAGCCACATCAATCCTACCGGCACAGTGCTGCTGCAGCTGGAAAACACAATGCAGATGAGCCTGA | |
| | AGGACCTGCTGTGAgctagctcgagcgaCTGTGCCTTCTAGTTGCCAGCCATCTGTTGTTTGCCCCTCCCCCGTGCCTTCCTTGACCCTG | |
| | GAAGGTGCCACTCCCACTGTCCTTTCCTAATAAAATGAGGAAATTGCATCGCATTGTCTGAGTAGGTGTCATTCTATTCTGGGGGG | |
| | TGGGGTGGGGCAGGACAGCAAGGGGGAGGATTGGGAAGACAATAGCAGGCATGCTGGGGATGCGGTGGGCTCTATGGgttaatc | |
| | 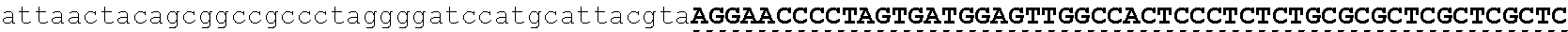 | |
| | 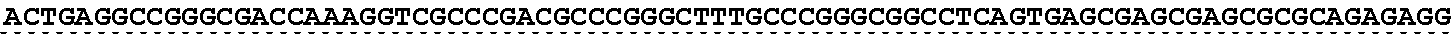 | |
| | 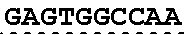 | |
| Construct | 5′ITR (SEQ ID NO: 17), Apo E/C-I (SEQ ID NO: 19), A1AT (SEQ ID NO: 20), | 37 |
| 7A | β-globin intron (SEQ ID NO: 21), KOZAK consensus leader sequence (SEQ ID NO: 22), | |
| | IqG1 coC signal peptide (SEQ ID 13), coB mature peptide (SEQ ID 4), bGH | |
| | 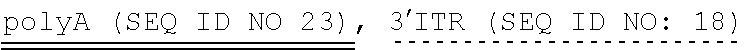 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGGGGTGGTCATGTATTATCCTGTTTCTGGTCGCTACCGCAACCGGAGTGCACAGCCTCGATAA | |
| | TGGGCTCGCTCGGACTCCAACCATGGGCTGGCTCCACTGGGAACGCTTCATGTGTAATCTGGACTGCCAAGAAGAACCAGATAG | |
| | CTGTATCAGCGAAAAACTGTTTATGGAAATGGCTGAGCTCATGGTCAGCGAGGGCTGGAAGGACGCCGGCTACGAGTATCTGT | |
| | GTATCGACGACTGCTGGATGGCCCCTCAGCGGGATTCAGAGGGCAGGCTCCAGGCTGACCCACAGCGGTTTCCCCACGGAATTC | |
| | GGCAGCTGGCCAATTACGTGCACAGCAAAGGCCTGAAGCTGGGGATCTACGCTGATGTGGGGAACAAGACCTGCGCTGGATTT | |
| | CCAGGCAGCTTCGGCTACTACGATATCGACGCACAGACCTTCGCCGATTGGGGCGTCGACCTCCTGAAATTTGATGGATGCTATT | |
| | GCGATAGCCTCGAAAACCTGGCCGACGGGTACAAACACATGAGCCTCGCTCTGAACCGGACCGGCCGGAGCATCGTGTACAGCT | |
| | GCGAATGGCCTCTGTACATGTGGCCCTTCCAGAAGCCAAATTACACAGAGATTAGACAGTATTGCAATCATTGGCGGAACTTCGC | |
| | CGACATCGATGACAGCTGGAAGAGCATCAAGAGCATCCTCGACTGGACCTCATTCAATCAGGAACGCATTGTGGACGTGGCAG | |
| | GACCAGGCGGGTGGAATGATCCCGACATGCTGGTGATTGGCAACTTTGGACTGAGCTGGAATCAGCAGGTCACCCAGATGGCA | |
| | CTGTGGGCTATCATGGCCGCCCCCCTGTTTATGAGCAACGACCTCAGGCACATCTCACCACAAGCCAAGGCACTGCTCCAGGATA | |
| | AGGATGTCATTGCAATTAATCAGGATCCACTGGGCAAACAAGGCTATCAGCTGAGACAGGGAGATAATTTCGAAGTGTGGGAG | |
| | CGGCCTCTGAGCGGACTGGCATGGGCCGTGGCTATGATCAACAGACAGGAGATCGGCGGCCCAAGATCATACACAATCGCCGT | |
| | GGCATCACTGGGCAAGGGAGTCGCCTGCAACCCCGCATGCTTTATCACTCAGCTGCTGCCTGTGAAAAGGAAGCTGGGCTTCTAC | |
| | GAATGGACAAGCAGACTGAGGAGCCACATCAACCCCACAGGAACAGTCCTGCTCCAACTGGAAAATACTATGCAAATGTCACTG | |
| | AAAGATCTGCTGTGAgctagctcgagcgaCTGTGCCTTCTAGTTGCCAGCCATCTGTTGTTTGCCCCTCCCCCGTGCCTTCCTTGACCCT | |
| | GGAAGGTGCCACTCCCACTGTCCTTTCCTAATAAAATGAGGAAATTGCATCGCATTGTCTGAGTAGGTGTCATTCTATTCTGGGGG | |
| | GTGGGGTGGGGCAGGACAGCAAGGGGGAGGATTGGGAAGACAATAGCAGGCATGCTGGGGATGCGGTGGGCTCTATGGgttaat | |
| | 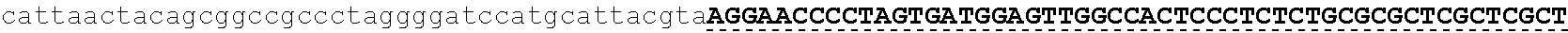 | |
| | 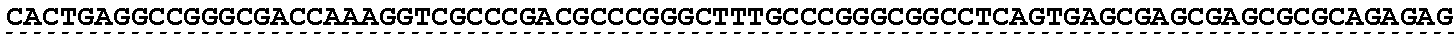 | |
| | 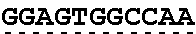 | |
| Construct | 5′ITR (SEQ ID NO: 17), Apo E/C-I (SEQ ID NO: 19), A1AT (SEQ ID NO: 20), | 38 |
| 8A | β-globin intron (SEQ ID NO: 21), KOZAK consensus leader sequence (SEQ ID NO: 22), | |
| | IgG1 coC-CpG signal peptide (SEQ ID 14), coB-CpG mature peptide (SEQ | |
| | 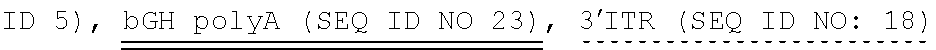 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatca | |
| | AGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAG | |
| | ctcctgggcaacctgctggtctctctgctggcccatcacttt | |
| | ggcaaagcacgcgtcGCCGCCACCATGGGGTGGTCATGTATTATCCTGTTTCTGGTGGCCACAGCCACAGGCGTGCACAGCCTGGACA | |
| | ATGGGCTGGCCAGGACTCCAACCATGGGCTGGCTCCACTGGGAGAGGTTCATGTGTAATCTGGACTGCCAAGAAGAACCAGAT | |
| | AGCTGTATCTCTGAGAAACTGTTTATGGAAATGGCTGAGCTCATGGTCAGCGAGGGCTGGAAGGATGCCGGCTACGAGTATCT | |
| | GTGTATCGATGACTGCTGGATGGCCCCTCAGAGGGATTCAGAGGGCAGGCTCCAGGCTGACCCACAGAGGTTTCCCCATGGCAT | |
| | CAGGCAGCTGGCCAATTACGTGCACAGCAAAGGCCTGAAGCTGGGGATCTATGCCGATGTGGGGAACAAGACCTGTGCCGGAT | |
| | TTCCAGGCAGCTTTGGCTACTATGACATCGATGCCCAGACCTTTGCCGATTGGGGGGTGGATCTCCTGAAATTTGATGGATGCTA | |
| | TTGTGACAGCCTGGAGAACCTGGCCGATGGCTACAAACACATGAGCCTGGCCCTGAACAGGACCGGCAGGAGCATCGTGTACA | |
| | GCTGTGAGTGGCCTCTGTACATGTGGCCCTTCCAGAAGCCAAATTACACAGAGATTAGACAGTATTGCAATCATTGGAGGAACT | |
| | TTGCCGACATTGACGACAGCTGGAAGAGCATCAAGAGCATCCTGGACTGGACCTCATTCAATCAGGAGAGGATTGTGGATGTG | |
| | GCAGGACCAGGGGGCTGGAATGATCCTGACATGCTGGTGATTGGCAACTTTGGACTGAGCTGGAATCAGCAGGTCACCCAGAT | |
| | GGCACTGTGGGCTATCATGGCCGCCCCCCTGTTTATGAGCAATGACCTCAGGCACATCTCACCACAAGCCAAGGCACTGCTCCAG | |
| | GATAAGGATGTCATTGCAATTAATCAGGATCCACTGGGCAAACAAGGCTATCAGCTGAGACAGGGAGATAATTTTGAGGTGTG | |
| | GGAGAGGCCTCTGTCTGGCCTGGCATGGGCCGTGGCTATGATCAACAGACAGGAGATCGGCGGCCCAAGATCATACACAATCG | |
| | CCGTGGCATCACTGGGCAAGGGAGTGGCCTGCAACCCTGCCTGCTTTATCACTCAGCTGCTGCCTGTGAAAAGGAAGCTGGGCT | |
| | TCTATGAGTGGACAAGCAGACTGAGGAGCCACATCAACCCCACAGGAACAGTCCTGCTCCAACTGGAAAATACTATGCAAATG | |
| | TCACTGAAAGATCTGCTGTGAgctagctcgagcgaCTGTGCCTTCTAGTTGCCAGCCATCTGTTGTTTGCCCCTCCCCCGTGCCTTCCTT | |
| | GACCCTGGAAGGTGCCACTCCCACTGTCCTTTCCTAATAAAATGAGGAAATTGCATCGCATTGTCTGAGTAGGTGTCATTCTATTCT | |
| | GGGGGGTGGGGTGGGGCAGGACAGCAAGGGGGAGGATTGGGAAGACAATAGCAGGCATGCTGGGGATGCGGTGGGCTCTAT | |
| | 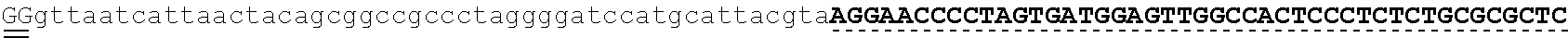 | |
| | 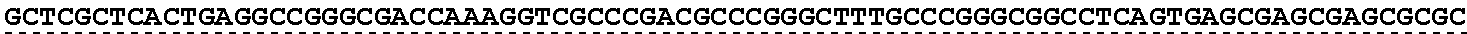 | |
| | 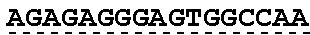 | |
| Construct | 5′ITR (SEQ ID NO: 17), Apo E/C-I (SEQ ID NO: 19), A1AT (SEQ ID NO: 20), | 39 |
| 9A | β-globin intron (SEQ ID NO: 21), KOZAK consensus leader sequence (SEQ ID NO: 22), | |
| | IqG1 coC-CpG signal peptide (SEQ ID 14), coB-CpG mature peptide (SEQ | |
| | 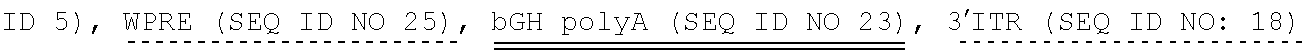 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatcaAGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAGctcctgggcaacctgctggtctctctgctggcccatcacttt | |
| | ggcaaagcacgcgtcGCCGCCACCATGGGGTGGTCATGTATTATCCTGTTTCTGGTGGCCACAGCCACAGGCGTGCACAGCCTGGACA | |
| | ATGGGCTGGCCAGGACTCCAACCATGGGCTGGCTCCACTGGGAGAGGTTCATGTGTAATCTGGACTGCCAAGAAGAACCAGAT | |
| | AGCTGTATCTCTGAGAAACTGTTTATGGAAATGGCTGAGCTCATGGTCAGCGAGGGCTGGAAGGATGCCGGCTACGAGTATCT | |
| | GTGTATCGATGACTGCTGGATGGCCCCTCAGAGGGATTCAGAGGGCAGGCTCCAGGCTGACCCACAGAGGTTTCCCCATGGCAT | |
| | CAGGCAGCTGGCCAATTACGTGCACAGCAAAGGCCTGAAGCTGGGGATCTATGCCGATGTGGGGAACAAGACCTGTGCCGGAT | |
| | TTCCAGGCAGCTTTGGCTACTATGACATCGATGCCCAGACCTTTGCCGATTGGGGGGTGGATCTCCTGAAATTTGATGGATGCTA | |
| | TTGTGACAGCCTGGAGAACCTGGCCGATGGCTACAAACACATGAGCCTGGCCCTGAACAGGACCGGCAGGAGCATCGTGTACA | |
| | GCTGTGAGTGGCCTCTGTACATGTGGCCCTTCCAGAAGCCAAATTACACAGAGATTAGACAGTATTGCAATCATTGGAGGAACT | |
| | TTGCCGACATTGACGACAGCTGGAAGAGCATCAAGAGCATCCTGGACTGGACCTCATTCAATCAGGAGAGGATTGTGGATGTG | |
| | GCAGGACCAGGGGGCTGGAATGATCCTGACATGCTGGTGATTGGCAACTTTGGACTGAGCTGGAATCAGCAGGTCACCCAGAT | |
| | GGCACTGTGGGCTATCATGGCCGCCCCCCTGTTTATGAGCAATGACCTCAGGCACATCTCACCACAAGCCAAGGCACTGCTCCAG | |
| | GATAAGGATGTCATTGCAATTAATCAGGATCCACTGGGCAAACAAGGCTATCAGCTGAGACAGGGAGATAATTTTGAGGTGTG | |
| | GGAGAGGCCTCTGTCTGGCCTGGCATGGGCCGTGGCTATGATCAACAGACAGGAGATCGGCGGCCCAAGATCATACACAATCG | |
| | CCGTGGCATCACTGGGCAAGGGAGTGGCCTGCAACCCTGCCTGCTTTATCACTCAGCTGCTGCCTGTGAAAAGGAAGCTGGGCT | |
| | TCTATGAGTGGACAAGCAGACTGAGGAGCCACATCAACCCCACAGGAACAGTCCTGCTCCAACTGGAAAATACTATGCAAATGT | |
| | 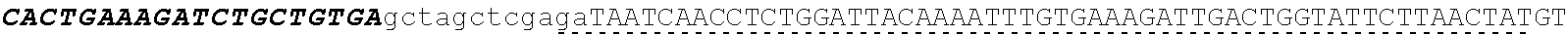 | |
| | 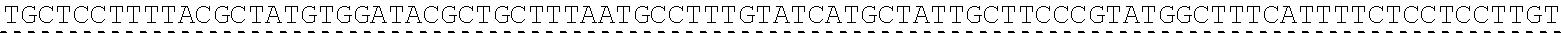 | |
| | 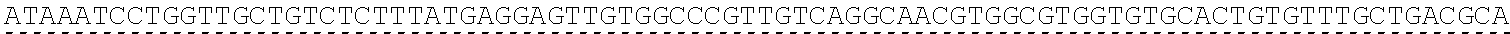 | |
| | 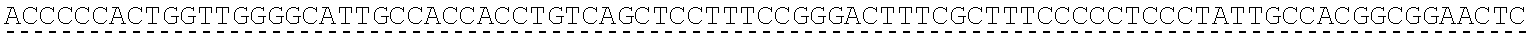 | |
| | 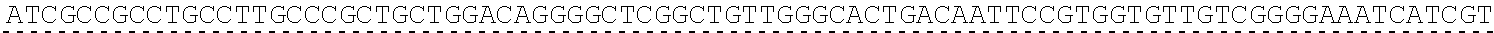 | |
| | 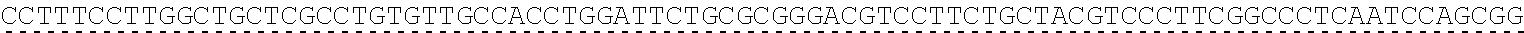 | |
| | 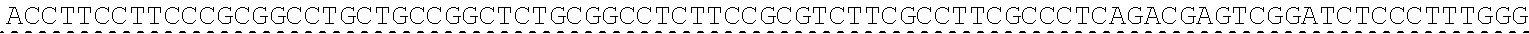 | |
| | 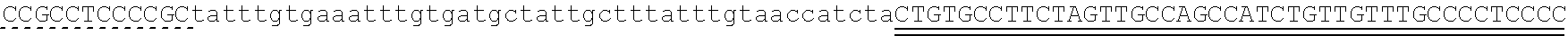 | |
| | CGTGCCTTCCTTGACCCTGGAAGGTGCCACTCCCACTGTCCTTTCCTAATAAAATGAGGAAATTGCATCGCATTGTCTGAGTAGGTG | |
| | TCATTCTATTCTGGGGGGTGGGGTGGGGCAGGACAGCAAGGGGGAGGATTGGGAAGACAATAGCAGGCATGCTGGGGATGCG | |
| | 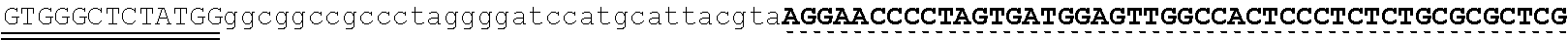 | |
| | 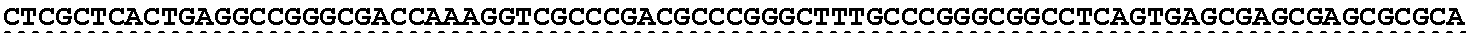 | |
| | 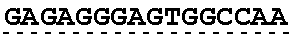 | |
| Construct | 5′ITR (SEQ ID NO: 17), Apo E/C-I (SEQ ID NO: 19), A1AT (SEQ ID NO: 20), | |
| 10A | β-globin intron (SEQ ID NO: 21), KOZAK 40 consensus leader sequence (SEQ ID NO: 22), | |
| | IgG1 coC-CpG signal peptide (SEQ ID 14), coB-CpG mature peptide (SEQ | |
| | 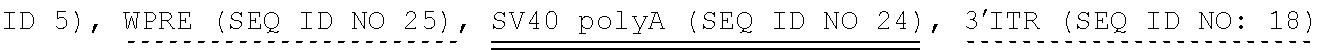 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatcaAGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAGctcctgggcaacctgctggtctctctgctggcccatcacttt | |
| | ggcaaagcacgcgtcGCCGCCACCATGGGGTGGTCATGTATTATCCTGTTTCTGGTGGCCACAGCCACAGGCGTGCACAGCCTGGACA | |
| | ATGGGCTGGCCAGGACTCCAACCATGGGCTGGCTCCACTGGGAGAGGTTCATGTGTAATCTGGACTGCCAAGAAGAACCAGAT | |
| | AGCTGTATCTCTGAGAAACTGTTTATGGAAATGGCTGAGCTCATGGTCAGCGAGGGCTGGAAGGATGCCGGCTACGAGTATCT | |
| | GTGTATCGATGACTGCTGGATGGCCCCTCAGAGGGATTCAGAGGGCAGGCTCCAGGCTGACCCACAGAGGTTTCCCCATGGCAT | |
| | CAGGCAGCTGGCCAATTACGTGCACAGCAAAGGCCTGAAGCTGGGGATCTATGCCGATGTGGGGAACAAGACCTGTGCCGGAT | |
| | TTCCAGGCAGCTTTGGCTACTATGACATCGATGCCCAGACCTTTGCCGATTGGGGGGTGGATCTCCTGAAATTTGATGGATGCTA | |
| | TTGTGACAGCCTGGAGAACCTGGCCGATGGCTACAAACACATGAGCCTGGCCCTGAACAGGACCGGCAGGAGCATCGTGTACA | |
| | GCTGTGAGTGGCCTCTGTACATGTGGCCCTTCCAGAAGCCAAATTACACAGAGATTAGACAGTATTGCAATCATTGGAGGAACT | |
| | TTGCCGACATTGACGACAGCTGGAAGAGCATCAAGAGCATCCTGGACTGGACCTCATTCAATCAGGAGAGGATTGTGGATGTG | |
| | GCAGGACCAGGGGGCTGGAATGATCCTGACATGCTGGTGATTGGCAACTTTGGACTGAGCTGGAATCAGCAGGTCACCCAGAT | |
| | GGCACTGTGGGCTATCATGGCCGCCCCCCTGTTTATGAGCAATGACCTCAGGCACATCTCACCACAAGCCAAGGCACTGCTCCAG | |
| | GATAAGGATGTCATTGCAATTAATCAGGATCCACTGGGCAAACAAGGCTATCAGCTGAGACAGGGAGATAATTTTGAGGTGTG | |
| | GGAGAGGCCTCTGTCTGGCCTGGCATGGGCCGTGGCTATGATCAACAGACAGGAGATCGGCGGCCCAAGATCATACACAATCG | |
| | CCGTGGCATCACTGGGCAAGGGAGTGGCCTGCAACCCTGCCTGCTTTATCACTCAGCTGCTGCCTGTGAAAAGGAAGCTGGGCT | |
| | TCTATGAGTGGACAAGCAGACTGAGGAGCCACATCAACCCCACAGGAACAGTCCTGCTCCAACTGGAAAATACTATGCAAATGT | |
| | 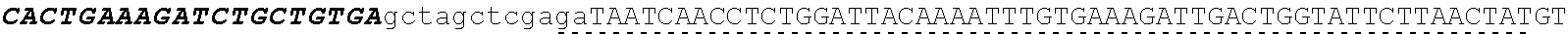 | |
| | 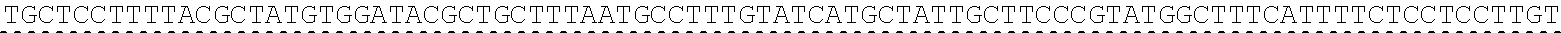 | |
| | 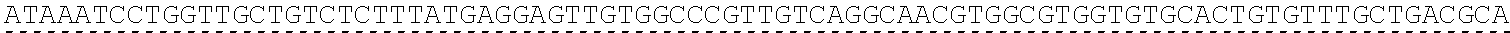 | |
| | 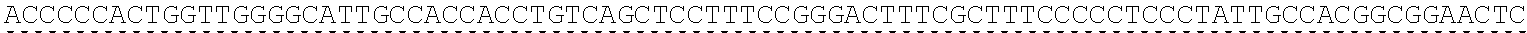 | |
| | 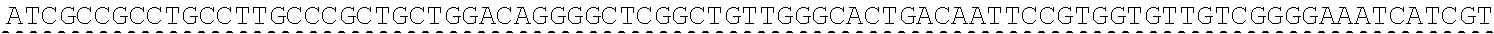 | |
| | 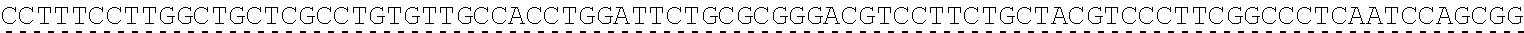 | |
| | 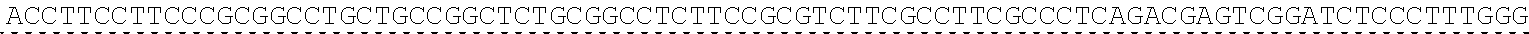 | |
| | 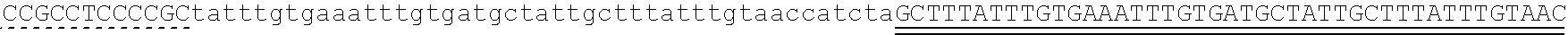 | |
| | 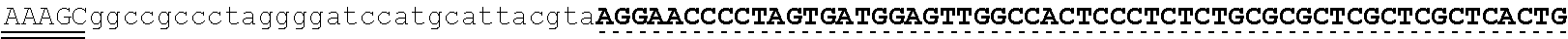 | |
| | 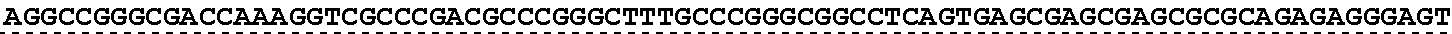 | |
| | 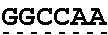 | |
| Construct | 5′ITR (SEQ ID NO: 17), Apo E/C-I (SEQ ID NO: 19), A1AT (SEQ ID NO: 20), | 41 |
| 11A | β-globin intron (SEQ ID NO: 21), KOZAK consensus leader sequence (SEQ ID NO: 22), | |
| | IgG1 coC-CpG signal peptide (SEQ ID 14), coB-CpG mature peptide (SEQ | |
| | 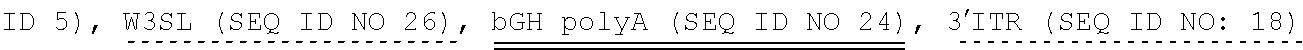 | |
| | TTGGCCACTCCCTCTCTGCGCGCTCGCTCGCTCACTGAGGCCGCCCGGGCAAAGCCCGGGCGTCGGGCGACCTTTGGTCGCCCGG | |
| | CCTCAGTGAGCGAGCGAGCGCGCAGAGAGGGAGTGGCCAACTCCATCACTAGGGGTTCCTatgcattacgtaggacgtcccctgcaggca | |
| | gtgtagttaatgattaacccgccatgctacttatctacgtagccatgctctagaggatcaAGGCTCAGAGGCACACAGGAGTTTCTGGGCTCACCCTGCC | |
| | CCCTTCCAACCCCTCAGTTCCCATCCTCCAGCAGCTGTTTGTGTGCTGCCTCTGAAGTCCACACTGAACAAACTTCAGCCTACTCATG | |
| | TCCCTAAAATGGGCAAACATTGCAAGCAGCAAACAGCAAACACACAGCCCTCCCTGCCTGCTGACCTTGGAGCTGGGGCAGAGGT | |
| | CAGAGACCTCTCTGGGCCCATGCCACCTCCAACATCCACTCGACCCCTTGGAATTTCGGTGGAGAGGAGCAGAGGTTGTCCTGGC | |
| | GTGGTTTAGGTAGTGTGAGAGGGgtacccgggGATCTTGCTACCAGTGGAACAGCCACTAAGGATTCTGCAGTGAGAGCAGAGGGC | |
| | CAGCTAAGTGGTACTCTCCCAGAGACTGTCTGACTCACGCCACCCCCTCCACCTTGGACACAGGACGCTGTGGTTTCTGAGCCAGGT | |
| | ACAATGACTCCTTTCGGTAAGTGCAGTGGAAGCTGTACACTGCCCAGGCAAAGCGTCCGGGCAGCGTAGGCGGGCGACTCAGATC | |
| | CCAGCCAGTGGACTTAGCCCCTGTTTGCTCCTCCGATAACTGGGGTGACCTTGGTTAATATTCACCAGCAGCCTCCCCCGTTGCCCCT | |
| | CTGGATCCACTGCTTAAATACGGACGAGGACAGGGCCCTGTCTCCTCAGCTTCAGGCACCACCACTGACCTGGGACAGTgaatagatc | |
| | ctgagaacttcaggGTGAGTCTATGGGACCCTTGATGTTTTCTTTCCCCTTCTTTTCTATGGTTAAGTTCATGTCATAGGAAGGGGAGA | |
| | AGTAACAGGGTACACATATTGACCAAATCAGGGTAATTTTGCATTTGTAATTTTAAAAAATGCTTTCTTCTTTTAATATACTTTTT | |
| | TGTTTATCTTATTTCTAATACTTTCCCTAATCTCTTTCTTTCAGGGCAATAATGATACAATGTATCATGCCTCTTTGCACCATTCTAA | |
| | AGAATAACAGTGATAATTTCTGGGTTAAGGCAATAGCAATATTTCTGCATATAAATATTTCTGCATATAAATTGTAACTGATGTA | |
| | AGAGGTTTCATATTGCTAATAGCAGCTACAATCCAGCTACCATTCTGCTTTTATTTTCTGGTTGGGATAAGGCTGGATTATTCTGA | |
| | GTCCAAGCTAGGCCCTTTTGCTAATCTTGTTCATACCTCTTATCTTCCTCCCACAGctcctgggcaacctgctggtctctctgctggcccatcacttt | |
| | ggcaaagcacgcgtcGCCGCCACCATGGGGTGGTCATGTATTATCCTGTTTCTGGTGGCCACAGCCACAGGCGTGCACAGCCTGGACA | |
| | ATGGGCTGGCCAGGACTCCAACCATGGGCTGGCTCCACTGGGAGAGGTTCATGTGTAATCTGGACTGCCAAGAAGAACCAGAT | |
| | AGCTGTATCTCTGAGAAACTGTTTATGGAAATGGCTGAGCTCATGGTCAGCGAGGGCTGGAAGGATGCCGGCTACGAGTATCT | |
| | GTGTATCGATGACTGCTGGATGGCCCCTCAGAGGGATTCAGAGGGCAGGCTCCAGGCTGACCCACAGAGGTTTCCCCATGGCAT | |
| | CAGGCAGCTGGCCAATTACGTGCACAGCAAAGGCCTGAAGCTGGGGATCTATGCCGATGTGGGGAACAAGACCTGTGCCGGAT | |
| | TTCCAGGCAGCTTTGGCTACTATGACATCGATGCCCAGACCTTTGCCGATTGGGGGGTGGATCTCCTGAAATTTGATGGATGCTA | |
| | TTGTGACAGCCTGGAGAACCTGGCCGATGGCTACAAACACATGAGCCTGGCCCTGAACAGGACCGGCAGGAGCATCGTGTACA | |
| | GCTGTGAGTGGCCTCTGTACATGTGGCCCTTCCAGAAGCCAAATTACACAGAGATTAGACAGTATTGCAATCATTGGAGGAACT | |
| | TTGCCGACATTGACGACAGCTGGAAGAGCATCAAGAGCATCCTGGACTGGACCTCATTCAATCAGGAGAGGATTGTGGATGTG | |
| | GCAGGACCAGGGGGCTGGAATGATCCTGACATGCTGGTGATTGGCAACTTTGGACTGAGCTGGAATCAGCAGGTCACCCAGAT | |
| | GGCACTGTGGGCTATCATGGCCGCCCCCCTGTTTATGAGCAATGACCTCAGGCACATCTCACCACAAGCCAAGGCACTGCTCCAG | |
| | GATAAGGATGTCATTGCAATTAATCAGGATCCACTGGGCAAACAAGGCTATCAGCTGAGACAGGGAGATAATTTTGAGGTGTG | |
| | GGAGAGGCCTCTGTCTGGCCTGGCATGGGCCGTGGCTATGATCAACAGACAGGAGATCGGCGGCCCAAGATCATACACAATCG | |
| | CCGTGGCATCACTGGGCAAGGGAGTGGCCTGCAACCCTGCCTGCTTTATCACTCAGCTGCTGCCTGTGAAAAGGAAGCTGGGCT | |
| | TCTATGAGTGGACAAGCAGACTGAGGAGCCACATCAACCCCACAGGAACAGTCCTGCTCCAACTGGAAAATACTATGCAAATGT | |
| | 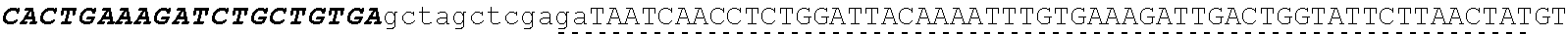 | |
| | 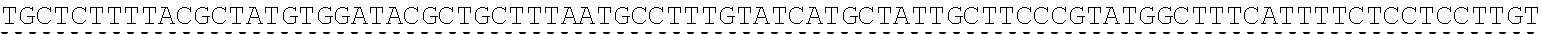 | |
| | 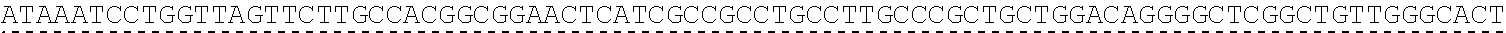 | |
| | 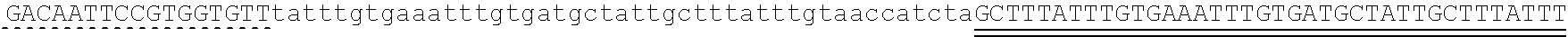 | |
| | 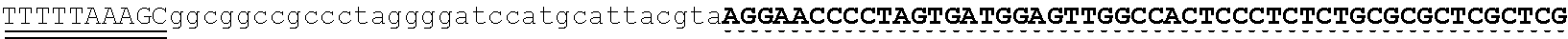 | |
| | 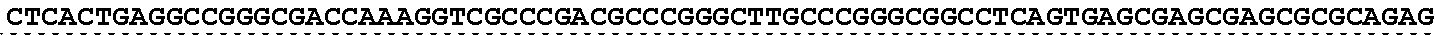 | |
| | 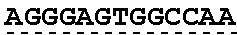 | |
| SL65 | MAADGYLPDWLEDTLSEGIREWWALKPGAPQPKANQQHQDNGRGLVLPGYKYLGPFNGLDKGEPVNEADAAALEHDKAYDKQLEQGDN | 45 |
| | PYLKYNHADAEFQERLQEDTSFGGNLGRAVFQAKKRVLEPLGLVEEGAKTAPGKKRPVEPSPQRSPDSSTGIGKKGQQPARKRLNFGQ | |
| | TGDSESVPDPQPLGEPPAAPSSVGSGTVAAGGGAPMADNNEGADGVGNASGNWHCDSTWLGDRVITTSTRTWALPTYNNHLYKQISSQ | |
| | SGASNDNHYFGYSTPWGYFDFNRFHCHFSPRDWORLINNNWGFRPKRLNFKLFNIQVKEVTTNDGVTTIANNLTSTVQVFSDSEYQLP | |
| | YVLGSAHQGCLPPFPADVFMIPQYGYLTLNNGSQAVGRSSFYCLEYFPSOMLRTGNNFEFSYSFEDVPFHSSYAHSQSLDRLMNPLID | |
| | QYLYYLSRTQSTGGTQGTQQLLFSQAGPANMSAQAKNWLPGPCYRQQRVSTTLSQNNNSNFAWTGATKYHLNGRNSLVNPGVAMATHK | |
| | DDEDRFFPSSGVLIFGKTGATNKTTLENVLMTNEEEIRPTNPVATEEYGIVSSNLQAANTAAQTQVVNNQGALPGMVWQNRDVYLQGP | |
| | IWAKIPHTDGHFHPSPLMGGFGLKNPPPQILIKNTPVPANPPAEFSATKFASFITQYSTGQVSVEIEWELQKENSKRWNPEVQYTSNY | |
| | AKSANVDFTVDNNGLYTEPRPIGTRYLTRPL* | |
| SL65 | ATGGCTGCCGATGGTTATCTTCCAGATTGGCTCGAGGACACTCTCTCTGAAGGCATTCGCGAGTGGTGGGCGCTGAAACCTGGAGCTC | 46 |
| coding | CACAACCCAAGGCCAACCAACAGCATCAGGACAACGGCAGGGGTCTTGTGCTTCCTGGGTACAAGTACCTCGGACCCTTCAACGGACT | |
| sequence | CGACAAGGGAGAGCCGGTCAACGAGGCAGACGCCGCGGCCCTCGAGCACGACAAGGCCTACGACAAGCAGCTCGAGCAGGGGGACAAC | |
| | CCGTACCTCAAGTACAACCACGCCGACGCCGAGTTTCAGGAGCGTCTGCAAGAAGATACGTCTTTTGGGGGCAACCTCGGGCGAGCAG | |
| | TCTTCCAGGCCAAGAAGCGGGTTCTCGAACCTCTCGGTCTGGTTGAGGAAGGCGCTAAGACGGCTCCTGGAAAGAAGAGACCGGTAGA | |
| | GCCGTCACCTCAGCGTTCCCCCGACTCCTCCACGGGCATCGGCAAGAAAGGCCAGCAGCCCGCCAGAAAGAGACTCAATTTCGGTCAG | |
| | ACTGGCGACTCAGAGTCAGTCCCCGACCCTCAACCTCTCGGAGAACCTCCAGCAGCGCCCTCTAGTGTGGGATCTGGTACAGTGGCTG | |
| | CAGGCGGTGGCGCACCAATGGCAGACAATAACGAAGGTGCCGACGGAGTGGGTAATGCCTCAGGAAATTGGCATTGCGATTCCACATG | |
| | GCTGGGCGACAGAGTCATTACCACCAGCACCCGAACCTGGGCCCTGCCCACCTACAACAACCACCTCTACAAGCAAATCTCCAGCCAA | |
| | TCAGGAGCTTCAAACGACAACCACTACTTTGGCTACAGCACCCCTTGGGGGTATTTTGACTTTAACAGATTCCACTGCCACTTCTCAC | |
| | CACGTGACTGGCAGCGACTCATTAACAACAACTGGGGATTCCGGCCCAAGAGACTCAACTTCAAGCTCTTCAACATCCAAGTCAAGGA | |
| | GGTCACGACGAATGATGGCGTCACGACCATCGCTAATAACCTTACCAGCACGGTTCAAGTCTTCTCGGACTCGGAGTACCAGTTGCCG | |
| | TACGTCCTCGGCTCTGCGCACCAGGGCTGCCTCCCTCCGTTCCCGGCGGACGTGTTCATGATTCCCCAGTACGGCTACCTAACACTCA | |
| | ACAACGGTAGTCAGGCCGTGGGACGCTCCTCCTTTTACTGCCTGGAATATTTCCCATCGCAGATGCTGAGAACGGGCAATAACTTTGA | |
| | GTTCAGCTACAGCTTCGAGGACGTGCCTTTCCACAGCAGCTACGCACACAGCCAGAGCTTGGACCGACTGATGAATCCTCTCATTGAC | |
| | CAGTACCTGTACTACTTATCCAGAACTCAGTCCACAGGAGGAACTCAAGGTACCCAGCAATTGTTATTTTCTCAAGCTGGGCCTGCAA | |
| | ACATGTCGGCTCAGGCCAAGAACTGGCTGCCTGGACCTTGCTACCGGCAGCAGCGAGTCTCCACGACACTGTCGCAAAACAACAACAG | |
| | CAACTTTGCTTGGACTGGTGCCACCAAATATCACCTGAACGGCAGAAACTCGTTGGTTAATCCCGGCGTCGCCATGGCAACTCACAAG | |
| | GACGACGAGGACCGCTTTTTCCCATCCAGCGGAGTCCTGATTTTTGGAAAAACTGGAGCAACTAACAAAACTACATTGGAAAATGTGT | |
| | TAATGACAAATGAAGAAGAAATTCGTCCTACTAATCCTGTAGCCACGGAAGAATACGGGATAGTCAGCAGCAACTTACAAGCGGCTAA | |
| | TACTGCAGCCCAGACACAAGTIGTCAACAACCAGGGAGCCTTACCTGGCATGGTCTGGCAGAACCGGGACGTGTACCTGCAGGGTCCC | |
| | ATTTGGGCCAAAATTCCTCACACAGATGGACACTTTCACCCGTCTCCTCTTATGGGCGGCTTTGGACTCAAGAACCCGCCTCCTCAGA | |
| | TCCTCATCAAAAACACGCCTGTTCCTGCGAATCCTCCGGCGGAGTTTTCAGCTACAAAGTTTGCTTCATTCATCACCCAGTATTCCAC | |
| | AGGACAAGTGAGCGTGGAGATTGAATGGGAGCTGCAGAAAGAAAACAGCAAACGCTGGAATCCCGAAGTGCAGTATACATCTAACTAT | |
| | GCAAAATCTGCCAACGTTGATTTCACTGTGGACAACAATGGACTTTATACTGAGCCTCGCCCCATTGGCACCCGTTACCTTACCCGTC | |
| | CCCTGTAA | |
| Construct | 5′ITR (SEQ ID NO: 17), Apo E/C-I (SEQ ID NO: 19), A1AT (SEQ ID NO: 20), β-globin intron | 51 |
| 1B | (SEQ ID NO: 21), KOZAK consensus leader sequence (SEQ ID NO: 22), hGLA signal peptide | |
| | 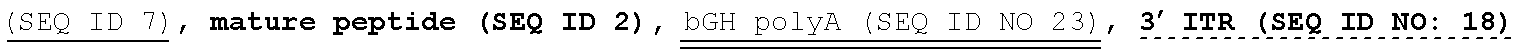 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGCAGCTGAGGAACCCAGAACTACATCTGGGCTGCGCGCTTGCGCTTCGCTTCCTGGCCCTCGTTT | |
| | CCTGGGACATCCCTGGGGCTAGAGCACTGGACAATGGATTGGCAAGGACGCCTACCATGGGCTGGCTGCACTGGGAGCGCTTCATGTG | |
| | CAACCTTGACTGCCAGGAAGAGCCAGATTCCTGCATCAGTGAGAAGCTCTTCATGGAGATGGCAGAGCTCATGGTCTCAGAAGGCTGG | |
| | AAGGATGCAGGTTATGAGTACCTCTGCATTGATGACTGTTGGATGGCTCCCCAAAGAGATTCAGAAGGCAGACTTCAGGCAGACCCTC | |
| | AGCGCTTTCCTCATGGGATTCGCCAGCTAGCTAATTATGTTCACAGCAAAGGACTGAAGCTAGGGATTTATGCAGATGTTGGAAATAA | |
| | AACCTGCGCAGGCTTCCCTGGGAGTTTTGGATACTACGACATTGATGCCCAGACCTTTGCTGACTGGGGAGTAGATCTGCTAAAATTT | |
| | GATGGTTGTTACTGTGACAGTTTGGAAAATTTGGCAGATGGTTATAAGCACATGTCCTTGGCCCTGAATAGGACTGGCAGAAGCATTG | |
| | TGTACTCCTGTGAGTGGCCTCTTTATATGTGGCCCTTTCAAAAGCCCAATTATACAGAAATCCGACAGTACTGCAATCACTGGCGAAA | |
| | TTTTGCTGACATTGATGATTCCTGGAAAAGTATAAAGAGTATCTTGGACTGGACATCTTTTAACCAGGAGAGAATTGTTGATGTTGCT | |
| | GGACCAGGGGGTTGGAATGACCCAGATATGTTAGTGATTGGCAACTTTGGCCTCAGCTGGAATCAGCAAGTAACTCAGATGGCCCTCT | |
| | GGGCTATCATGGCTGCTCCTTTATTCATGTCTAATGACCTCCGACACATCAGCCCTCAAGCCAAAGCTCTCCTTCAGGATAAGGACGT | |
| | AATTGCCATCAATCAGGACCCCTTGGGCAAGCAAGGGTACCAGCTTAGACAGGGAGACAACTTTGAAGTGTGGGAACGACCTCTCTCA | |
| | GGCTTAGCCTGGGCTGTAGCTATGATAAACCGGCAGGAGATTGGTGGACCTCGCTCTTATACCATCGCAGTTGCTTCCCTGGGTAAAG | |
| | GAGTGGCCTGTAATCCTGCCTGCTTCATCACACAGCTCCTCCCTGTGAAAAGGAAGCTAGGGTTCTATGAATGGACTTCAAGGTTAAG | |
| | AAGTCACATAAATCCCACAGGCACTGTTTTGCTTCAGCTAGAAAATACAATGCAGATGTCATTAAAAGACTTACTTTAAgctagctcg | |
| | agcgaCTGTGCCTTCTAGTTGCCAGCCATCTGTTGTTTGCCCCTCCCCCGTGCCTTCCTTGACCCTGGAAGGTGCCACTCCCACTGTC | |
| | CTTTCCTAATAAAATGAGGAAATTGCATCGCATTGTCTGAGTAGGTGTCATTCTATTCTGGGGGGGGGGTGGGGCAGGACAGCAAGG | |
| | GGGAGGATTGGGAAGACAATAGCAGGCATGCTGGGGATGCGGTGGGCTCTATGGgttaatcattaactacagcggccgccctagggga | |
| | 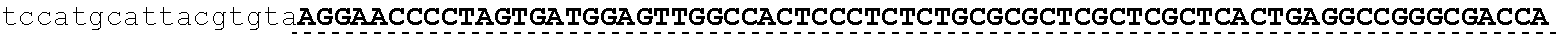 | |
| | 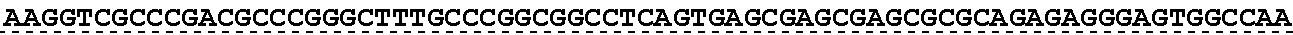 | |
| Construct | 5′ITR (SEQ ID NO: 17), Apo E/C-I (SEQ ID NO: 19), A1AT (SEQ ID NO: 20), β-globin intron | 52 |
| 2B | (SEQ ID NO: 21), KOZAK consensus leader sequence (SEQ ID NO: 22), GLA coA signal | |
| | 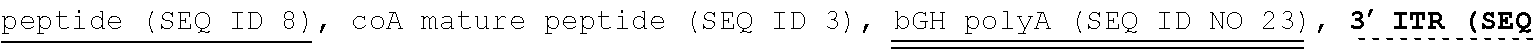 | |
| | 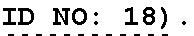 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGCAGCTGAGAAATCCTGAGCTGCACCTGGGCTGTGCCCTGGCTCTGAGATTTCTGGCTCTGGTGT | |
| | CCTGGGACATCCCTGGCGCTAGAGCCCTGGATAATGGCCTGGCCAGAACACCTACAATGGGCTGGCTGCACTGGGAGAGATTCATGTG | |
| | CAACCTGGACTGCCAAGAGGAACCCGACAGCTGCATCAGCGAGAAGCTGTTCATGGAAATGGCCGAGCTGATGGTGTCCGAAGGCTGG | |
| | AAGGATGCCGGCTACGAGTACCTGTGCATCGACGACTGTTGGATGGCCCCTCAGAGAGACTCTGAGGGCAGACTGCAGGCCGATCCTC | |
| | AGAGATTTCCCCACGGCATTAGACAGCTGGCCAACTACGTGCACAGCAAGGGCCTGAAGCTGGGCATCTATGCCGACGTGGGCAACAA | |
| | GACCTGTGCCGGCTTTCCTGGCAGCTTCGGCTACTACGATATCGACGCCCAGACCTTCGCCGATTGGGGAGTCGATCTGCTGAAGTTC | |
| | GACGGCTGCTACTGCGACAGCCTGGAAAATCTGGCCGACGGCTACAAGCACATGTCTCTGGCCCTGAATCGGACCGGCAGATCCATCG | |
| | TGTACAGCTGCGAGTGGCCCCTGTACATGTGGCCCTTCCAGAAGCCTAACTACACCGAGATCAGACAGTACTGCAACCACTGGCGGAA | |
| | CTTCGCCGACATCGACGATAGCTGGAAGTCCATCAAGAGCATCCTGGACTGGACCAGCTTCAATCAAGAGCGGATCGTGGACGTGGCA | |
| | GGACCTGGCGGATGGAACGATCCTGACATGCTGGTCATCGGCAACTTCGGCCTGAGCTGGAACCAGCAAGTGACCCAGATGGCCCTGT | |
| | GGGCCATTATGGCCGCTCCTCTGTTCATGAGCAACGACCTGAGACACATCAGCCCTCAGGCCAAGGCTCTGCTGCAGGACAAGGATGT | |
| | GATCGCTATCAACCAGGATCCTCTGGGCAAGCAGGGCTACCAGCTGAGACAGGGCGACAATTTCGAAGTGTGGGAAAGACCCCTGAGC | |
| | GGACTGGCTTGGGCCGTCGCCATGATCAACAGACAAGAGATCGGCGGACCCCGGTCCTACACAATTGCCGTGGCTTCTCTCGGCAAAG | |
| | GCGTGGCCTGTAATCCCGCCTGCTTTATCACACAGCTGCTGCCCGTGAAGAGAAAGCTGGGCTTTTACGAGTGGACCAGCAGACTGCG | |
| | GAGCCACATCAATCCTACCGGCACAGTGCTGCTGCAGCTGGAAAACACAATGCAGATGAGCCTGAAGGACCTGCTGTGAgctagctcg | |
| | agcgaCTGTGCCTTCTAGTTGCCAGCCATCTGTTGTTTGCCCCTCCCCCGTGCCTTCCTTGACCCTGGAAGGTGCCACTCCCACTGTC | |
| | CTTTCCTAATAAAATGAGGAAATTGCATCGCATTGTCTGAGTAGGTGTCATTCTATTCTGGGGGGTGGGGTGGGGCAGGACAGCAAGG | |
| | GGGAGGATTGGGAAGACAATAGCAGGCATGCTGGGGATGCGGTGGGCTCTATGGgttaatcattaactacagcggccgccctagggga | |
| | 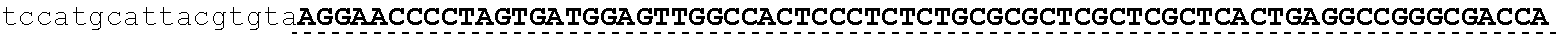 | |
| | 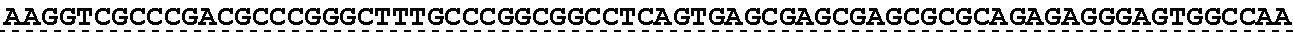 | |
| Construct | 5′ITR (SEQ ID NO: 17), Apo E/C-I (SEQ ID NO: 19), A1AT (SEQ ID NO: 20), β-globin intron | 53 |
| 3B | (SEQ ID NO: 21), KOZAK consensus leader sequence (SEQ ID NO: 22), GLA coB signal | |
| | 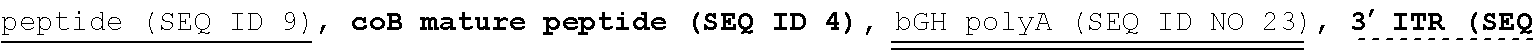 | |
| | 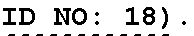 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGCAGCTCAGAAACCCAGAACTCCATCTGGGGTGCGCTCTCGCCCTCCGCTTCCTGGCACTCGTCA | |
| | GCTGGGATATTCCTGGGGCTAGAGCCCTCGATAATGGGCTCGCTCGGACTCCAACCATGGGCTGGCTCCACTGGGAACGCTTCATGTG | |
| | TAATCTGGACTGCCAAGAAGAACCAGATAGCTGTATCAGCGAAAAACTGTTTATGGAAATGGCTGAGCTCATGGTCAGCGAGGGCTGG | |
| | AAGGACGCCGGCTACGAGTATCTGTGTATCGACGACTGCTGGATGGCCCCTCAGCGGGATTCAGAGGGCAGGCTCCAGGCTGACCCAC | |
| | AGCGGTTTCCCCACGGAATTCGGCAGCTGGCCAATTACGTGCACAGCAAAGGCCTGAAGCTGGGGATCTACGCTGATGTGGGGAACAA | |
| | GACCTGCGCTGGATTTCCAGGCAGCTTCGGCTACTACGATATCGACGCACAGACCTTCGCCGATTGGGGCGTCGACCTCCTGAAATTT | |
| | GATGGATGCTATTGCGATAGCCTCGAAAACCTGGCCGACGGGTACAAACACATGAGCCTCGCTCTGAACCGGACCGGCCGGAGCATCG | |
| | TGTACAGCTGCGAATGGCCTCTGTACATGTGGCCCTTCCAGAAGCCAAATTACACAGAGATTAGACAGTATTGCAATCATTGGCGGAA | |
| | CTTCGCCGACATCGATGACAGCTGGAAGAGCATCAAGAGCATCCTCGACTGGACCTCATTCAATCAGGAACGCATTGTGGACGTGGCA | |
| | GGACCAGGCGGGTGGAATGATCCCGACATGCTGGTGATTGGCAACTTTGGACTGAGCTGGAATCAGCAGGTCACCCAGATGGCACTGT | |
| | GGGCTATCATGGCCGCCCCCCTGTTTATGAGCAACGACCTCAGGCACATCTCACCACAAGCCAAGGCACTGCTCCAGGATAAGGATGT | |
| | CATTGCAATTAATCAGGATCCACTGGGCAAACAAGGCTATCAGCTGAGACAGGGAGATAATTTCGAAGTGTGGGAGCGGCCTCTGAGC | |
| | GGACTGGCATGGGCCGTGGCTATGATCAACAGACAGGAGATCGGCGGCCCAAGATCATACACAATCGCCGTGGCATCACTGGGCAAGG | |
| | GAGTCGCCTGCAACCCCGCATGCTTTATCACTCAGCTGCTGCCTGTGAAAAGGAAGCTGGGCTTCTACGAATGGACAAGCAGACTGAG | |
| | GAGCCACATCAACCCCACAGGAACAGTCCTGCTCCAACTGGAAAATACTATGCAAATGTCACTGAAAGATCTGCTGTGAgctagctcg | |
| | agcgaCTGTGCCTTCTAGTTGCCAGCCATCTGTTGTTTGCCCCTCCCCCGTGCCTTCCTTGACCCTGGAAGGTGCCACTCCCACTGTC | |
| | CTTTCCTAATAAAATGAGGAAATTGCATCGCATTGTCTGAGTAGGTGTCATTCTATTCTGGGGGGTGGGGTGGGGCAGGACAGCAAGG | |
| | GGGAGGATTGGGAAGACAATAGCAGGCATGCTGGGGATGCGGTGGGCTCTATGGgttaatcattaactacagcggccgccctagggga | |
| | 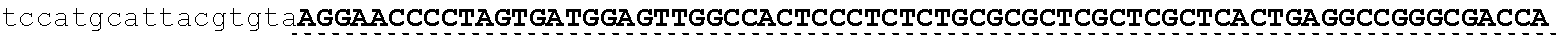 | |
| | 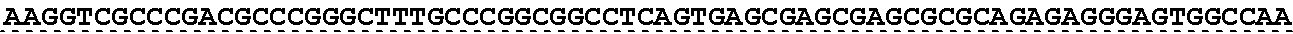 | |
| Construct | 5′ITR (SEQ ID NO: 17), Apo E/C-I (SEQ ID NO: 19), A1AT (SEQ ID NO: 20), β-globin intron | 54 |
| 4B | (SEQ ID NO: 21) , KOZAK consensus leader sequence (SEQ ID NO: 22), GLA coB-CpG signal | |
| | 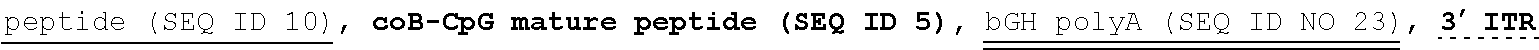 | |
| | 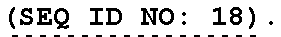 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGCAGCTCAGAAACCCAGAACTCCATCTGGGGTGTGCCCTGGCCCTGAGGTTCCTGGCACTGGTGA | |
| | GCTGGGATATTCCTGGGGCTAGAGCCCTGGACAATGGGCTGGCCAGGACTCCAACCATGGGCTGGCTCCACTGGGAGAGGTTCATGTG | |
| | TAATCTGGACTGCCAAGAAGAACCAGATAGCTGTATCTCTGAGAAACTGTTTATGGAAATGGCTGAGCTCATGGTCAGCGAGGGCTGG | |
| | AAGGATGCCGGCTACGAGTATCTGTGTATCGATGACTGCTGGATGGCCCCTCAGAGGGATTCAGAGGGCAGGCTCCAGGCTGACCCAC | |
| | AGAGGTTTCCCCATGGCATCAGGCAGCTGGCCAATTACGTGCACAGCAAAGGCCTGAAGCTGGGGATCTATGCCGATGTGGGGAACAA | |
| | GACCTGTGCCGGATTTCCAGGCAGCTTTGGCTACTATGACATCGATGCCCAGACCTTTGCCGATTGGGGGGTGGATCTCCTGAAATTT | |
| | GATGGATGCTATTGTGACAGCCTGGAGAACCTGGCCGATGGCTACAAACACATGAGCCTGGCCCTGAACAGGACCGGCAGGAGCATCG | |
| | TGTACAGCTGTGAGTGGCCTCTGTACATGTGGCCCTTCCAGAAGCCAAATTACACAGAGATTAGACAGTATTGCAATCATTGGAGGAA | |
| | CTTTGCCGACATTGACGACAGCTGGAAGAGCATCAAGAGCATCCTGGACTGGACCTCATTCAATCAGGAGAGGATTGTGGATGTGGCA | |
| | GGACCAGGGGGCTGGAATGATCCTGACATGCTGGTGATTGGCAACTTTGGACTGAGCTGGAATCAGCAGGTCACCCAGATGGCACTGT | |
| | GGGCTATCATGGCCGCCCCCCTGTTTATGAGCAATGACCTCAGGCACATCTCACCACAAGCCAAGGCACTGCTCCAGGATAAGGATGT | |
| | CATTGCAATTAATCAGGATCCACTGGGCAAACAAGGCTATCAGCTGAGACAGGGAGATAATTTTGAGGTGTGGGAGAGGCCTCTGTCT | |
| | GGCCTGGCATGGGCCGTGGCTATGATCAACAGACAGGAGATCGGCGGCCCAAGATCATACACAATCGCCGTGGCATCACTGGGCAAGG | |
| | GAGTGGCCTGCAACCCTGCCTGCTTTATCACTCAGCTGCTGCCTGTGAAAAGGAAGCTGGGCTTCTATGAGTGGACAAGCAGACTGAG | |
| | GAGCCACATCAACCCCACAGGAACAGTCCTGCTCCAACTGGAAAATACTATGCAAATGTCACTGAAAGATCTGCTGTGAgctagctcg | |
| | agcgaCTGTGCCTTCTAGTTGCCAGCCATCTGTTGTTTGCCCCTCCCCCGTGCCTTCCTTGACCCTGGAAGGTGCCACTCCCACTGTC | |
| | CTTTCCTAATAAAATGAGGAAATTGCATCGCATTGTCTGAGTAGGTGTCATTCTATTCTGGGGGGTGGGGGGGGCAGGACAGCAAGG | |
| | GGGAGGATTGGGAAGACAATAGCAGGCATGCTGGGGATGCGGTGGGCTCTATGGgttaatcattaactacagcggccgccctagggga | |
| | 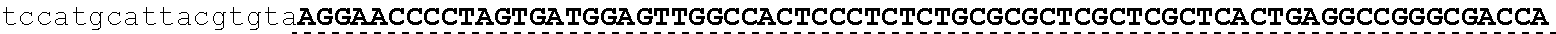 | |
| | 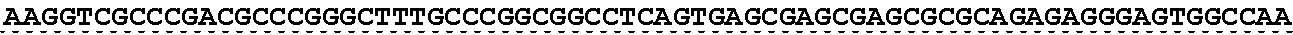 | |
| Construct | 5′ITR (SEQ ID NO: 17), Apo E/C-I (SEQ ID NO: 19), A1AT (SEQ ID NO: 20), β-globin intron | 55 |
| 5B | (SEQ ID NO: 21) , KOZAK consensus leader sequence (SEQ ID NO: 22), IgG1 signal peptide | |
| | 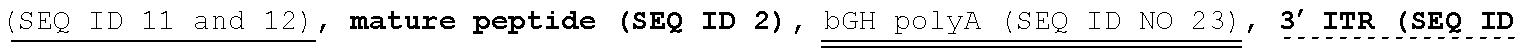 | |
| | 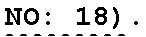 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGGGCTGGAGCTGCATCATCCTGTTCCTGGTGGCCACCGCCACCGGCGTGCACAGCCTGGACAATG | |
| | GATTGGCAAGGACGCCTACCATGGGCTGGCTGCACTGGGAGCGCTTCATGTGCAACCTTGACTGCCAGGAAGAGCCAGATTCCTGCAT | |
| | CAGTGAGAAGCTCTTCATGGAGATGGCAGAGCTCATGGTCTCAGAAGGCTGGAAGGATGCAGGTTATGAGTACCTCTGCATTGATGAC | |
| | TGTTGGATGGCTCCCCAAAGAGATTCAGAAGGCAGACTTCAGGCAGACCCTCAGCGCTTTCCTCATGGGATTCGCCAGCTAGCTAATT | |
| | ATGTTCACAGCAAAGGACTGAAGCTAGGGATTTATGCAGATGTTGGAAATAAAACCTGCGCAGGCTTCCCTGGGAGTTTTGGATACTA | |
| | CGACATTGATGCCCAGACCTTTGCTGACTGGGGAGTAGATCTGCTAAAATTTGATGGTTGTTACTGTGACAGTTTGGAAAATTTGGCA | |
| | GATGGTTATAAGCACATGTCCTTGGCCCTGAATAGGACTGGCAGAAGCATTGTGTACTCCTGTGAGTGGCCTCTTTATATGTGGCCCT | |
| | TTCAAAAGCCCAATTATACAGAAATCCGACAGTACTGCAATCACTGGCGAAATTTTGCTGACATTGATGATTCCTGGAAAAGTATAAA | |
| | GAGTATCTTGGACTGGACATCTTTTAACCAGGAGAGAATTGTTGATGTTGCTGGACCAGGGGGTTGGAATGACCCAGATATGTTAGTG | |
| | ATTGGCAACTTTGGCCTCAGCTGGAATCAGCAAGTAACTCAGATGGCCCTCTGGGCTATCATGGCTGCTCCTTTATTCATGTCTAATG | |
| | ACCTCCGACACATCAGCCCTCAAGCCAAAGCTCTCCTTCAGGATAAGGACGTAATTGCCATCAATCAGGACCCCTTGGGCAAGCAAGG | |
| | GTACCAGCTTAGACAGGGAGACAACTTTGAAGTGTGGGAACGACCTCTCTCAGGCTTAGCCTGGGCTGTAGCTATGATAAACCGGCAG | |
| | GAGATTGGTGGACCTCGCTCTTATACCATCGCAGTTGCTTCCCTGGGTAAAGGAGTGGCCTGTAATCCTGCCTGCTTCATCACACAGC | |
| | TCCTCCCTGTGAAAAGGAAGCTAGGGTTCTATGAATGGACTTCAAGGTTAAGAAGTCACATAAATCCCACAGGCACTGTTTTGCTTCA | |
| | GCTAGAAAATACAATGCAGATGTCATTAAAAGACTTACTTTAAgctagctcgagcgaCTGTGCCTTCTAGTTGCCAGCCATCTGTTGT | |
| | TTGCCCCTCCCCCGTGCCTTCCTTGACCCTGGAAGGTGCCACTCCCACTGTCCTTTCCTAATAAAATGAGGAAATTGCATCGCATTGT | |
| | CTGAGTAGGTGTCATTCTATTCTGGGGGGTGGGGTGGGGCAGGACAGCAAGGGGGAGGATTGGGAAGACAATAGCAGGCATGCTGGGG | |
| | 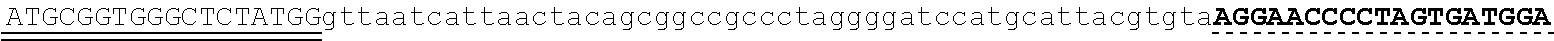 | |
| | 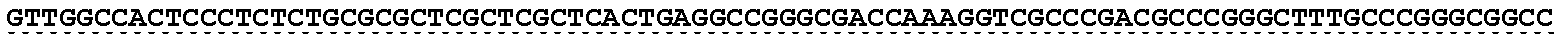 | |
| | 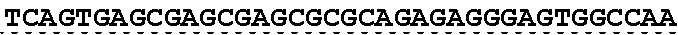 | |
| Construct | 5′ITR (SEQ ID NO: 17), Apo E/C-I (SEQ ID NO: 19), A1AT (SEQ ID NO: 20), β-globin intron | |
| 6B | (SEQ ID NO: 21) , KOZAK consensus leader sequence (SEQ ID NO: 22), IgG1 coC signal | |
| | 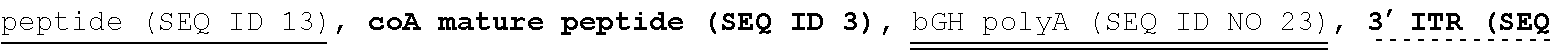 | |
| | 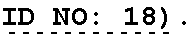 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGGGGTGGTCATGTATTATCCTGTTTCTGGTCGCTACCGCAACCGGAGTGCACAGCCTGGATAATG | |
| | GCCTGGCCAGAACACCTACAATGGGCTGGCTGCACTGGGAGAGATTCATGTGCAACCTGGACTGCCAAGAGGAACCCGACAGCTGCAT | |
| | CAGCGAGAAGCTGTTCATGGAAATGGCCGAGCTGATGGTGTCCGAAGGCTGGAAGGATGCCGGCTACGAGTACCTGTGCATCGACGAC | |
| | TGTTGGATGGCCCCTCAGAGAGACTCTGAGGGCAGACTGCAGGCCGATCCTCAGAGATTTCCCCACGGCATTAGACAGCTGGCCAACT | |
| | ACGTGCACAGCAAGGGCCTGAAGCTGGGCATCTATGCCGACGTGGGCAACAAGACCTGTGCCGGCTTTCCTGGCAGCTTCGGCTACTA | |
| | CGATATCGACGCCCAGACCTTCGCCGATTGGGGAGTCGATCTGCTGAAGTTCGACGGCTGCTACTGCGACAGCCTGGAAAATCTGGCC | |
| | GACGGCTACAAGCACATGTCTCTGGCCCTGAATCGGACCGGCAGATCCATCGTGTACAGCTGCGAGTGGCCCCTGTACATGTGGCCCT | |
| | TCCAGAAGCCTAACTACACCGAGATCAGACAGTACTGCAACCACTGGCGGAACTTCGCCGACATCGACGATAGCTGGAAGTCCATCAA | |
| | GAGCATCCTGGACTGGACCAGCTTCAATCAAGAGCGGATCGTGGACGTGGCAGGACCTGGCGGATGGAACGATCCTGACATGCTGGTC | |
| | ATCGGCAACTTCGGCCTGAGCTGGAACCAGCAAGTGACCCAGATGGCCCTGTGGGCCATTATGGCCGCTCCTCTGTTCATGAGCAACG | |
| | ACCTGAGACACATCAGCCCTCAGGCCAAGGCTCTGCTGCAGGACAAGGATGTGATCGCTATCAACCAGGATCCTCTGGGCAAGCAGGG | |
| | CTACCAGCTGAGACAGGGCGACAATTTCGAAGTGTGGGAAAGACCCCTGAGCGGACTGGCTTGGGCCGTCGCCATGATCAACAGACAA | |
| | GAGATCGGCGGACCCCGGTCCTACACAATTGCCGTGGCTTCTCTCGGCAAAGGCGTGGCCTGTAATCCCGCCTGCTTTATCACACAGC | |
| | TGCTGCCCGTGAAGAGAAAGCTGGGCTTTTACGAGTGGACCAGCAGACTGCGGAGCCACATCAATCCTACCGGCACAGTGCTGCTGCA | |
| | GCTGGAAAACACAATGCAGATGAGCCTGAAGGACCTGCTGTGAgctagctcgagcgaCTGTGCCTTCTAGTTGCCAGCCATCTGTTGT | |
| | TTGCCCCTCCCCCGTGCCTTCCTTGACCCTGGAAGGTGCCACTCCCACTGTCCTTTCCTAATAAAATGAGGAAATTGCATCGCATTGT | |
| | CTGAGTAGGTGTCATTCTATTCTGGGGGGTGGGGTGGGGCAGGACAGCAAGGGGGAGGATTGGGAAGACAATAGCAGGCATGCTGGGG | |
| | 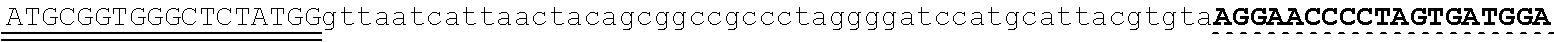 | |
| | 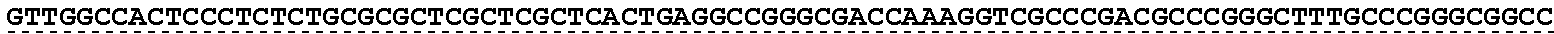 | |
| | 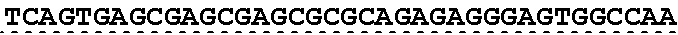 | |
| Construct | 5′ITR (SEQ ID NO: 17), Apo E/C-I (SEQ ID NO: 19), A1AT (SEQ ID NO: 20), β-globin intron | 57 |
| 7B | (SEQ ID NO: 21) , KOZAK consensus leader sequence (SEQ ID NO: 22), IgG1 coC signal | |
| | 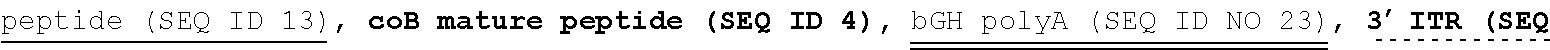 | |
| | 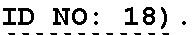 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGGGGTGGTCATGTATTATCCTGTTTCTGGTCGCTACCGCAACCGGAGTGCACAGCCTCGATAATG | |
| | GGCTCGCTCGGACTCCAACCATGGGCTGGCTCCACTGGGAACGCTTCATGTGTAATCTGGACTGCCAAGAAGAACCAGATAGCTGTAT | |
| | CAGCGAAAAACTGTTTATGGAAATGGCTGAGCTCATGGTCAGCGAGGGCTGGAAGGACGCCGGCTACGAGTATCTGTGTATCGACGAC | |
| | TGCTGGATGGCCCCTCAGCGGGATTCAGAGGGCAGGCTCCAGGCTGACCCACAGCGGTTTCCCCACGGAATTCGGCAGCTGGCCAATT | |
| | ACGTGCACAGCAAAGGCCTGAAGCTGGGGATCTACGCTGATGTGGGGAACAAGACCTGCGCTGGATTTCCAGGCAGCTTCGGCTACTA | |
| | CGATATCGACGCACAGACCTTCGCCGATTGGGGCGTCGACCTCCTGAAATTTGATGGATGCTATTGCGATAGCCTCGAAAACCTGGCC | |
| | GACGGGTACAAACACATGAGCCTCGCTCTGAACCGGACCGGCCGGAGCATCGTGTACAGCTGCGAATGGCCTCTGTACATGTGGCCCT | |
| | TCCAGAAGCCAAATTACACAGAGATTAGACAGTATTGCAATCATTGGCGGAACTTCGCCGACATCGATGACAGCTGGAAGAGCATCAA | |
| | GAGCATCCTCGACTGGACCTCATTCAATCAGGAACGCATTGTGGACGTGGCAGGACCAGGCGGGTGGAATGATCCCGACATGCTGGTG | |
| | ATTGGCAACTTTGGACTGAGCTGGAATCAGCAGGTCACCCAGATGGCACTGTGGGCTATCATGGCCGCCCCCCTGTTTATGAGCAACG | |
| | ACCTCAGGCACATCTCACCACAAGCCAAGGCACTGCTCCAGGATAAGGATGTCATTGCAATTAATCAGGATCCACTGGGCAAACAAGG | |
| | CTATCAGCTGAGACAGGGAGATAATTTCGAAGTGTGGGAGCGGCCTCTGAGCGGACTGGCATGGGCCGTGGCTATGATCAACAGACAG | |
| | GAGATCGGCGGCCCAAGATCATACACAATCGCCGTGGCATCACTGGGCAAGGGAGTCGCCTGCAACCCCGCATGCTTTATCACTCAGC | |
| | TGCTGCCTGTGAAAAGGAAGCTGGGCTTCTACGAATGGACAAGCAGACTGAGGAGCCACATCAACCCCACAGGAACAGTCCTGCTCCA | |
| | ACTGGAAAATACTATGCAAATGTCACTGAAAGATCTGCTGTGAgctagctcgagcgaCTGTGCCTTCTAGTTGCCAGCCATCTGTTGT | |
| | TTGCCCCTCCCCCGTGCCTTCCTTGACCCTGGAAGGTGCCACTCCCACTGTCCTTTCCTAATAAAATGAGGAAATTGCATCGCATTGT | |
| | CTGAGTAGGTGTCATTCTATTCTGGGGGGTGGGGTGGGGCAGGACAGCAAGGGGGAGGATTGGGAAGACAATAGCAGGCATGCTGGGG | |
| | 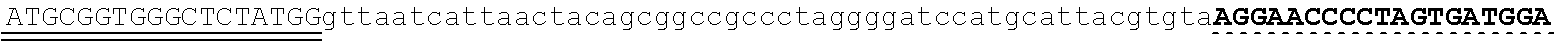 | |
| | 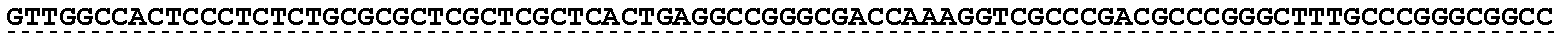 | |
| | 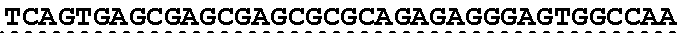 | |
| Construct | 5′ITR (SEQ ID NO: 17), Apo E/C-I (SEQ ID NO: 19), A1AT (SEQ ID NO: 20), β-globin intron | 58 |
| 8B | (SEQ ID NO: 21) , KOZAK consensus leader sequence (SEQ ID NO: 22), IgG1 coC-CpG signal | |
| | 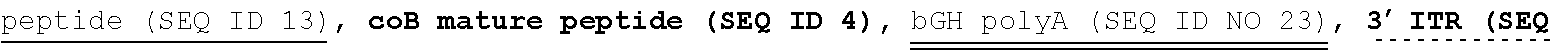 | |
| | 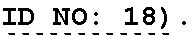 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGGGGTGGTCATGTATTATCCTGTTTCTGGTGGCCACAGCCACAGGCGTGCACAGCCTGGACAATG | |
| | GGCTGGCCAGGACTCCAACCATGGGCTGGCTCCACTGGGAGAGGTTCATGTGTAATCTGGACTGCCAAGAAGAACCAGATAGCTGTAT | |
| | CTCTGAGAAACTGTTTATGGAAATGGCTGAGCTCATGGTCAGCGAGGGCTGGAAGGATGCCGGCTACGAGTATCTGTGTATCGATGAC | |
| | TGCTGGATGGCCCCTCAGAGGGATTCAGAGGGCAGGCTCCAGGCTGACCCACAGAGGTTTCCCCATGGCATCAGGCAGCTGGCCAATT | |
| | ACGTGCACAGCAAAGGCCTGAAGCTGGGGATCTATGCCGATGTGGGGAACAAGACCTGTGCCGGATTTCCAGGCAGCTTTGGCTACTA | |
| | TGACATCGATGCCCAGACCTTTGCCGATTGGGGGGTGGATCTCCTGAAATTTGATGGATGCTATTGTGACAGCCTGGAGAACCTGGCC | |
| | GATGGCTACAAACACATGAGCCTGGCCCTGAACAGGACCGGCAGGAGCATCGTGTACAGCTGTGAGTGGCCTCTGTACATGTGGCCCT | |
| | TCCAGAAGCCAAATTACACAGAGATTAGACAGTATTGCAATCATTGGAGGAACTTTGCCGACATTGACGACAGCTGGAAGAGCATCAA | |
| | GAGCATCCTGGACTGGACCTCATTCAATCAGGAGAGGATTGTGGATGTGGCAGGACCAGGGGGCTGGAATGATCCTGACATGCTGGTG | |
| | ATTGGCAACTTTGGACTGAGCTGGAATCAGCAGGTCACCCAGATGGCACTGTGGGCTATCATGGCCGCCCCCCTGTTTATGAGCAATG | |
| | ACCTCAGGCACATCTCACCACAAGCCAAGGCACTGCTCCAGGATAAGGATGTCATTGCAATTAATCAGGATCCACTGGGCAAACAAGG | |
| | CTATCAGCTGAGACAGGGAGATAATTTTGAGGTGTGGGAGAGGCCTCTGTCTGGCCTGGCATGGGCCGTGGCTATGATCAACAGACAG | |
| | GAGATCGGCGGCCCAAGATCATACACAATCGCCGTGGCATCACTGGGCAAGGGAGTGGCCTGCAACCCTGCCTGCTTTATCACTCAGC | |
| | TGCTGCCTGTGAAAAGGAAGCTGGGCTTCTATGAGTGGACAAGCAGACTGAGGAGCCACATCAACCCCACAGGAACAGTCCTGCTCCA | |
| | ACTGGAAAATACTATGCAAATGTCACTGAAAGATCTGCTGTGAgctagctcgagcgaCTGTGCCTTCTAGTTGCCAGCCATCTGTTGT | |
| | TTGCCCCTCCCCCGTGCCTTCCTTGACCCTGGAAGGTGCCACTCCCACTGTCCTTTCCTAATAAAATGAGGAAATTGCATCGCATTGT | |
| | CTGAGTAGGTGTCATTCTATTCTGGGGGGTGGGGTGGGGCAGGACAGCAAGGGGGAGGATTGGGAAGACAATAGCAGGCATGCTGGGG | |
| | 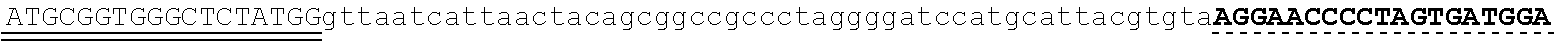 | |
| | 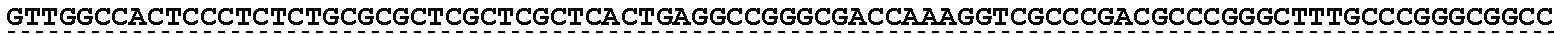 | |
| | 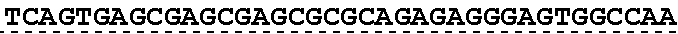 | |
| Construct | 5′ITR (SEQ ID NO: 17), Apo E/C-I (SEQ ID NO: 19), A1AT (SEQ ID NO: 20), β-globin intron | 59 |
| 9B | (SEQ ID NO: 21) , KOZAK consensus leader sequence (SEQ ID NO: 22), IgG1 coC-CpG signal | |
| | --------------------------------------------127----------------------------------------- | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGGGGTGGTCATGTATTATCCTGTTTCTGGTGGCCACAGCCACAGGCGTGCACAGCCTGGACAATG | |
| | GGCTGGCCAGGACTCCAACCATGGGCTGGCTCCACTGGGAGAGGTTCATGTGTAATCTGGACTGCCAAGAAGAACCAGATAGCTGTAT | |
| | CTCTGAGAAACTGTTTATGGAAATGGCTGAGCTCATGGTCAGCGAGGGCTGGAAGGATGCCGGCTACGAGTATCTGTGTATCGATGAC | |
| | TGCTGGATGGCCCCTCAGAGGGATTCAGAGGGCAGGCTCCAGGCTGACCCACAGAGGTTTCCCCATGGCATCAGGCAGCTGGCCAATT | |
| | ACGTGCACAGCAAAGGCCTGAAGCTGGGGATCTATGCCGATGTGGGGAACAAGACCTGTGCCGGATTTCCAGGCAGCTTTGGCTACTA | |
| | TGACATCGATGCCCAGACCTTTGCCGATTGGGGGGTGGATCTCCTGAAATTTGATGGATGCTATTGTGACAGCCTGGAGAACCTGGCC | |
| | GATGGCTACAAACACATGAGCCTGGCCCTGAACAGGACCGGCAGGAGCATCGTGTACAGCTGTGAGTGGCCTCTGTACATGTGGCCCT | |
| | TCCAGAAGCCAAATTACACAGAGATTAGACAGTATTGCAATCATTGGAGGAACTTTGCCGACATTGACGACAGCTGGAAGAGCATCAA | |
| | GAGCATCCTGGACTGGACCTCATTCAATCAGGAGAGGATTGTGGATGTGGCAGGACCAGGGGGCTGGAATGATCCTGACATGCTGGTG | |
| | ATTGGCAACTTTGGACTGAGCTGGAATCAGCAGGTCACCCAGATGGCACTGTGGGCTATCATGGCCGCCCCCCTGTTTATGAGCAATG | |
| | ACCTCAGGCACATCTCACCACAAGCCAAGGCACTGCTCCAGGATAAGGATGTCATTGCAATTAATCAGGATCCACTGGGCAAACAAGG | |
| | CTATCAGCTGAGACAGGGAGATAATTTTGAGGTGTGGGAGAGGCCTCTGTCTGGCCTGGCATGGGCCGTGGCTATGATCAACAGACAG | |
| | GAGATCGGCGGCCCAAGATCATACACAATCGCCGTGGCATCACTGGGCAAGGGAGTGGCCTGCAACCCTGCCTGCTTTATCACTCAGC | |
| | TGCTGCCTGTGAAAAGGAAGCTGGGCTTCTATGAGTGGACAAGCAGACTGAGGAGCCACATCAACCCCACAGGAACAGTCCTGCTCCA | |
| | 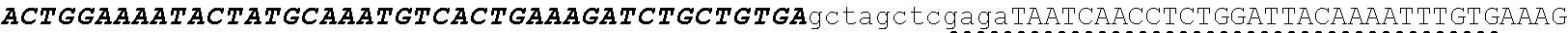 | |
| | 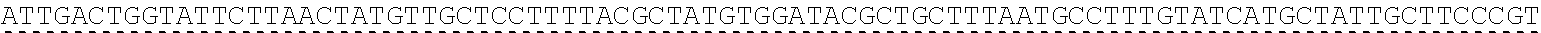 | |
| | 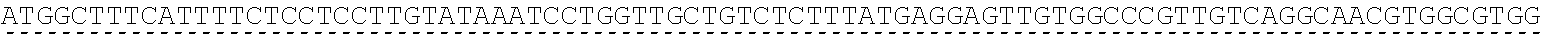 | |
| | 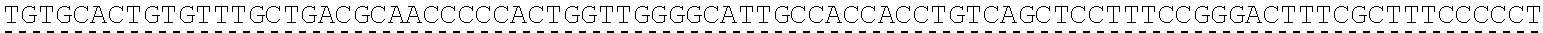 | |
| | 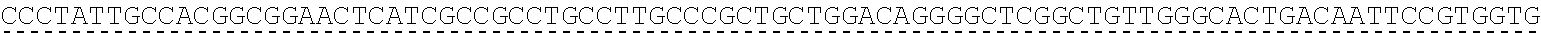 | |
| | 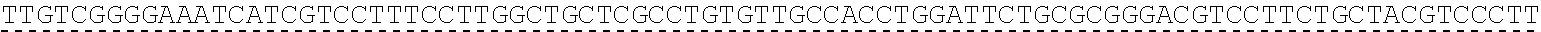 | |
| | 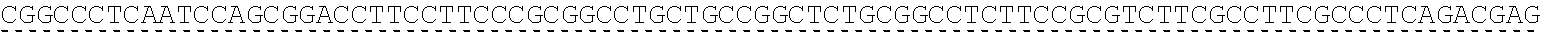 | |
| | 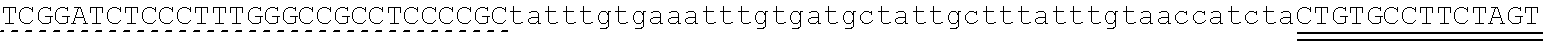 | |
| | TGCCAGCCATCTGTTGTTTGCCCCTCCCCCGTGCCTTCCTTGACCCTGGAAGGTGCCACTCCCACTGTCCTTTCCTAATAAAATGAGG | |
| | AAATTGCATCGCATTGTCTGAGTAGGTGTCATTCTATTCTGGGGGGTGGGGTGGGGCAGGACAGCAAGGGGGAGGATTGGGAAGACAA | |
| | 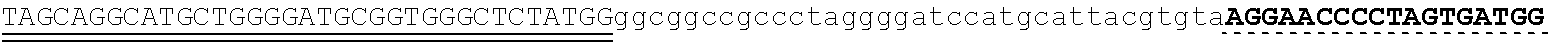 | |
| | 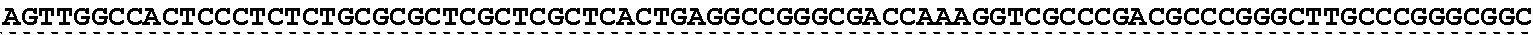 | |
| | 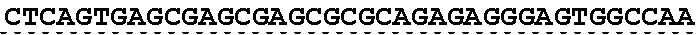 | |
| Construct | 5′ITR (SEQ ID NO: 17), Apo E/C-I (SEQ ID NO: 19), A1AT (SEQ ID NO: 20), β-globin intron | 60 |
| 10B | (SEQ ID NO: 21) , KOZAK consensus leader sequence (SEQ ID NO: 22), IgG1 coC-CpG signal | |
| | 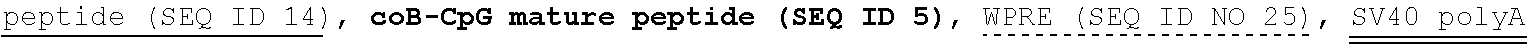 | |
| | 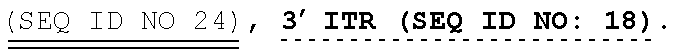 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGGGGTGGTCATGTATTATCCTGTTTCTGGTGGCCACAGCCACAGGCGTGCACAGCCTGGACAATG | |
| | GGCTGGCCAGGACTCCAACCATGGGCTGGCTCCACTGGGAGAGGTTCATGTGTAATCTGGACTGCCAAGAAGAACCAGATAGCTGTAT | |
| | CTCTGAGAAACTGTTTATGGAAATGGCTGAGCTCATGGTCAGCGAGGGCTGGAAGGATGCCGGCTACGAGTATCTGTGTATCGATGAC | |
| | TGCTGGATGGCCCCTCAGAGGGATTCAGAGGGCAGGCTCCAGGCTGACCCACAGAGGTTTCCCCATGGCATCAGGCAGCTGGCCAATT | |
| | ACGTGCACAGCAAAGGCCTGAAGCTGGGGATCTATGCCGATGTGGGGAACAAGACCTGTGCCGGATTTCCAGGCAGCTTTGGCTACTA | |
| | TGACATCGATGCCCAGACCTTTGCCGATTGGGGGGTGGATCTCCTGAAATTTGATGGATGCTATTGTGACAGCCTGGAGAACCTGGCC | |
| | GATGGCTACAAACACATGAGCCTGGCCCTGAACAGGACCGGCAGGAGCATCGTGTACAGCTGTGAGTGGCCTCTGTACATGTGGCCCT | |
| | TCCAGAAGCCAAATTACACAGAGATTAGACAGTATTGCAATCATTGGAGGAACTTTGCCGACATTGACGACAGCTGGAAGAGCATCAA | |
| | GAGCATCCTGGACTGGACCTCATTCAATCAGGAGAGGATTGTGGATGTGGCAGGACCAGGGGGCTGGAATGATCCTGACATGCTGGTG | |
| | ATTGGCAACTTTGGACTGAGCTGGAATCAGCAGGTCACCCAGATGGCACTGTGGGCTATCATGGCCGCCCCCCTGTTTATGAGCAATG | |
| | ACCTCAGGCACATCTCACCACAAGCCAAGGCACTGCTCCAGGATAAGGATGTCATTGCAATTAATCAGGATCCACTGGGCAAACAAGG | |
| | CTATCAGCTGAGACAGGGAGATAATTTTGAGGTGTGGGAGAGGCCTCTGTCTGGCCTGGCATGGGCCGTGGCTATGATCAACAGACAG | |
| | GAGATCGGCGGCCCAAGATCATACACAATCGCCGTGGCATCACTGGGCAAGGGAGTGGCCTGCAACCCTGCCTGCTTTATCACTCAGC | |
| | TGCTGCCTGTGAAAAGGAAGCTGGGCTTCTATGAGTGGACAAGCAGACTGAGGAGCCACATCAACCCCACAGGAACAGTCCTGCTCCA | |
| | 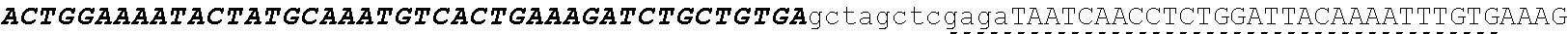 | |
| | 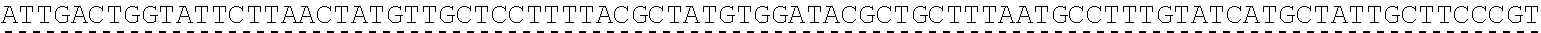 | |
| | 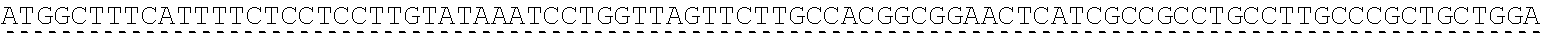 | |
| | 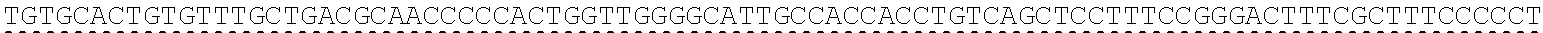 | |
| | 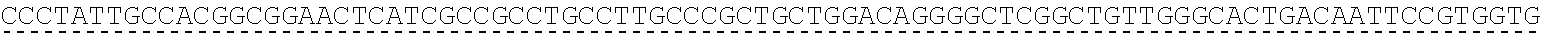 | |
| | 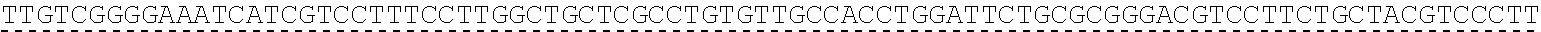 | |
| | 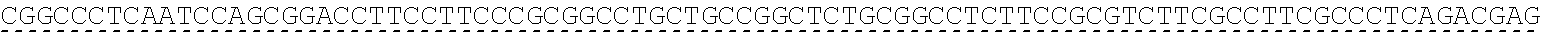 | |
| | 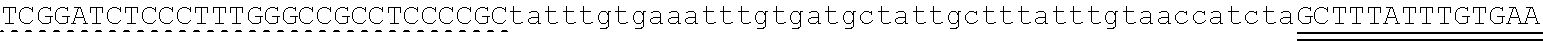 | |
| | ATTGACTGGTATTCTTAACTATGTTGCTCCTTTTACGCTATGTGGATACGCTGCTTTAATGCCTTTGTATCATGCTATTGCTTCCCGT | |
| | ATGGCTTTCATTTTCTCCTCCTTGTATAAATCCTGGTTGCTGTCTCTTTATGAGGAGTTGTGGCCCGTTGTCAGGCAACGTGGCGTGG | |
| | TGTGCACTGTGTTTGCTGACGCAACCCCCACTGGTTGGGGCATTGCCACCACCTGTCAGCTCCTTTCCGGGACTTTCGCTTTCCCCCT | |
| | CCCTATTGCCACGGCGGAACTCATCGCCGCCTGCCTTGCCCGCTGCTGGACAGGGGCTCGGCTGTTGGGCACTGACAATTCCGTGGTG | |
| | TTGTCGGGGAAATCATCGTCCTTTCCTTGGCTGCTCGCCTGTGTTGCCACCTGGATTCTGCGCGGGACGTCCTTCTGCTACGTCCCTT | |
| | CGGCCCTCAATCCAGCGGACCTTCCTTCCCGCGGCCTGCTGCCGGCTCTGCGGCCTCTTCCGCGTCTTCGCCTTCGCCCTCAGACGAG | |
| | TCGGATCTCCCTTTGGGCCGCCTCCCCGCtatttgtgaaatttgtgatgctattgctttatttgtaaccatctaGCTTTATTTGTGAA | |
| | ATTTGTGATGCTATTGCTTTATTTGTAACCATTATAAGCTGCAATAAACAAGTTAACAACAACAATTGCATTCATTTTATGTTTCAGG | |
| | 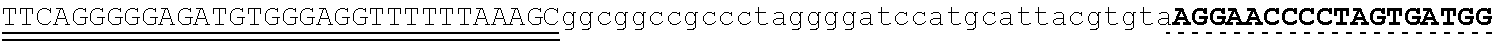 | |
| | 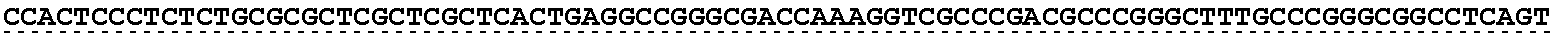 | |
| | 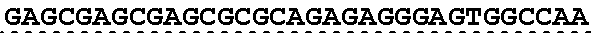 | |
| Construct | 5′ITR (SEQ ID NO: 17), Apo E/C-I (SEQ ID NO: 19), A1AT (SEQ ID NO: 20), β-globin intron | 61 |
| 11B | (SEQ ID NO: 21) , KOZAK consensus leader sequence (SEQ ID NO: 22), IgG1 coC-CpG signal | |
| | 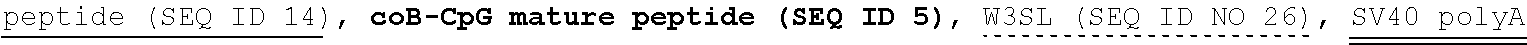 | |
| | 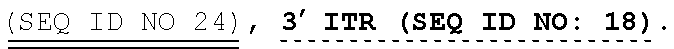 | |
| | TTGGCCACTCCCTCTCTGCGCGCTCGCTCGCTCACTGAGGCCGCCCGGGCAAAGCCCGGGCGTCGGGCGACCTTTGGTCGCCCGGCCT | |
| | CAGTGAGCGAGCGAGCGCGCAGAGAGGGAGTGGCCAACTCCATCACTAGGGGTTCCTtacgtaggacgtcccctgcaggcagtgtagt | |
| | taatgattaacccgccatgctacttatctacgtagccatgctctagaggatcaAGGCTCAGAGGCACACAGGAGTTTCTGGGCTCACC | |
| | CTGCCCCCTTCCAACCCCTCAGTTCCCATCCTCCAGCAGCTGTTTGTGTGCTGCCTCTGAAGTCCACACTGAACAAACTTCAGCCTAC | |
| | TCATGTCCCTAAAATGGGCAAACATTGCAAGCAGCAAACAGCAAACACACAGCCCTCCCTGCCTGCTGACCTTGGAGCTGGGGCAGAG | |
| | GTCAGAGACCTCTCTGGGCCCATGCCACCTCCAACATCCACTCGACCCCTTGGAATTTCGGTGGAGAGGAGCAGAGGTTGTCCTGGCG | |
| | TGGTTTAGGTAGTGTGAGAGGGgtacccgggGATCTTGCTACCAGTGGAACAGCCACTAAGGATTCTGCAGTGAGAGCAGAGGGCCAG | |
| | CTAAGTGGTACTCTCCCAGAGACTGTCTGACTCACGCCACCCCCTCCACCTTGGACACAGGACGCTGTGGTTTCTGAGCCAGGTACAA | |
| | TGACTCCTTTCGGTAAGTGCAGTGGAAGCTGTACACTGCCCAGGCAAAGCGTCCGGGCAGCGTAGGCGGGCGACTCAGATCCCAGCCA | |
| | GTGGACTTAGCCCCTGTTTGCTCCTCCGATAACTGGGGTGACCTTGGTTAATATTCACCAGCAGCCTCCCCCGTTGCCCCTCTGGATC | |
| | CACTGCTTAAATACGGACGAGGACAGGGCCCTGTCTCCTCAGCTTCAGGCACCACCACTGACCTGGGACAGTgaatagatcctgagaa | |
| | cttcaggGTGAGTCTATGGGACCCTTGATGTTTTCTTTCCCCTTCTTTTCTATGGTTAAGTTCATGTCATAGGAAGGGGAGAAGTAAC | |
| | AGGGTACACATATTGACCAAATCAGGGTAATTTTGCATTTGTAATTTTAAAAAATGCTTTCTTCTTTTAATATACTTTTTTGTTTATC | |
| | TTATTTCTAATACTTTCCCTAATCTCTTTCTTTCAGGGCAATAATGATACAATGTATCATGCCTCTTTGCACCATTCTAAAGAATAAC | |
| | AGTGATAATTTCTGGGTTAAGGCAATAGCAATATTTCTGCATATAAATATTTCTGCATATAAATTGTAACTGATGTAAGAGGTTTCAT | |
| | ATTGCTAATAGCAGCTACAATCCAGCTACCATTCTGCTTTTATTTTCTGGTTGGGATAAGGCTGGATTATTCTGAGTCCAAGCTAGGC | |
| | CCTTTTGCTAATCTTGTTCATACCTCTTATCTTCCTCCCACAGctcctgggcaacctgctggtctctctgctggcccatcactttggc | |
| | aaagcacgcgtcGCCGCCACCATGGGGTGGTCATGTATTATCCTGTTTCTGGTGGCCACAGCCACAGGCGTGCACAGCCTGGACAATG | |
| | GGCTGGCCAGGACTCCAACCATGGGCTGGCTCCACTGGGAGAGGTTCATGTGTAATCTGGACTGCCAAGAAGAACCAGATAGCTGTAT | |
| | CTCTGAGAAACTGTTTATGGAAATGGCTGAGCTCATGGTCAGCGAGGGCTGGAAGGATGCCGGCTACGAGTATCTGTGTATCGATGAC | |
| | TGCTGGATGGCCCCTCAGAGGGATTCAGAGGGCAGGCTCCAGGCTGACCCACAGAGGTTTCCCCATGGCATCAGGCAGCTGGCCAATT | |
| | ACGTGCACAGCAAAGGCCTGAAGCTGGGGATCTATGCCGATGTGGGGAACAAGACCTGTGCCGGATTTCCAGGCAGCTTTGGCTACTA | |
| | TGACATCGATGCCCAGACCTTTGCCGATTGGGGGGTGGATCTCCTGAAATTTGATGGATGCTATTGTGACAGCCTGGAGAACCTGGCC | |
| | GATGGCTACAAACACATGAGCCTGGCCCTGAACAGGACCGGCAGGAGCATCGTGTACAGCTGTGAGTGGCCTCTGTACATGTGGCCCT | |
| | TCCAGAAGCCAAATTACACAGAGATTAGACAGTATTGCAATCATTGGAGGAACTTTGCCGACATTGACGACAGCTGGAAGAGCATCAA | |
| | GAGCATCCTGGACTGGACCTCATTCAATCAGGAGAGGATTGTGGATGTGGCAGGACCAGGGGGCTGGAATGATCCTGACATGCTGGTG | |
| | ATTGGCAACTTTGGACTGAGCTGGAATCAGCAGGTCACCCAGATGGCACTGTGGGCTATCATGGCCGCCCCCCTGTTTATGAGCAATG | |
| | ACCTCAGGCACATCTCACCACAAGCCAAGGCACTGCTCCAGGATAAGGATGTCATTGCAATTAATCAGGATCCACTGGGCAAACAAGG | |
| | CTATCAGCTGAGACAGGGAGATAATTTTGAGGTGTGGGAGAGGCCTCTGTCTGGCCTGGCATGGGCCGTGGCTATGATCAACAGACAG | |
| | GAGATCGGCGGCCCAAGATCATACACAATCGCCGTGGCATCACTGGGCAAGGGAGTGGCCTGCAACCCTGCCTGCTTTATCACTCAGC | |
| | TGCTGCCTGTGAAAAGGAAGCTGGGCTTCTATGAGTGGACAAGCAGACTGAGGAGCCACATCAACCCCACAGGAACAGTCCTGCTCCA | |
| | 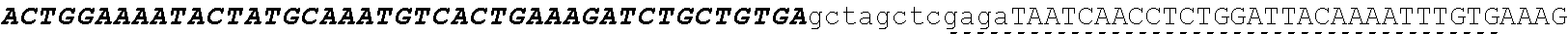 | |
| | 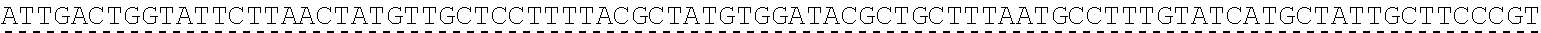 | |
| | 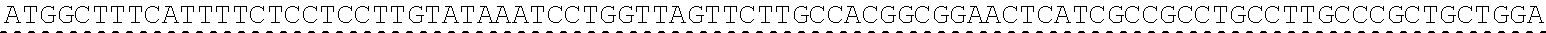 | |
| | 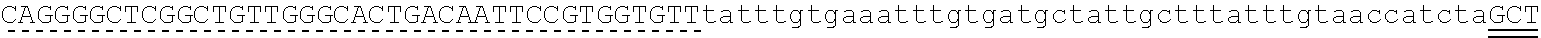 | |
| | TTATTTGTGAAATTTGTGATGCTATTGCTTTATTTGTAACCATTATAAGCTGCAATAAACAAGTTAACAACAACAATTGCATTCATTT | |
| | 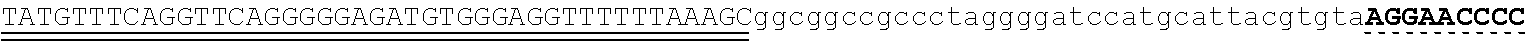 | |
| | 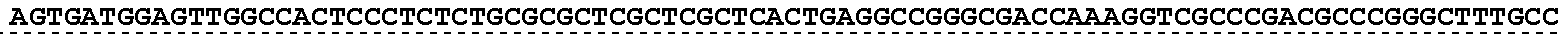 | |
| | 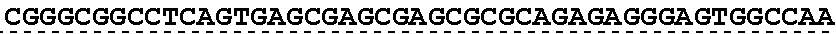 | |

What is claimed is:

1. An isolated recombinant adeno-associated virus (rAAV) particle comprising an AAV capsid protein, wherein the capsid protein comprises an amino acid sequence of SEQ ID NO: 45, or an amino acid sequence at least 85% identical thereto, and a nucleic acid comprising a transgene encoding an alpha-galactosidase (GAL) protein.

2. The isolated rAAV particle of claim 1, wherein the nucleic acid encoding the capsid protein comprises a nucleotide sequence of SEQ ID NO: 46, or a nucleotide sequence at least 85% identical thereto.

3. The isolated rAAV particle of claim 1 or 2, wherein the encoded GAL protein comprises the amino acid sequence of SEQ ID NO: 1, or an amino acid sequence at least 85% identical thereto.

4. The isolated rAAV particle of any one of claims 1-3, wherein the transgene encoding the GAL protein comprises the nucleotide sequence of SEQ ID NO: 2, or a nucleotide sequence at least 85% identical thereto.

5. The isolated rAAV particle of any one of claims 1-3, wherein the transgene encoding the GAL protein is codon optimized.

6. The isolated rAAV particle of claim 5, wherein the transgene encoding the GAL protein comprises the nucleotide sequence of any one of SEQ ID NOs: 3-5, or a nucleotide sequence at least 85% identical thereto.

7. The isolated rAAV particle of any one of claims 1-6, wherein the transgene encoding the GAL protein further encodes a signal sequence.

8. The isolated rAAV particle of claim 7, wherein the encoded signal sequence comprises the amino acid sequence of SEQ ID NO: 6, or an amino acid sequence at least 70% identical thereto.

9. The isolated rAAV particle of claim 7 or 8, wherein the encoded signal sequence is encoded by a nucleic acid comprising the nucleotide sequence of SEQ ID NO: 7, or a nucleotide sequence at least 70% identical thereto.

10. The isolated rAAV particle of claims 7 or 8, wherein the encoded signal sequence is encoded by a codon optimized nucleic acid.

11. The isolated rAAV particle of claim 10, wherein the codon optimized nucleic acid comprises the nucleotide sequence of any one of SEQ ID NOs: 8-10, or a nucleotide sequence at least 70% identical thereto.

12. The isolated rAAV particle of claim 7, wherein the encoded signal sequence comprises the amino acid sequence of SEQ ID NO: 11, or an amino acid sequence at least 70% identical thereto.

13. The isolated rAAV particle of claim 7 or 12, wherein the encoded signal sequence is encoded by a nucleic acid comprising the nucleotide sequence of SEQ ID NO: 12, or a nucleotide sequence at least 70% identical thereto.

14. The isolated rAAV particle of claim 7 or 12, wherein the encoded signal sequence is encoded by a codon optimized nucleic acid.

15. The isolated rAAV particle of claim 14, wherein the codon optimized nucleic acid comprises the nucleotide sequence of any one of SEQ ID NOs: 13-14, or a nucleotide sequence at least 70% identical thereto.

16. The isolated rAAV particle of any one of claims 1-15, wherein the transgene encoding the GAL protein comprises in 5′ to 3′ order:
(i) a nucleotide sequence encoding a signal sequence comprising the nucleotide sequence of any one of SEQ ID NOs: 7-10, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto; or
(ii) a nucleotide sequence encoding a signal sequence comprising the nucleotide sequence of any one of SEQ ID NOs: 12-14, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto.

17. The isolated rAAV particle of any one of claims 1-16, wherein the transgene encoding the GAL protein comprises in 5′ to 3′ order:
(i) a nucleotide sequence encoding a signal sequence comprising the nucleotide sequence of SEQ ID NO: 7, or a nucleotide sequence at least 70% identical thereto; and a nucleotide sequence encoding a GAL protein comprising the nucleotide sequence of SEQ ID NO: 2, or a nucleotide sequence at least 85% identical thereto;
(ii) a nucleotide sequence encoding a signal sequence comprising the nucleotide sequence of SEQ ID NO: 8, or a nucleotide sequence at least 70% identical thereto; and a nucleotide sequence encoding a GAL protein comprising the nucleotide sequence of SEQ ID NO: 3, or a nucleotide sequence at least 85% identical thereto;
(iii) a nucleotide sequence encoding a signal sequence comprising the nucleotide sequence of SEQ ID NO: 9, or a nucleotide sequence at least 70% identical thereto; and a nucleotide sequence encoding a GAL protein comprising the nucleotide sequence of SEQ ID NO: 4, or a nucleotide sequence at least 85% identical thereto;
(iv) a nucleotide sequence encoding a signal sequence comprising the nucleotide sequence of SEQ ID NO: 10, or a nucleotide sequence at least 70% identical thereto; and a nucleotide sequence encoding a GAL protein comprising the nucleotide sequence of SEQ ID NO: 5, or a nucleotide sequence at least 85% identical thereto;
(v) a nucleotide sequence encoding a signal sequence comprising the nucleotide sequence of SEQ ID NO: 12, or a nucleotide sequence at least 70% identical thereto; and a nucleotide sequence encoding a GAL protein comprising the nucleotide sequence of SEQ ID NO: 2;
(vi) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 3, or a nucleotide sequence at least 85% identical thereto;
(vii) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 4, or a nucleotide sequence at least 85% identical thereto; or
(viii) a nucleotide sequence encoding a signal sequence comprising the nucleotide sequence of SEQ ID NO: 14, or a nucleotide sequence at least 70% identical thereto; and a nucleotide sequence encoding a GAL protein comprising the nucleotide sequence of SEQ ID NO: 5, or a nucleotide sequence at least 85% identical thereto;

18. The isolated rAAV particle of any one of claims 1-17, wherein the transgene encoding the GAL protein encodes in 5′ to 3′ order:
(i) a signal sequence comprising the amino acid sequence of SEQ ID NO: 6, or an amino acid sequence at least 70% identical thereto; and a GAL protein comprising the amino acid sequence of SEQ ID NO: 1, or an amino acid sequence at least 85% identical thereto; or
(ii) a signal sequence comprising the amino acid sequence of SEQ ID NO: 11, or an amino acid sequence at least 70% identical thereto; and a GAL protein comprising the amino acid sequence of SEQ ID NO: 1, or an amino acid sequence at least 85% identical thereto.

19. The isolated rAAV particle of any one of claims 1-18, further comprising a promoter operably linked to the nucleic acid comprising the transgene encoding the GAL protein.

20. The isolated rAAV particle of claim 19, wherein the promoter comprises a tissue specific promoter or a ubiquitous promoter.

21. The isolated rAAV particle of claim 19 or 20, wherein the promoter comprises:
(i) an A1AT promoter, an EF-1a promoter, a chicken β-actin (CBA) promoter and/or its derivative CAG, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter), a liver promoter, a skeletal muscle promoter or a fragment, e.g., a truncation, or a functional variant thereof; and/or
(ii) the nucleotide sequence of SEQ ID NOs: 20, or a nucleotide sequence at least 95% identical thereto.

22. The isolated rAAV particle of any one of claims 1-21, further comprising an inverted terminal repeat (ITR) sequence.

23. The isolated rAAV particle of claim 22, wherein the ITR sequence is positioned 5′ relative to the nucleic acid comprising the transgene encoding the GAL protein.

24. The isolated rAAV particle of claim 22, wherein the ITR sequence is positioned 3′ relative to the nucleic acid comprising the transgene encoding the GAL protein.

25. The isolated rAAV particle of claim 22, which comprises an ITR sequence positioned 5′ relative to the nucleic acid comprising the transgene encoding the GAL protein and an ITR sequence positioned 3′ relative to the nucleic acid comprising the transgene encoding the GAL protein.

26. The isolated rAAV particle of any one of claims 22-25, wherein the ITR sequence comprises a nucleotide sequence of SEQ ID NO: 17 and/or 18, or a nucleotide sequence at least 95% identical thereto.

27. The isolated rAAV particle of any one of claims 1-26, further comprising an enhancer.

28. The isolated rAAV particle of claim 27, wherein the enhancer comprises the nucleotide sequence of SEQ ID NO: 19, or a nucleotide sequence at least 95% identical thereto.

29. The isolated rAAV particle of any one of claims 1-18, further comprising an intron region.

30. The isolated rAAV particle of claim 29, wherein the intron region comprises the nucleotide sequence of SEQ ID NO:21, or a nucleotide sequence at least 95% identical thereto.

31. The isolated rAAV particle of any one of claims 1-30, further comprising a Kozak sequence.

32. The isolated rAAV particle of claim 31, wherein the Kozak sequence comprises the nucleotide sequence of SEQ ID NO: 22, or a nucleotide sequence at least 95% identical thereto.

33. The isolated rAAV particle of any one of claims 1-32, further comprising a polyadenylation (polyA) signal region.

34. The isolated rAAV particle of claim 33, wherein the polyA signal region comprises the nucleotide sequence of SEQ ID NO:23, or a nucleotide sequence at least 95% identical thereto.

35. The isolated rAAV particle of claim 33, wherein the polyA signal region comprises the nucleotide sequence of SEQ ID NO:24, or a nucleotide sequence at least 95% identical thereto.

36. The isolated rAAV particle of any one of claims 1-35, further comprising a Woodchuck Hepatitis Virus Posttranscriptional Regulatory Element (WPRE) sequence.

37. The isolated rAAV particle of claim 36, wherein the WPRE sequence comprises the nucleotide sequence of SEQ ID NO: 25, or a nucleotide sequence at least 95% identical thereto.

38. The isolated rAAV particle of claim 36, wherein the WPRE sequence comprises the nucleotide sequence of SEQ ID NO: 26, or a nucleotide sequence at least 95% identical thereto.

39. The isolated rAAV particle of any one of claims 1-38, which comprises in 5′ to 3′ order, a 5′ ITR sequence, an enhancer, a promoter sequence, a Kozak sequence, a nucleotide sequence encoding a signal sequence, a nucleotide sequence encoding a GAL protein, a polyA signal region, and a 3′ ITR sequence.

40. The isolated rAAV particle of any one of claims 1-39, which comprises in 5′ to 3′ order, a 5′ ITR sequence, an enhancer, a promoter sequence, a Kozak sequence, a nucleotide sequence encoding a signal sequence, a nucleotide sequence encoding a GAL protein, a WPRE sequence, a polyA signal region, and a 3′ ITR sequence.

41. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 7, or a nucleotide sequence at least 85% identical thereto;
(vii) a nucleotide sequence encoding a GAL protein comprising the nucleotide sequence of SEQ ID NO: 2 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto;

42. The isolated rAAV particle of any one of claims 1-40, which comprises in 5′ to 3′ order
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 8, or a nucleotide sequence at least 85% identical thereto;
(vii) a nucleotide sequence encoding a GAL protein comprising the nucleotide sequence of SEQ ID NO: 3 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

43. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 9, or a nucleotide sequence at least 85% identical thereto;
(vii) a nucleotide sequence encoding a GAL protein comprising the nucleotide sequence of SEQ ID NO: 4 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

44. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 10, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

45. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 12, or a nucleotide sequence at least 85% identical thereto;
(vii) a nucleotide sequence encoding a GAL protein comprising the nucleotide sequence of SEQ ID NO: 2 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

46. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 13, or a nucleotide sequence at least 85% identical thereto;
(vii) a nucleotide sequence encoding a GAL protein comprising the nucleotide sequence of SEQ ID NO: 3 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

47. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 13, or a nucleotide sequence at least 85% identical thereto;
(vii) a nucleotide sequence encoding a GAL protein comprising the nucleotide sequence of SEQ ID NO: 4 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

48. The isolated rAAV particle of any one of claims 1-40, which comprises in 5′ to 3′ order,
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a polyadenylation sequence comprising the nucleotide sequence of SEQ ID NO: 23, or a nucleotide sequence at least 95% identical thereto; and
(ix) a 3′ ITR sequence region comprising the nucleotide sequence of SEQ ID NO: 18, or a nucleotide sequence at least 95% identical thereto.

49. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a WPRE sequence comprising the nucleotide sequence of SEQ ID NO: 25, or a nucleotide sequence at least 95% identical thereto;
(ix) a polyadenylation sequence comprising the nucleotide sequence of SEQ ID NO: 23, or a nucleotide sequence at least 95% identical thereto; and
(x) a 3′ ITR sequence region comprising the nucleotide sequence of SEQ ID NO: 18, or a nucleotide sequence at least 95% identical thereto.

50. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a WPRE sequence comprising the nucleotide sequence of SEQ ID NO: 25, or a nucleotide sequence at least 95% identical thereto;
(ix) a polyadenylation sequence comprising the nucleotide sequence of SEQ ID NO: 24, or a nucleotide sequence at least 95% identical thereto; and
(x) a 3′ ITR sequence region comprising the nucleotide sequence of SEQ ID NO: 18, or a nucleotide sequence at least 95% identical thereto.

51. The isolated rAAV particle of any one of claims 1-40, which comprises in 5′ to 3′ order:
(i) a 5′ ITR sequence region comprising the nucleotide sequence of SEQ ID NO: 17, or a nucleotide sequence at least 95% identical thereto;
(ii) an Apo E/C-I enhancer comprising the nucleotide sequence of SEQ ID NO: 19, or a nucleotide sequence at least 95% identical thereto;
(iii) an A1AT promoter comprising the nucleotide sequence of SEQ ID NO: 20, or a nucleotide sequence at least 95% identical thereto;
(iv) an intron comprising the nucleotide sequence of SEQ ID NO: 21, or a nucleotide sequence at least 95% identical thereto;
(v) a Kozak sequence comprising the nucleotide sequence of SEQ ID NO: 22, or a nucleotide sequence at least 95% identical thereto;
(vi) a nucleotide sequence encoding a signal sequence comprising the nucleotide sequence of SEQ ID NO: 14, or a nucleotide sequence at least 85% identical thereto;
(vii) a nucleotide sequence encoding a GAL protein comprising the nucleotide sequence of SEQ ID NO: 5 or a nucleotide sequence at least 85% identical thereto;
(viii) a WPRE sequence comprising the nucleotide sequence of SEQ ID NO: 26, or a nucleotide sequence at least 95% identical thereto;
(ix) a polyadenylation sequence comprising the nucleotide sequence of SEQ ID NO: 24, or a nucleotide sequence at least 95% identical thereto; and
(x) a 3′ ITR sequence region comprising the nucleotide sequence of SEQ ID NO: 18, or a nucleotide sequence at least 95% identical thereto.

52. A composition comprising a first nucleic acid encoding an AAV capsid protein, wherein the capsid protein comprises an amino acid sequence of SEQ ID NO: 45, or an amino acid sequence at least 85% identical thereto, and a second nucleic acid comprising a transgene encoding an alpha-glucosidase (GAL) protein.

53. The composition of claim 52, wherein the first nucleic acid encoding the capsid protein comprises a nucleotide sequence of SEQ ID NO: 46, or a nucleotide sequence at least 85% identical thereto.

54. The composition of claim 52 or 53, wherein the encoded GAL protein comprises the amino acid sequence of SEQ ID NO: 1, or an amino acid sequence at least 85% identical thereto.

55. The composition of any one of claims 52-54, wherein the transgene encoding the GAL protein comprises the nucleotide sequence of SEQ ID NO: 2, or a nucleotide sequence at least 85% identical thereto.

56. The composition of any one of claims 52-54, wherein the transgene encoding the GAL protein is codon optimized.

57. The composition of claim 56, wherein the transgene encoding the GAL protein comprises the nucleotide sequence of any one of SEQ ID NOs: 3-5, or a nucleotide sequence at least 85% identical thereto.

58. The composition of any one of claims 52-57, wherein the transgene encoding the GAL protein further encodes a signal sequence.

59. The composition of claim 58, wherein the encoded signal sequence comprises the amino acid sequence of SEQ ID NO: 6, or an amino acid sequence at least 70% identical thereto.

60. The composition of claim 58 or 59, wherein the encoded signal sequence is encoded by a nucleic acid comprising the nucleotide sequence of SEQ ID NO: 7, or a nucleotide sequence at least 70% identical thereto.

61. The composition of claim 58 or 59, wherein the encoded signal sequence is encoded by a codon optimized nucleic acid.

62. The composition of claim 61, wherein the codon optimized nucleic acid comprises the nucleotide sequence of any one of SEQ ID NOs: 8-10, or a nucleotide sequence at least 70% identical thereto.

63. The composition of claim 58, wherein the encoded signal sequence comprises the amino acid sequence of SEQ ID NO: 11, or an amino acid sequence at least 70% identical thereto.

64. The composition of claim 58 or 63, wherein the encoded signal sequence is encoded by a nucleic acid comprising the nucleotide sequence of SEQ ID NO: 12, or a nucleotide sequence at least 70% identical thereto.

65. The composition of claim 58 or 63, wherein the encoded signal sequence is encoded by a codon optimized nucleic acid.

66. The composition of claim 65, wherein the codon optimized nucleic acid comprises the nucleotide sequence of any one of SEQ ID NOs: 13-14, or a nucleotide sequence at least 70% identical thereto.

67. The composition of any one of claims 52-66, wherein the transgene encoding the GAL protein comprises in 5′ to 3′ order:
(i) a nucleotide sequence encoding a signal sequence comprising the nucleotide sequence of any one of SEQ ID NOs: 7-10, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto; or
(ii) a nucleotide sequence encoding a signal sequence comprising the nucleotide sequence of any one of SEQ ID NOs: 12-14, or a nucleotide sequence at least 70% identical thereto; and a nucleotide sequence encoding a GAL protein comprising the nucleotide sequence of any one of SEQ ID NOs: 2-5, or a nucleotide sequence at least 85% identical thereto.

68. The composition of any one of claims 52-67, wherein the transgene encoding the GAL protein comprises in 5′ to 3′ order:
(i) a nucleotide sequence encoding a signal sequence comprising the nucleotide sequence of SEQ ID NO: 7, or a nucleotide sequence at least 70% identical thereto; and a nucleotide sequence encoding a GAL protein comprising the nucleotide sequence of SEQ ID NO: 2, or a nucleotide sequence at least 85% identical thereto;
(ii) a nucleotide sequence encoding a signal sequence comprising the nucleotide sequence of SEQ ID NO: 8, or a nucleotide sequence at least 70% identical thereto; and a nucleotide sequence encoding a GAL protein comprising the nucleotide sequence of SEQ ID NO: 3, or a nucleotide sequence at least 85% identical thereto;
(iii) a nucleotide sequence encoding a signal sequence comprising the nucleotide sequence of SEQ ID NO: 9, or a nucleotide sequence at least 70% identical thereto; and a nucleotide sequence encoding a GAL protein comprising the nucleotide sequence of SEQ ID NO: 4, or a nucleotide sequence at least 85% identical thereto;
(iv) a nucleotide sequence encoding a signal sequence comprising the nucleotide sequence of SEQ ID NO: 10, or a nucleotide sequence at least 70% identical thereto; and a nucleotide sequence encoding a GAL protein comprising the nucleotide sequence of SEQ ID NO: 5, or a nucleotide sequence at least 85% identical thereto;
(v) a nucleotide sequence encoding a signal sequence comprising the nucleotide sequence of SEQ ID NO: 12, or a nucleotide sequence at least 70% identical thereto; and a nucleotide sequence encoding a GAL protein comprising the nucleotide sequence of SEQ ID NO: 2;
(vi) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 3, or a nucleotide sequence at least 85% identical thereto;
(vii) a nucleotide sequence encoding a signal sequence comprising the nucleotide sequence of SEQ ID NO: 13, or a nucleotide sequence at least 70% identical thereto; and a nucleotide sequence encoding a GAL protein comprising the nucleotide sequence of SEQ ID NO: 4, or a nucleotide sequence at least 85% identical thereto; or
(vi) a nucleotide sequence encoding a signal sequence comprising the nucleotide sequence of SEQ ID NO: 14, or a nucleotide sequence at least 70% identical thereto; and a nucleotide sequence encoding a GAL protein comprising the nucleotide sequence of SEQ ID NO: 5, or a nucleotide sequence at least 85% identical thereto;

69. The composition of any one of claims 52-68, wherein the transgene encoding the GAL protein encodes in 5′ to 3′ order:
(i) a signal sequence comprising the amino acid sequence of SEQ ID NO: 6, an amino acid sequence at least 70% identical thereto; and a GAL protein comprising the amino acid sequence of SEQ ID NO: 1, or an amino acid sequence at least 85% identical thereto; or
(ii) a signal sequence comprising the amino acid sequence of SEQ ID NO: 11, an amino acid sequence at least 70% identical thereto; and a GAL protein comprising the amino acid sequence of SEQ ID NO: 1, or an amino acid sequence at least 85% identical thereto.

70. An isolated nucleic acid comprising a transgene encoding an alpha-glucosidase (GAL) protein, wherein the transgene encoding the GAL protein comprises the nucleotide sequence of any one of SEQ ID NOs: 3-5, or a nucleotide sequence at least 85% identical thereto.

71. The nucleic acid of claim 70, wherein the transgene further encodes a signal sequence.

72. The nucleic acid of claim 71, wherein the encoded signal sequence is encoded by a nucleic acid comprising a nucleotide sequence of any one of SEQ ID NOs: 8-10, or a nucleotide sequence at least 70% identical thereto.

73. The nucleic acid of claim 71, wherein the encoded signal sequence is encoded by a nucleic acid comprising a nucleotide sequence of any one of SEQ ID NOs: 12-14, or a nucleotide sequence at least 70% identical thereto.

74. A composition comprising the nucleic acid of any one of claims 70-73.

75. A cell comprising the isolated rAAV particle of any one of claims 1-51, or the composition of any one of claims 52-69 and 74, or the nucleic acid of any one of claims 70-73.

76. The cell of claim 75, wherein the cell is a mammalian cell, an insect cell, or a bacterial cell.

77. A method of making an isolated recombinant adeno-associated virus (rAAV) particle, the method comprising
(i) providing a host cell comprising a nucleic acid comprising a transgene encoding an alpha-glucosidase (GAL) protein; and
(ii) incubating the host cell under conditions suitable to enclose the transgene in an AAV capsid protein, wherein the capsid protein comprises an amino acid sequence of SEQ ID NO: 45, or an amino acid sequence at least 85% identical thereto; thereby making the isolated rAAV particle.

78. A method of making an isolated recombinant adeno-associated virus (rAAV) particle, the method comprising
(i) providing a host cell comprising a first nucleic acid comprising a transgene encoding an alpha-glucosidase (GAL) protein;
(ii) introducing into the host cell a second nucleic acid encoding an AAV capsid protein, wherein the capsid protein comprises an amino acid sequence of SEQ ID NO: 45, or an amino acid sequence at least 85% identical thereto; and
(iii) incubating the host cell under conditions suitable to enclose the transgene in the AAV capsid protein; thereby making the isolated rAAV particle.

79. The method of claim 76 or 77, wherein the host cell comprises a mammalian cell, an insect cell or a bacterial cell.

80. A pharmaceutical composition comprising an rAAV particle of any one of claims 1-51, and a pharmaceutically acceptable excipient.

81. A method of delivering an exogenous GAL protein to a subject, comprising administering an effective amount of the pharmaceutical composition of claim 79, or the isolated rAAV particle of any one of claims 1-51, thereby delivering the exogenous GAL to the subject.

82. The method of claim 81, wherein the subject has, has been diagnosed with having, or is at risk of having a GAL-associated disease.

83. The method of embodiment 81 or 82, wherein the GAL-associated disease is a lysosomal storage disease.

84. A method of treating a subject having or diagnosed with having a GAL-associated disease comprising administering an effective amount of the pharmaceutical composition of claim 80, or the isolated rAAV particle of any one of claims 1-51, thereby treating the GAL-associated disease in the subject.

85. A method of treating a subject having or diagnosed with having a lysosomal storage disease, comprising administering an effective amount of the pharmaceutical composition of claim 80, or the isolated rAAV particle of any one of claims 1-51, thereby treating the lysosomal storage disease in the subject.

86. The method of any one of claims 82-85, wherein the GAL-associated disease or the lysosomal storage disease is Fabry disease.

87. An isolated recombinant adeno-associated virus (rAAV) particle comprising an AAV viral genome comprising or consisting of the nucleic acid sequence of any one of SEQ ID NO: 31-41 and 51-61, and a capsid protein comprising the amino acid sequence of SEQ ID NO: 45.

88. An isolated recombinant viral genome comprising or consisting of the nucleic acid sequence of any one of SEQ ID NO: 31-41 and 51-61.
